(19) United States
(12) Patent Application Publication
(10) Pub. No.: US 2020/0093936 A1
(43) Pub. Date: Mar. 26, 2020

(54) Therapeutic Targeting of Lipid Nanoparticles

(71) Applicant: The Trustees of the University of Pennsylvania, Philadelphia, PA (US)

(72) Inventors: Vladimir Muzykantov, Bryn Athyn, PA (US); Drew Weissman, Philadelphia, PA (US); Hamideh Parhiz, Philadelphia, PA (US); Vladimir V. Shuvaev, Jenkintown, PA (US); Oscar Marcos-Contreras, Philadelphia, PA (US)

(21) Appl. No.: 16/577,281

(22) Filed: Sep. 20, 2019

Related U.S. Application Data
(60) Provisional application No. 62/734,429, filed on Sep. 21, 2018.

(51) Int. Cl. A61K 47/69; A61K 9/51; A61P 25/00; A61K 47/60; A61K 47/68; A61P 11/00; A61K 48/00; A61K 51/12; A61K 9/127; A61K 38/36

(52) U.S. Cl. CPC A61K 47/6925; A61K 9/5123; A61P 25/00; A61K 47/60; A61K 38/366; A61P 11/00; A61K 48/0033; A61K 51/1234; A61K 9/127; A61K 47/6849

(57) ABSTRACT

The present invention relates to compositions comprising a delivery vehicle conjugated to a targeting domain, wherein the delivery vehicle comprises at least one agent, and wherein the targeting domain specifically binds to an endothelial marker. The invention also relates to methods of treating or preventing neurological or pulmonary conditions using the described compositions.

### CROSS REFERENCE TO RELATED APPLICATIONS

This application claims priority to U.S. Provisional Application No. 62/734,429, filed Sep. 21, 2018, which is hereby incorporated by reference herein in its entirety.

### STATEMENT REGARDING FEDERALLY SPONSORED RESEARCH OR DEVELOPMENT

This invention was made with government support under T32 HL007915 awarded by the National Institute of Health. The government has certain rights in the invention.

### BACKGROUND OF THE INVENTION

RNA-based agents are emerging as potential therapeutic options distinct from DNA-based gene therapy approaches. For example, mRNA, which does not integrate into host genome nor require nuclear delivery, offers transient translation of needed sequence in cells (Weissman & Kariko Mol. Ther. 2015, 23, 1416-1417). While RNA-based therapies are still in their infancy, there are currently more than 30 clinical trials registered for mRNA-based cancer therapeutics and vaccines (Pardi, et al. J. Control. Release 2015, 217, 345-351). Like all drugs and especially biotherapeutics, delivery of mRNA is a major challenge for most organs except liver (Shuvaev, et al., J. Control. Release 2015, 219, 576-595). Drug delivery systems (DDS) including lipid nanoparticles (LNPs) are employed to pack RNA and protect cargo en route to the site of action (Kauffman, et al., J. Control. Release 2016, 240, 227-234). However, targeted delivery and effect of RNA in organs and tissues of interest remains a formidable barrier for the biomedical translation and utility of this class of agents.

Thus there is a need in the art for improved compositions and methods for targeted delivery of RNA and other cargo. The present invention satisfies this unmet need.

### SUMMARY OF THE INVENTION

In one aspect, the present invention relates to a composition comprising a delivery vehicle conjugated to a targeting domain, wherein the delivery vehicle comprises at least one agent, and wherein the targeting domain specifically binds to an endothelial marker selected from the group consisting of ICAM-1, PECAM-1, VCAM-1, ACE, APP, PV1, P-selectin, E-selectin, and VE-cadherin. In one embodiment, the delivery vehicle is selected from the group consisting of a liposome, a lipid nanoparticle, and a micelle. In one embodiment, the delivery vehicle is a lipid nanoparticle. In one embodiment, the lipid nanoparticle comprises a PEG-lipid conjugated to the targeting domain.

In one embodiment, the at least one agent is encapsulated in the lipid nanoparticle. In one embodiment, the at least one agent is selected from the group consisting of a therapeutic agent, an imaging agent, diagnostic agent, a contrast agent, a labeling agent, a detection agent, and a disinfectant. In one embodiment, the at least one agent is a therapeutic agent. In one embodiment, the therapeutic agent comprises a nucleic acid molecule. In one embodiment, the nucleic acid molecule encodes a therapeutic peptide selected from the group consisting of a thrombomodulin, endothelial protein C receptor, an anti-thrombotic protein, a plasminogen activator, catalase, superoxide dismutase, and an iron-sequestering protein.

In one embodiment, wherein the targeting domain is selected from the group consisting of a nucleic acid molecule, a peptide, an antibody, and a small molecule. In one embodiment, the targeting domain is an antibody. In one embodiment, wherein the targeting domain specifically binds to platelet-endothelial cell adhesion molecule-1 (PECAM-1). In one embodiment, the targeting domain specifically binds to vascular cell adhesion molecule-1 (VCAM-1). In one embodiment, the targeting domain specifically binds to intercellular adhesion molecule-1 (ICAM-1).

In another aspect, the present invention relates to a method of treating or preventing a neurological condition of a subject, the method comprising administering to the subject the composition of the invention. In one embodiment, the neurological condition is selected from the group consisting of stroke, inflammation, infection, meningitis, traumatic brain injury, multiple sclerosis, concussion, cerebral embolism, hemorrhage, brain tumors, neurodegenerative disorders, depression, post-traumatic stress disorder, anxiety, mood disorders, and addiction disorders.

In another aspect, the present invention relates to a method of treating or preventing a pulmonary condition of a subject, the method comprising administering to the subject the composition of the invention. In one embodiment, the pulmonary condition is selected from the group consisting of acute lung injury, pulmonary ischemia including organ transplantation, pulmonary embolism, pulmonary edema, pulmonary hypertension, fibrosis, infection, inflammation, emphysema, and cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic illustrating the use of affinity ligands such as antibodies to specific cell surface markers for development of lung-targeted lipid-based nanoparticles. In this embodiment, amino groups on antibodies were functionalized with heterobifunctional crosslinker (SATA) for introduction of thiol moieties on antibody surface followed by maleimide-thiol conjugation to maleimide-bearing LNPs. Other methods for conjugation are available including click-chemistry based techniques.

FIG. 2A through FIG. 2D depict the results of example experiments depicting the physiochemical characterization of nanoparticles. FIG. 2A: The averaged (n=3) intensity size distribution curves for the unmodified LNP (gray trace) and antibody-conjugated LNPs (black and red traces). FIG. 2B: Particle size (z-average) and surface charge of particles measured using dynamic light scattering (DLS) and laser doppler velocimetry (LDV), respectively (n=3). Images taken by transmission electron microscopy of unmodified LNP (FIG. 2C), and antibody-modified LNP (FIG. 2D), scale bar:100 nm.

FIG. 3A through FIG. 3D depict the results of example experiments demonstrating the binding and functional activity of targeted particles in healthy and diseased cellular models. FIG. 3A: In vitro binding of targeted LNPs to PECAM positive and negative REN cells after 1 hour incubation of ¹²⁵I-labeled LNP-anti PECAM with cells at room temperature. (*P<0.05), transfection activity of LNP-anti PECAM in REN-PECAM positive compared to REN-WT. FIG. 3B: In vitro luciferase activity of antibody conjugated Luc-mRNA-LNPs in PECAM positive REN cells. (# P<0.05), transfection activity of LNP-anti PECAM compared to LNP-control IgG. FIG. 3C: In vitro GFP expression of LNP-control IgG and anti-hPECAM conjugated GFP-mRNA-LNPs in HUVEC, 6 μg mRNA per well. FIG. 3D: In vitro luciferase activity of antibody conjugated Luc-mRNA-LNPs in untreated and TNF-α-treated HUVEC. (†P<0.05), transfection activity of LNP-anti ICAM in TNF-α-treated cells compared to untreated cells.

FIG. 4A and FIG. 4B depict the results of example experiments demonstrating the targeting of mRNA loaded nanoparticles to PECAM-1 in vivo. FIG. 4A: Biodistribution of ¹²⁵I-labeled anti-PECAM mAb- and control IgG-LNPs in mice at 30 min. Tissue uptake is indicated as mean±SEM (n=3). (*P<0.05 and **P<0.001), tissue uptake of LNP-anti PECAM compared to LNP-control IgG. FIG. 4B: Immunospecificity index, calculated as the ratio of % ID/g of selected organs in mice treated with targeted (anti-PECAM) vs. non-targeted (control IgG)-LNPs, normalized to blood levels.

FIG. 5A and FIG. 5B depict the results of example experiments demonstrating the in vivo kinetics of LNP binding. FIG. 5A: Quantitative measurement of the percentage of PECAM-targeted mRNA-loaded and unmodified mRNA-loaded (inset) LNPs evaluated by radioactivity analysis in selected organs, after intravenous injection of nanoparticles. FIG. 5B: Localization ratio (the ratio of % ID/g of a given organ to that in the blood) of selected organs after intravenous injection of anti PECAM-targeted mRNA-loaded and unmodified mRNA-loaded (inset) LNPs.

FIG. 6A through FIG. 6C depict the results of example experiments demonstrating the functional activity of targeted luciferase mRNA-loaded nanoparticles to PECAM-1 in vivo. Organ distribution of luciferase mRNA expression 4.5 hours after intravenous administration of unmodified, anti-PECAM mAb-, and control IgG-LNPs demonstrated as luminescence imaging (FIG. 6A) and luciferase activity (FIG. 6B). FIG. 6A shows a representative sample set of mouse organs which are analyzed 5 min after the injection of D-luciferin. FIG. 6B shows quantitative expression as LU/mg protein values compared between non-targeted and targeted LNP. Lung transfection efficiency upon IV administration of anti-PECAM-LNPs increases up to 25 fold compared to Control-IgG-LNP. Data presented as mean±SEM (n=3), (*P<0.05), transfection activity of LNP-anti-PECAM compared to LNP-control IgG. Transfection-specificity index (inset), calculated as the ratio of luciferase activity in selected organs of mice treated with targeted (anti-PECAM) vs. non-targeted (control IgG)-LNPs. FIG. 6C: Lung to liver ratio, calculated as the ratio of transfection efficiency of lung to that of liver for each formulation.

FIG. 7A through FIG. 7B, depict the results of example experiments demonstrating the in vivo kinetics of luciferase expression following LNP administration. FIG. 7A: Quantitative measurement of luciferase activity in liver, kidney, and lung upon intravenous injection of non-targeted and anti PECAM-targeted luciferase mRNA-loaded LNPs; mRNA dose: 8 μg/mouse. FIG. 7B: Dose-response relationship of Luciferase mRNA containing anti-PECAM LNPs. The mice received LNPs at doses of 1, 2, 4, and 8 μg mRNA per mouse via IV administration. Selected organs were then harvested at 4.5 hours post-treatment and luciferase activity was measured in tissue extracts.

FIG. 8 depicts the results of example experiments demonstrating luciferase mRNA expression in ApoE knockout mice. Unmodified, control IgG, and anti-PECAM Luc-mRNA-LNPs were intravenously injected into mice. Mice were sacrificed 4.5 hours after injection and luciferase activity in selected organs of wild-type mice was compared to ApoE knockout mice. Data presented as mean±SEM (n=3); (*P<0.05), transfection activity of LNP-anti-PECAM was compared in wild-type vs. ApoE knockout mice.

FIG. 9A through FIG. 9C depict the results of example experiments. FIG. 9A: Illustration of stereotaxic injection of tumor necrosis factor (TNF). 2.5 μL TNF (200 μg/mL) was administered by Intrastriatal (i.s.) injection using a 10-μl Nanofil microsyringe over a 3-min period. FIG. 9B: Tissue uptake of ¹²⁵I-labeled anti VCAM- and control IgG-LNPs in the ipsilateral hemisphere of brain in healthy (Anti VCAM-LNP and Control IgG-LNP) and TNF-induced brain injured mice (Anti VCAM-LNP-TNF-treated and Control IgG-LNP-TNF-treated) at 30 min. Tissue uptake is indicated as mean±SEM (n=3); tissue uptake obtained from LNP-anti-VCAM was compared to IgG counterpart in both naive and TNF-treated mice (*P<0.05). LNP-anti-VCAM was also compared in TNF-treated mice vs. naive mice (†P<0.05). FIG. 9C: Luciferase mRNA expression in the ipsilateral hemisphere at 4.5 h after intravenous administration of anti PECAM-, anti VCAM-, and control IgG-LNPs to TNF-induced brain inflammation mouse model. Transfection activity of LNP-anti-VCAM compared to LNP-control IgG (*P<0.05), LNP-anti-PECAM (†P<0.05), and LNP-anti-ICAM (# P<0.05).

FIG. 10A through FIG. 10D depict the results of example experiments. FIG. 10A depicts the biodistribution in brain and lungs (inset) of antibodies (IgG, anti-ICAM, anti-VCAM) injected 24 hours after intrastriatal injection of TNF (0.5 μg in 2 μl; TNF/Brain) or in healthy animals. FIG. 10B depicts the biodistribution in brain and lungs (inset) of targeted liposomes (anti-ICAM or anti-VCAM) or control liposomes (IgG) injected 24 hours after intrastriatal injection of TNF (0.5 μg in 2 μl; TNF/Brain) or in healthy animals. FIG. 10C depicts the biodistribution in lung of targeted liposomes (anti-ICAM or anti-VCAM) or control liposomes (IgG) injected intravenously (IV) or intra-arterially (IA) 24 hours after intrastriatal injection of TNF (0.5 μg in 2 μl). FIG. 10D depicts the biodistribution in lung of targeted liposomes (anti-ICAM or anti-VCAM) or control liposomes (IgG) injected intravenously (IV) or intra-arterially (IA) 24 hours after intrastriatal injection of TNF (0.5 μg in 2 μl). Mean±SEM (n=3).

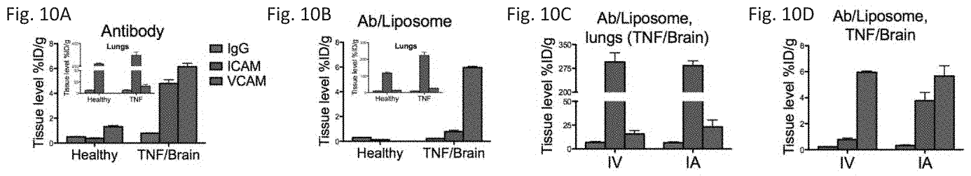

FIG. 11A and FIG. 11B depict the results of example experiments demonstrating the biodistribution of radiolabeled antibodies and immunoliposomes in naive and TNFα injured mice. (FIG. 11A, left panel) Anti-ICAM-1 mAb demonstrates specific uptake in the lung (***—p<0.001 vs. IgG and VCAM-1), with a slight—but statistically significant—increase in animals receiving intrastriatal TNFα (#—p<0.05 vs. naive). Anti-VCAM-1 mAb, in contrast, accumulates in the brain (***—p<0.001 vs. IgG and ICAM-1) and demonstrates a >10-fold increase in both brain uptake (FIG. 11A, middle panel) and brain:blood ratio (FIG. 11A, right panel) following intrastriatal TNFα (***—p<0.001 vs. naive). (FIG. 11B) ICAM-1 and VCAM-1 targeted immunoliposomes show nearly identical patterns of lung and brain biodistribution as their counterpart mAbs. In particular, anti-VCAM-1 liposomes demonstrate a similar ˜10-fold increase in brain uptake (FIG. 11B, middle panel) and brain:blood ratio (FIG. 11B, right panel) in TNFα injured mice (***—p<0.001 vs. naive). In all experiments, organ biodistribution was measured 30 minutes after intravenous injection of radiolabeled materials. mAb or immunoliposomes were given 16 hours after intrastriatal TNFα injection. Each data point represents N=3 animals, with mean±SD shown and Two-way anova with Dunnett's post-hoc test was applied.

FIG. 12A through FIG. 12C depict the results of example experiments using SPECT imaging of immunoliposomes. Three-dimensional reconstructions (FIG. 12A) and average intensity projections (FIG. 12B) of SPECT (red) and CT (grey) signals for intrastriatal TNFα-injured mice receiving IgG or anti-VCAM-1 functionalized liposomes bearing ¹¹¹In-DTPA. Average intensity projections (FIG. 12B) encompassed SPECT and CT signal in the mouse cranium. (FIG. 12C) Autoradiography images generated by anti-VCAM-1 functionalized liposomes bearing ¹¹¹In-DTPA in TNFα-injured brain sections. Arrows indicate the injected hemisphere and dashed lines indicate the separation between the 2 brain hemispheres.

FIG. 13 depicts the results of example experiments demonstrating intravital imaging of cerebrovascular immunoliposome distribution. Intravital microscopy was performed through a cranial window and used to demonstrate real-time localization of fluorescent VCAM-1 targeted (bottom rows) vs. IgG control (top rows) liposomes (green). Merged images also show circulating leukocytes (red) labeled via intravenous injection of rhodamine dye. Left-hand panels show baseline images and the localization of liposomes given 24-hours prior to TNFα injury. Right hand panels show liposomal accumulation 2-hours after TNFα. While enhanced and prolonged fluorescent signal suggest greater uptake, localizaton remains predominantly at the vessel margin, despite massive influx of circulating leukocytes.

FIG. 14A through FIG. 14D depict the results of flow cytometric analysis of cell types involved in immunoliposome uptake. Flow cytometry was performed on disaggregated brains following injection of anti-VCAM-1 or IgG control immunoliposomes. CD31 and CD45 staining were used to identify 4 distinct cell populations: endothelial (CD45⁻CD31⁺), microglia/macrophages (CD45^{Mid}), leukocytes (CD45^{Hi}), and double negative (CD45⁻CD31⁻) cells. Representative 2-D plots from naive (FIG. 14A) and TNF-α injured mice (FIG. 14B) show identification of each cell type (left panel) and the percentage of CD31⁺ and CD31⁻ cells which stained for IgG control (middle panel) and anti-VCAM-1 (right panel) immunoliposomes. (FIG. 14C) While only a small percentage of ECs stain positive for anti-VCAM liposomes in control mice, more than half of recovered ECs are liposome positive in TNF-α injured mice (*—p<0.001). Likewise, the percentage of positive ECs was significantly greater than all other cell types (**—p<0.001). A similar pattern was seen for the mean fluorescence intensity (MFI). The MFI of liposome positive ECs was significantly higher in TNF-α injected vs. control mice (***—p<0.001) and in ECs vs. other cell types (****—p<0.001). Each bar represents N=3 mice with mean±SD shown.

FIG. 15A and FIG. 15B depict the results of example experiments demonstrating that targeted LNP accumulate in the brain and express the encapsulated mRNA. (FIG. 15A) Tissue uptake of ¹²⁵I-labeled anti-VCAM-1- and control IgG-LNPs in the ipsilateral hemisphere of brain in healthy and TNFα-induced brain injured mice at 30 minutes. Tissue uptake is indicated as mean±SEM (n=3); tissue uptake obtained from anti-VCAM-1-LNP was compared to control IgG counterpart in both naive and TNFα-treated mice (***p<0.001, one way anova, Bonferroni post-hoc). Anti-VCAM-1-LNP was also compared in TNFα-treated mice vs. naive mice (### p<0.001 one way anova, Bonferroni post-hoc)). Firefly luciferase mRNA expression (inset) in the ipsilateral hemisphere at 5 hours after intravenous administration of anti-VCAM-1 and anti-ICAM-1-LNP-mRNAs to TNFα-induced brain inflammation mouse model. Transfection activity of anti-VCAM-1-LNP compared to anti-ICAM-1-LNP (***p<0.001, one way anova, Bonferroni post-hoc)). (FIG. 15B) Western blot showing brain homogenates (10 μg total protein/lane) stained for FLAG, TM and a-actin. Mice were treated with anti-VCAM-1 and anti-ICAM-1 targeted LNPs encoding mRNA for TM-FLAG (LNP-TM) intra-arterially (via internal carotid artery) eight hours post-treatment.

FIG. 16A and FIG. 16B depict the results of example experiments evaluating brain edema: extravascular radiolabeled albumin accumulation in the brain. (FIG. 16A) Assessment of brain edema using albumin leakage assay. Radiolabeled albumin was injected 21 hours after unilateral striatal injection of TNFα (0.5 μg) and allowed to circulate for 4 hours. The ratio between extravasated and bloodstream radiolabeled albumin was determined as (cpm/g brain: cpm/g blood). Treatment with TNFα significantly increased albumin leakage in both hemispheres (*, p<0.01 in contralateral and ***, p<0.001 in ipsilateral, compared to PBS treated animals, one way anova, Bonferroni post-hoc)). Data shown as mean±SEM. (FIG. 16B) Treatment with anti-VCAM-1targeted LNP-TM significantly reduced albumin leakage in ipsilateral hemisphere compared to non-targeted LNP-TM and PBS treated animals (***, p<0.001). Data shown as mean±SEM.

FIG. 17A through FIG. 17D depict the results of example experiments demonstrating the biodistribution of radiolabeled antibodies. Tissue uptake (% ID/g), localization ratio (% ID/g Organ/% ID/g Blood) and Immunospecificity Index (ISI; localization ratio targeted mAb/localization ratio untargeted IgG) for major organs for IgG control (FIG. 17A), anti-VCAM-1 mAb (FIG. 17B), anti-ICAM-1 (FIG. 17C) and anti-TfR-1 (FIG. 17D) for naive and TNFα treated animals. Mean±SD.

FIG. 18A through FIG. 18C depict the results of example experiments demonstrating the biodistribution of radiolabeled immunoliposomes. Tissue uptake (% ID/g), localization ratio (% ID/g Organ/% ID/g Blood) and Immunospecificity Index (localization ratio targeted immunoliposome/localization ratio untargeted IgG) for major organs for IgG control (FIG. 18A), anti-VCAM-1 immunoliposome (FIG. 18B), and anti-ICAM-1 immunoliposome (FIG. 18C) for naive and TNFα treated animals. Mean±SD.

FIG. 19 depicts the results of example experiments demonstrating the assessment of brain edema using albumin leakage assay. Radiolabeled albumin was injected 21 or 44 h post unilateral striatal injection of TNFα (0.5 μg) and allowed to circulate for 4 hours. The ratio between extravasated and bloodstream radiolabeled albumin was determined as (cpm/g brain: cpm/g blood). Treatment with TNFα significantly increased albumin leakage in both hemispheres (*, p<0.05 in contralateral and ***, p<0.001 in ipsilateral, compared to PBS naive; ### p<0.001 compared to TNFα 25 hours). Data shown as mean±SEM.

FIG. 20A through FIG. 20E depict the results of example experiments demonstrating the physicochemical characterization of targeted mRNA containing lipid nanoparticles. (FIG. 20A) Schematic illustration of the use of antibodies against endothelial cell surface markers for development of lung-targeted LNPs. Amino groups on antibodies were functionalized with heterobifunctional crosslinker (SATA) for introduction of thiol moieties on antibody surface followed by maleimide-thiol conjugation to maleimide-bearing LNP-mRNAs. (FIG. 20B) The average (n=3) intensity size distribution curves for the unconjugated LNP-mRNA (gray trace) and antibody-conjugated LNP-mRNAs (black and red traces). (FIG. 20C) Particle size (z-average) and surface charge of particles measured using dynamic light scattering (DLS) and laser doppler velocimetry (LDV), respectively (n=3). Images taken by transmission electron microscopy of (FIG. 20D) unconjugated LNP-mRNA, and (FIG. 20E) antibody-conjugated LNP-mRNA, scale bar: 100 nm.

FIG. 21A through FIG. 21C depict the results of example experiments demonstrating the binding and functional activity of targeted particles in vitro. (FIG. 21A) In vitro binding of targeted LNP-mRNAs to PECAM-1 positive and negative REN cells after 1 h incubation of ¹²⁵I-labeled anti-PECAM-1/LNP-mRNA with cells at RT (*P<0.05). (FIG. 21B) mRNA encoded protein expression of anti-PECAM-1/LNP-mRNA in REN-PECAM-1 positive cells compared to control IgG/LNP-mRNA (# P<0.05). The inset shows the luciferase activity for unconjugated LNP-mRNA. (FIG. 21C) In vitro eGFP expression of control IgG and anti-PECAM-1 conjugated eGFP-mRNA-LNPs in REN-PECAM-1 positive cells, 6 μg mRNA per well.

FIG. 22A through FIG. 22E depict the results of example experiments demonstrating that targeting of LNP-mRNA to PECAM-1 in vivo. (FIG. 22A) Biodistribution of ¹²⁵I-labeled anti-PECAM mAb- and control IgG-LNP-mRNAs in mice at 30 min. Tissue uptake is indicated as mean±SEM (n=3). (*P<0.05 and **P<0.001). (FIG. 22B) Immunospecificity index, calculated as the ratio of % ID/g of selected organs in mice treated with targeted (anti-PECAM-1) vs. non-targeted (control IgG)-LNP-mRNAs, normalized to blood levels. In vivo kinetics of LNP-binding as quantitative measurement of the percentage of PECAM-1-targeted (FIG. 22C), Control IgG- (FIG. 22D) and unconjugated (FIG. 22E) mRNA-loaded LNPs evaluated by radioactivity analysis in selected organs, after intravenous injection of nanoparticles.

FIG. 23A and FIG. 23B depict the results of example experiments demonstrating flow cytometric analysis of cell populations receiving PECAM-1 targeted LNPs in lung tissue. Staining was performed against CD31 for endothelial cells, CD45 for leukocytes, and F4/80 for monocytes/macrophages. (FIG. 23A) Pie chart representative of total cell recovery from lung. (FIG. 23B) Percent of sub-cell populations positive for LNPs.

FIG. 24A through FIG. 24D depict the results of example experiments depicting the cell toxicity/inflammatory profile of LNP-mRNA. (FIG. 24A) Effect of anti-PECAM mAb-, control IgG-, and unconjugated LNP-mRNAs on cell viability measured by colorimetric MTS assay. % Viability is indicated as mean±SEM (n=3). (FIG. 24B) Western blot showing cell lysates (10 μg total protein/lane) stained for human VCAM-1 and actin. An increase in VCAM-1 protein expression was induced by LPS, but not by LNP-mRNA treatment. Pro-inflammatory cytokines IL-6 in plasma (FIG. 24C) and MIP-2 in liver homogenate (FIG. 24D) upon treatment with LNP-mRNA (8 μg/mouse) were compared to the untreated samples. LPS (2 mg/kg) was used as positive control here.

FIG. 25A through FIG. 25E depict the results of example experiments. Organ distribution of firefly luciferase mRNA expression 4.5 h after intravenous administration of unconjugated, anti-PECAM-1 mAb- and control IgG/LNP-mRNAs demonstrated as (FIG. 25A) firefly luciferase activity and (FIG. 25B) luminescence imaging. (FIG. 25A) Quantitative expression as LU/mg protein values compared between non-targeted and targeted LNP. Data presented as mean±SEM (n=3), (*P<0.05). (FIG. 25B) A representative sample set of mouse organs, which were analyzed 5 min after the administration of D-luciferin. (FIG. 25C) Transfection-specificity index, calculated as the ratio of luciferase activity in selected organs of mice treated with targeted (anti-PECAM-1) vs. non-targeted (control IgG)-LNP-mRNAs. (FIG. 25D) Lung to liver ratio, calculated as the ratio of transfection efficiency of lung to that of liver for each formulation. (FIG. 25E) Dose-response relationship of Luc mRNA containing anti-PECAM-1-LNPs. Mice received LNPs at doses of 1, 2, 4, and 8 μg mRNA per mouse via intravenous administration. Selected organs were harvested at 4.5 h post-treatment and firefly luciferase activity was measured in tissue extracts.

FIG. 26A and FIG. 26B depict the results of example experiments demonstrating the in vivo kinetics of firefly luciferase expression following LNP-mRNA administration. Quantitative measurement of firefly luciferase activity in (FIG. 26A) liver and (FIG. 26B) lung upon intravenous injection of unconjugated- and anti-PECAM-1/LNP-mRNA; mRNA dose: 8 μg/mouse.

FIG. 27 depicts the results of example experiments demonstrating firefly luciferase mRNA expression in apoE knockout mice. Unconjugated, control IgG, and anti PECAM-1 Luc mRNA-LNPs were intravenously injected into mice. Mice were sacrificed 4.5 h after injection and firefly luciferase activity in livers and lungs of wild type mice was compared to apoE knockout mice. Data presented as mean±SEM (n=3); (*P<0.05).

FIG. 28 depicts the results of example experiments demonstrating antibody modified LNP-mRNA diameter size change upon incubation in varying ionic strength solutions.

FIG. 29 depicts the results of example experiments demonstrating HUVEC transfection with targeted LNP-mRNA. In vitro eGFP expression of control IgG and anti-PECAM-1 conjugated eGFP-mRNA-LNPs in HUVECs, 6 μg mRNA per well.

FIG. 30 depicts the results of experiments demonstrating the quantitative measurement of firefly luciferase activity (LU/mg protein) in selected organs upon intravenous injection of Luc mRNA-LNPs in apoE knockout mice; mRNA dose: 8 μg/mouse. Data presented as mean±SEM (n=3).

### DETAILED DESCRIPTION

The present invention relates to compositions having a delivery vehicle conjugated to a targeting domain, wherein the delivery agent comprises at least one agent. In one embodiment, the targeting domain specifically binds to an endothelial marker. For example, in one embodiment, the targeting domain directs the vehicle to the vasculature or to a specific region of the vasculature. In certain embodiments, the targeting domains directs the vehicle to the cerebral vasculature or pulmonary vasculature.

In certain embodiments, the delivery vehicle is a lipid nanoparticle comprising a PEG-lipid conjugated to the targeting domain. In some embodiments, the at least one agent is a nucleic acid. The present invention also relates to methods of treating pulmonary or neurological conditions related to the vasculature using the compositions described herein.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule, which specifically binds with an antigen or epitope. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. k and l light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl psuedouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, in another embodiment, to m¹acp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an affinity ligand, in particular, an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “therapeutic” as used herein means a treatment and/or prophylaxis. A therapeutic effect is obtained by suppression, diminution, remission, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C₁-C₂₄ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, prop 1 enyl, but-1-enyl, pent-1-enyl, penta-1,4-dienyl, ethynyl, propynyl, butynyl, pentynyl, hexynyl, and the like. Unless specifically stated otherwise, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates in part to compositions and methods for targeted delivery of a delivery vehicle. In one aspect, the present invention relates to composition comprising a delivery vehicle conjugated to a targeting domain. In one embodiment, the delivery vehicle comprises at least one agent, such as a therapeutic agent. In one embodiment, the delivery vehicle comprises RNA, including but not limited to mRNA, nucleoside-modified RNA, siRNA, miRNA, shRNA, or antisense RNA.

In various embodiments, the targeting domain binds to a cell surface molecule of a cell related to the vasculature, such as an endothelial cell. For example, in various embodiments, the targeting domain binds to a molecule selected from the group including, but not limited to, (ICAM-1), platelet-endothelial cell adhesion molecule-1 (PECAM-1), vascular cell adhesion molecule-1 (VCAM-1), E-selectin, angiotensin-converting enzyme (ACE), aminopeptidase P (APP), plasmalemma vesicle protein-1 (PV1), P-selectin, VE-cadherin, receptors for cytokines, plasma proteins and microbes.

In certain embodiments, the targeting domain binds to ICAM-1, PECAM-1, VCAM-1, E-selectin, ACE, APP, PVA, P-selectin, VE-cadherin, cytokines, plasma proteins, and microbes, thereby directing the composition to the vasculature, including, but not limited to, the pulmonary vasculature or cerebral vasculature.

In one embodiment, the composition comprises a delivery vehicle conjugated to a targeting domain that binds ICAM-1 or PECAM-1, thereby directing the composition to the pulmonary vasculature. In one embodiment, the composition comprises a delivery vehicle conjugated to a targeting domain that binds VCAM-1, thereby directing the composition to the cerebral vasculature.

However, the present invention is not limited to vehicles directed to the cerebral vasculature or pulmonary vasculature. Rather, the present invention encompasses a delivery vehicle comprising a targeting domain that directs the vehicle to the vasculature or to any specific region of the vasculature, as mediated the by binding of the targeting domain to a specific marker. In some embodiments, the vehicle is targeted to a specific treatment site in need. For example, it is demonstrated herein that the targeting domain can be directed specifically to the inflamed states within the vasculature.

The present invention also relates in part to methods of treating conditions related to the vasculature in subjects in need thereof, the method comprising the administration of a composition including a delivery vehicle conjugated to a targeting domain.

In various embodiments, the invention provides a method for treating a pulmonary condition by targeting the composition to the pulmonary vasculature. Exemplary pulmonary conditions include, but are not limited to, acute lung injury, pulmonary ischemia including organ transplantation, pulmonary embolism, pulmonary edema, pulmonary hypertension, fibrosis, infection, inflammation, emphysema, and cancer.

In various embodiments, the invention provides a method for treating a neurological condition by targeting the composition to the cerebral vasculature. Exemplary neurological conditions include but not limited to, stroke, inflammation, infection, meningitis, traumatic brain injury, multiple sclerosis, concussion, cerebral embolism, hemorrhage, brain tumors, neurodegenerative disorders, depression, post-traumatic stress disorder, anxiety, mood disorders, and addiction disorders.

### Delivery Vehicle

In some embodiments, the delivery vehicle is a colloidal dispersion system, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the at least one agent into a host cell (in vitro, ex vivo or in vivo). In another aspect, the at least one agent may be associated with a lipid. The at least one agent associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/nucleic acid or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-agent complexes.

In one embodiment, delivery of the at least one agent comprises any suitable delivery method, including exemplary delivery methods described elsewhere herein. In certain embodiments, delivery of the at least one agent to a subject comprises mixing the at least one agent with a transfection reagent prior to the step of contacting. In another embodiment, a method of the present invention further comprises administering the at least one agent together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. In some embodiments, the liposomes comprise an internal aqueous space for entrapping water-soluble compounds. In another embodiment, liposomes can deliver the at least one agent to cells in an active form.

In one embodiment, the composition comprises a lipid nanoparticle (LNP) and at least one agent.

The term “lipid nanoparticle” refers to a particle having at least one dimension on the order of nanometers (e.g., 1-1,000 nm) which includes one or more lipids. In various embodiments, the particle includes a lipid of Formula (I), (II) or (III). In some embodiments, lipid nanoparticles are included in a formulation comprising at least one agent as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound IVa). In some embodiments, the at least one agent is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In one embodiment, the lipid nanoparticles have a mean diameter of about 83 nm. In one embodiment, the lipid nanoparticles have a mean diameter of about 102 nm. In one embodiment, the lipid nanoparticles have a mean diameter of about 103 nm. In some embodiments, the lipid nanoparticles are substantially non-toxic. In certain embodiments, the at least one agent, when present in the lipid nanoparticles, is resistant in aqueous solution to degradation by intra- or intercellular enzymes

The LNP may comprise any lipid capable of forming a particle to which the at least one agent is attached, or in which the at least one agent is encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

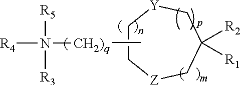

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;

R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;

R₅ is either absent or present and when present is hydrogen or C₁-C₆ alkyl;

m, n, and p are either the same or different and independently either 0 or 1 with the proviso that m, n, and p are not simultaneously 0;

q is 0, 1, 2, 3, or 4; and

Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid.

A representative useful dilinoleyl amino lipid has the formula:

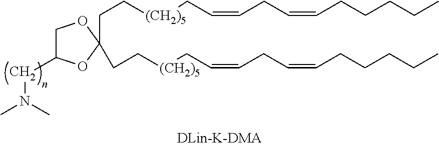

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):

(I)

or a pharmaceutically acceptable salt, tautomer, prodrug or stereoisomer thereof, wherein:

L¹ and L² are each independently —O(C═O)—, —(C═O)O— or a carbon-carbon double bond;

R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently methyl or cycloalkyl;

R⁷ is, at each occurrence, independently H or C₁-C₁₂ alkyl;

R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;

a and d are each independently an integer from 0 to 24;

b and c are each independently an integer from 1 to 24; and

e is 1 or 2.

In certain embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In certain embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —(C═O)O—. In other embodiments, both L¹ and L² are —(C═O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

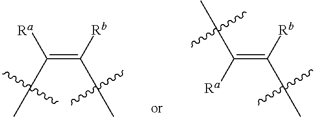

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

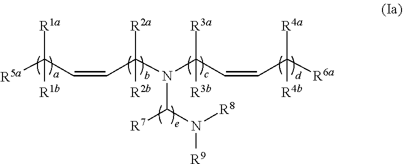

In other embodiments, the lipid compounds of Formula (I) have the following structure (Ib):

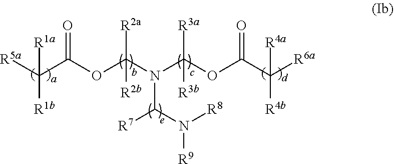

In yet other embodiments, the lipid compounds of Formula (I) have the following structure (Ic):

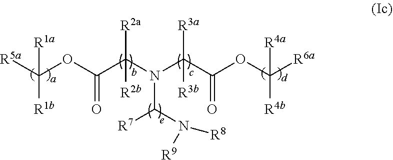

In certain embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In certain embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (I) are not particularly limited in the foregoing embodiments. In certain embodiments one or both of R⁵ or R⁶ is methyl. In certain other embodiments one or both of R⁵ or R⁶ is cycloalkyl for example cyclohexyl. In these embodiments the cycloalkyl may be substituted or not substituted. In certain other embodiments the cycloalkyl is substituted with C₁-C₁₂ alkyl, for example tert-butyl.

The substituents at R⁷ are not particularly limited in the foregoing embodiments of Formula (I). In certain embodiments at least one R⁷ is H. In some other embodiments, R⁷ is H at each occurrence. In certain other embodiments R⁷ is C₁-C₁₂ alkyl.

In certain other of the foregoing embodiments of Formula (I), one of R⁸ or R⁹ is methyl. In other embodiments, both R⁸ and R⁹ are methyl.

In some different embodiments of Formula (I), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, exemplary lipid of Formula (I) can include

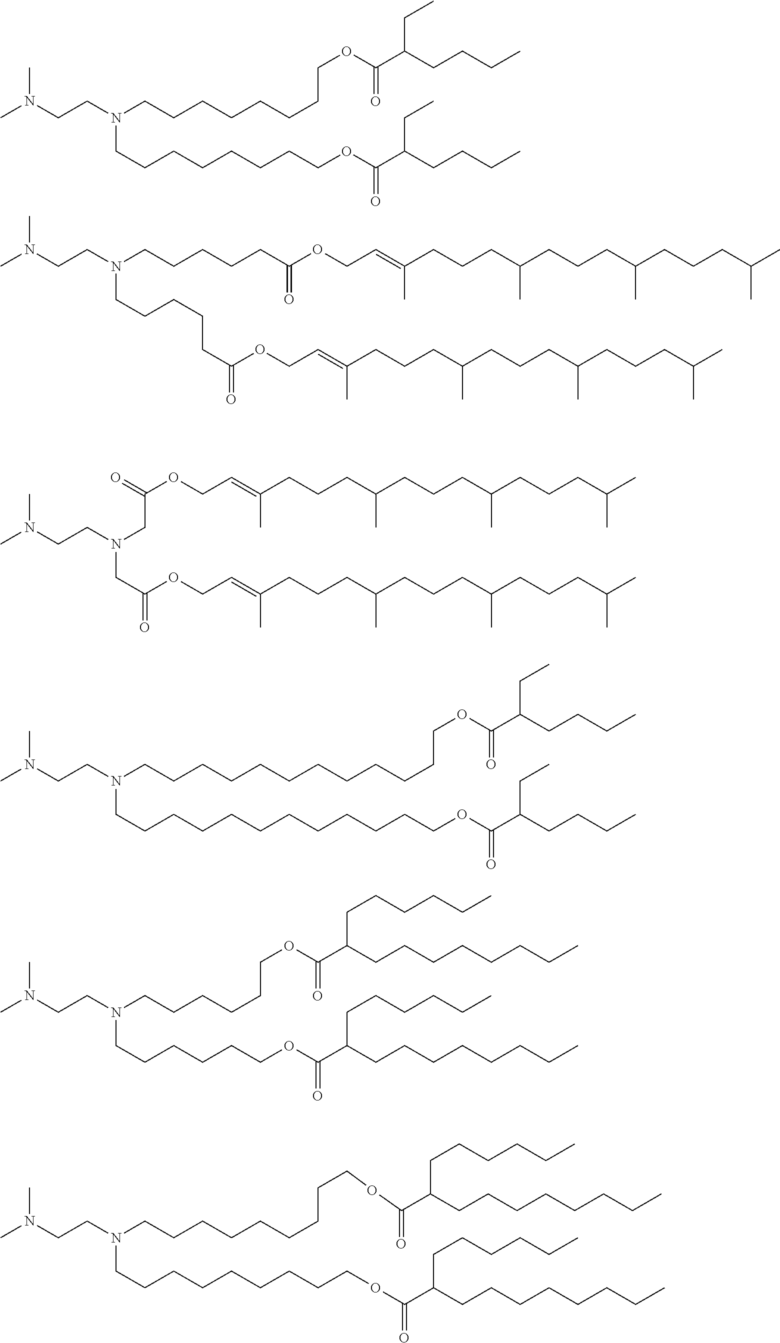

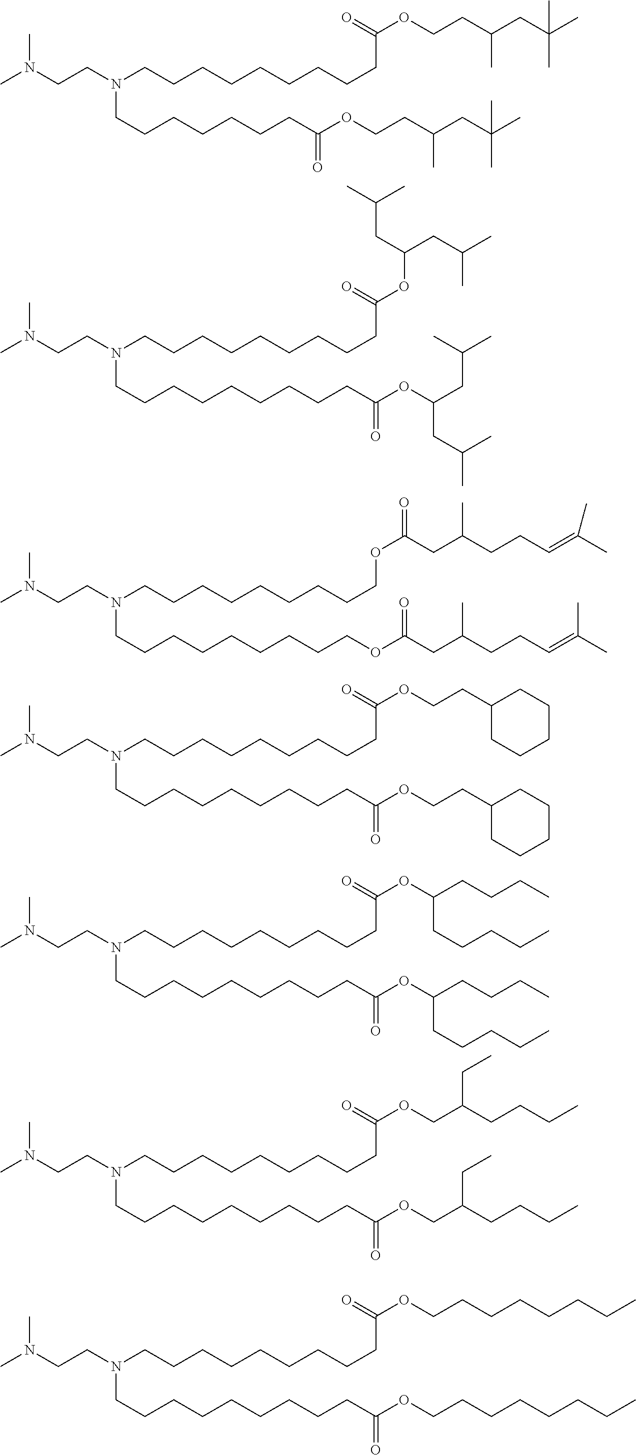

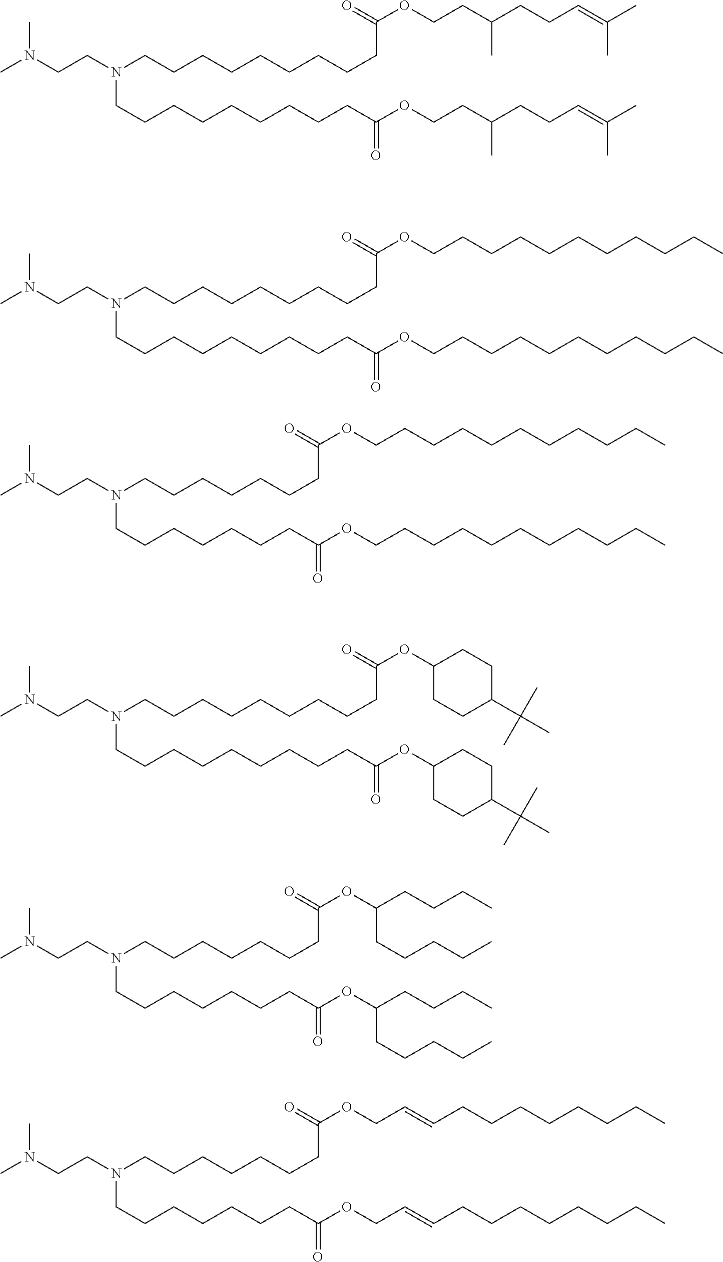

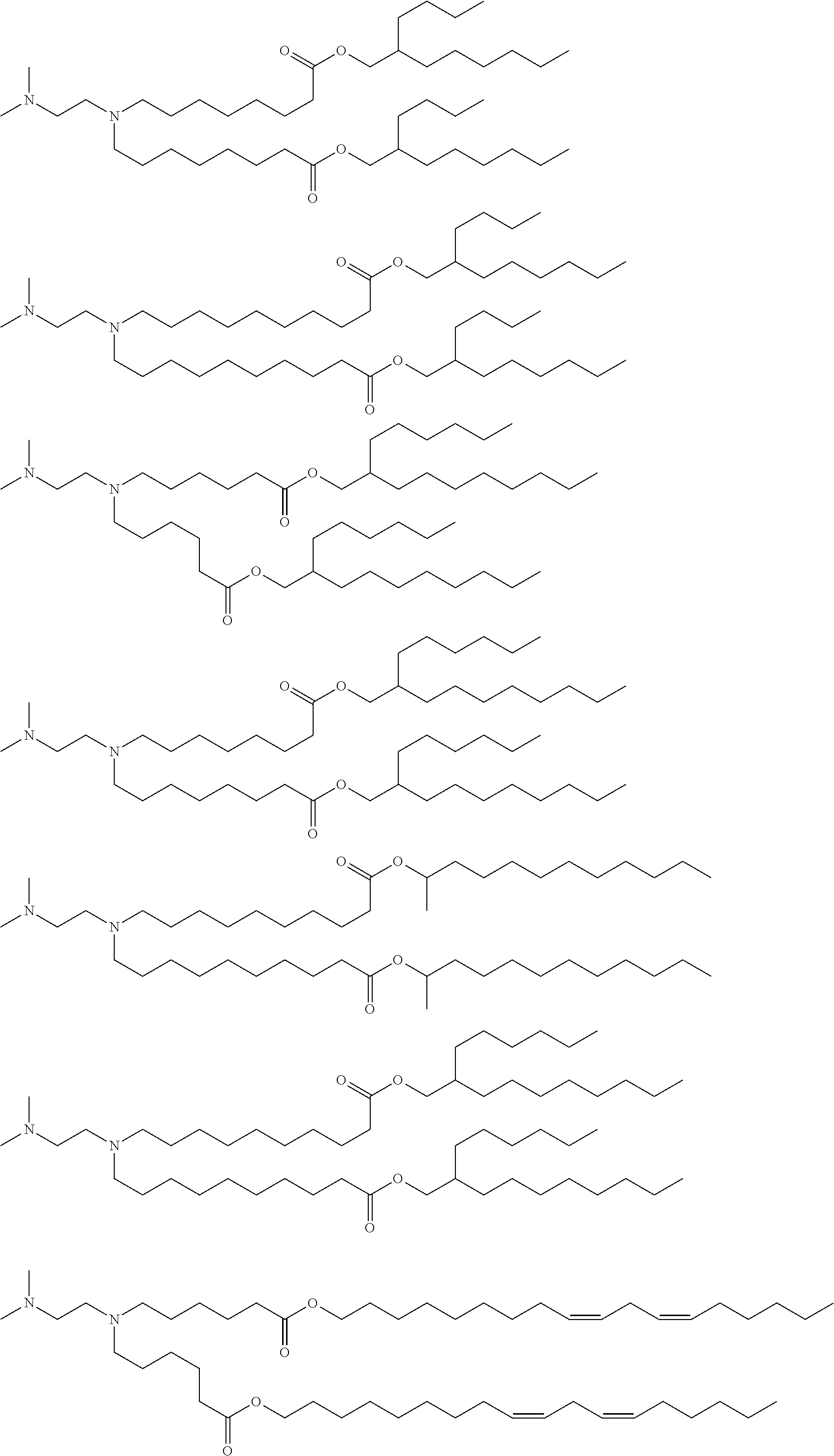

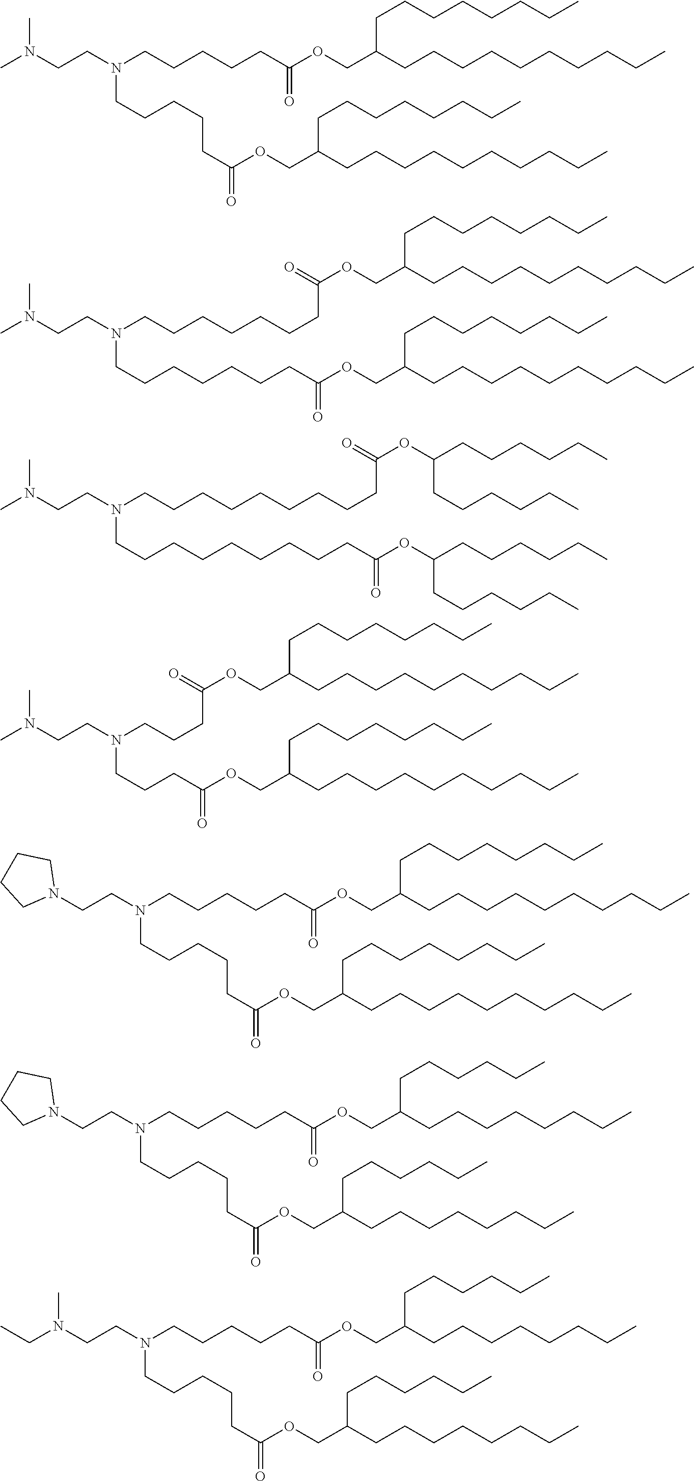

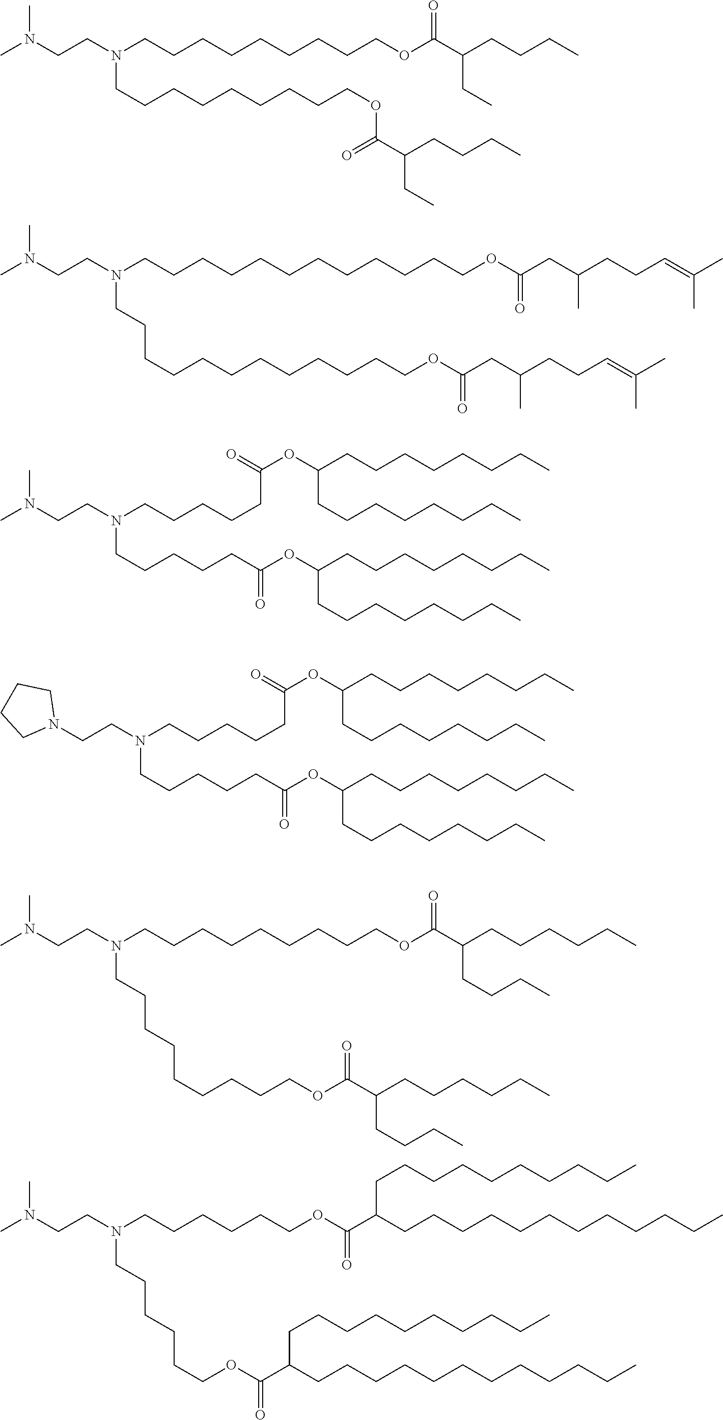

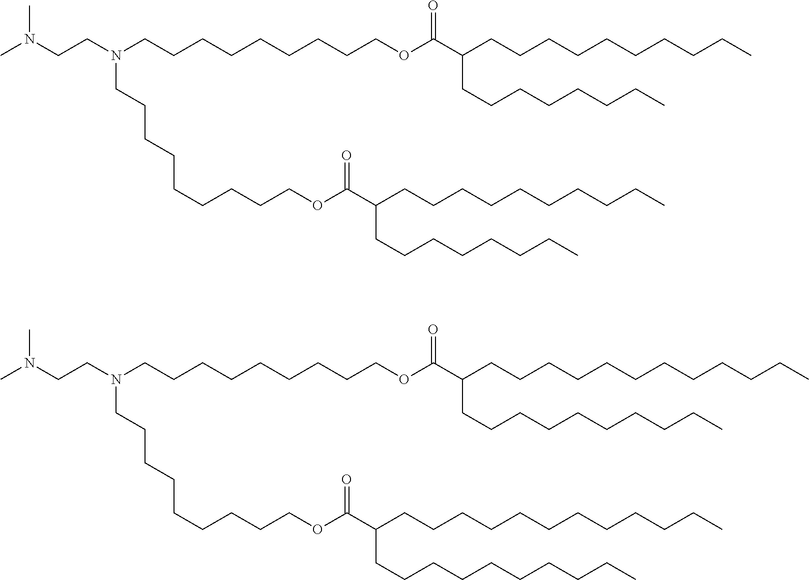

In some embodiments, the LNPs comprise a lipid of Formula (I), at least one agent, and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II:

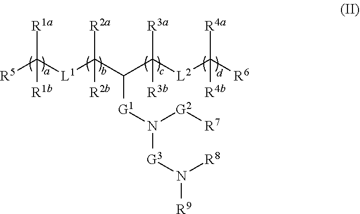

or a pharmaceutically acceptable salt, tautomer, prodrug or stereoisomer thereof, wherein:

L¹ and L² are each independently —O(C═O)—, —(C═O)O—, —C(O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;

G¹ is C₁-C₂ alkylene, —(C═O)—, —O(C═O)—, —SC(═O)—, —NR^{a}C(═O)— or a direct bond;

G² is —C(═O)—, —(C═O)O—, —C(═O)S—, —C(═O)NR^{a} or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either: (a) H or C₁-C₁₂ alkyl; or (b) R⁴ is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is C₄-C₂₀ alkyl;

R⁸ and R⁹ are each independently C₁-C₁₂ alkyl; or R⁸ and R⁹, together with the nitrogen atom to which they are attached, form a 5, 6 or 7-membered heterocyclic ring;

a, b, c and d are each independently an integer from 1 to 24; and

x is 0, 1 or 2.

In some embodiments of Formula (II), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

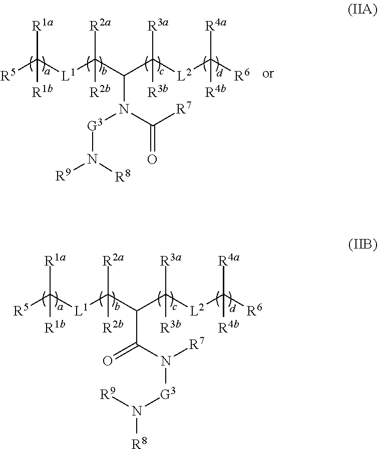

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L^{t} or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

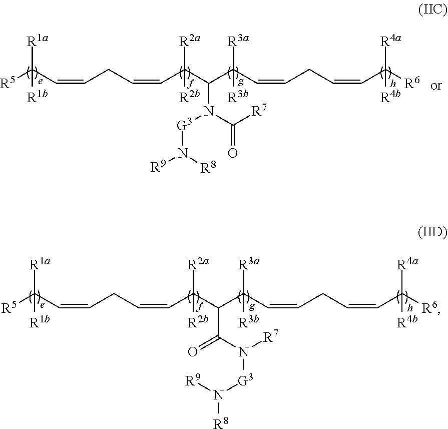

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In certain embodiments of Formula (II), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In certain embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In certain embodiments one of R⁵ or R⁶ is methyl. In other embodiments each of R⁵ or R⁶ is methyl.

The substituents at R⁷ of Formula (II) are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments R^{b} has one of the following structures:

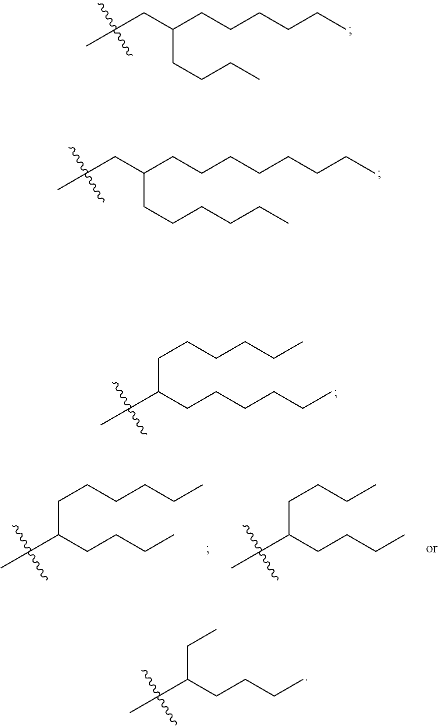

In certain other of the foregoing embodiments of Formula (II), one of R or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the following structures:

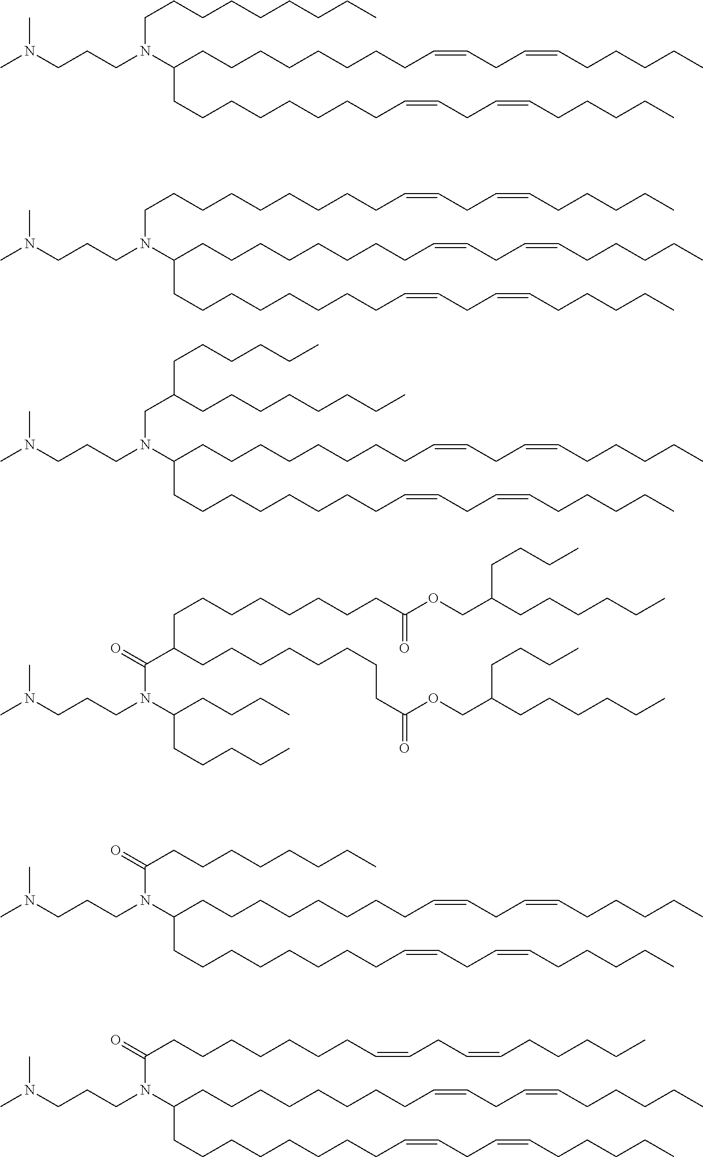

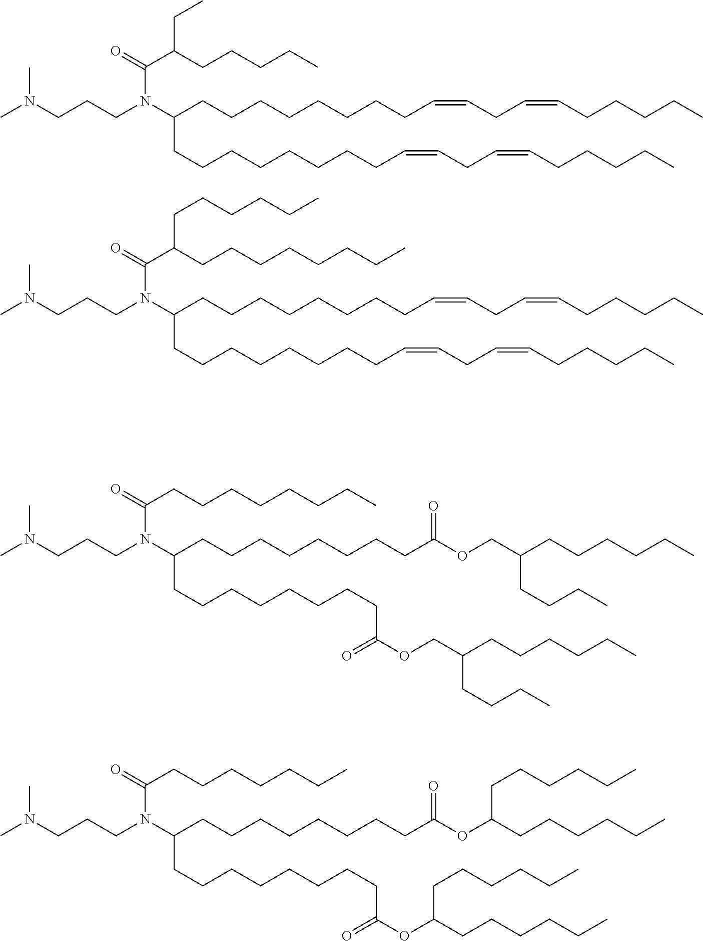

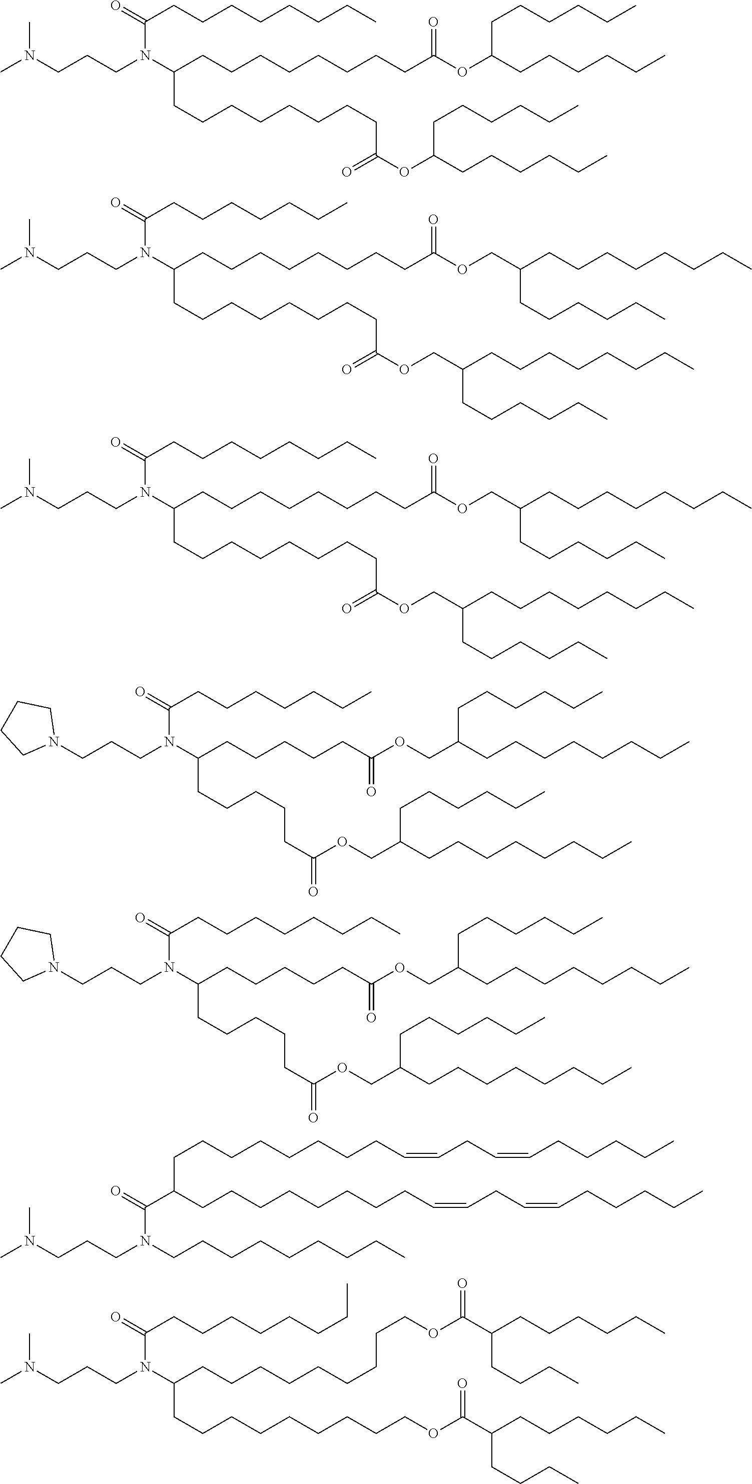

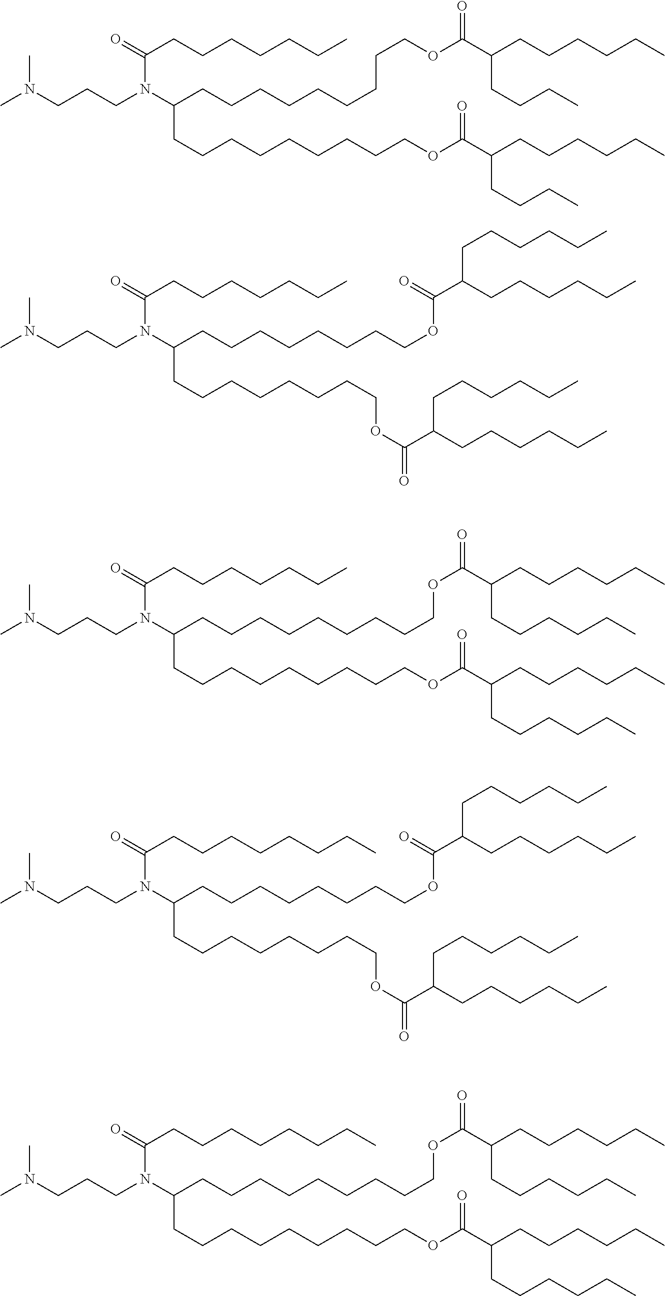

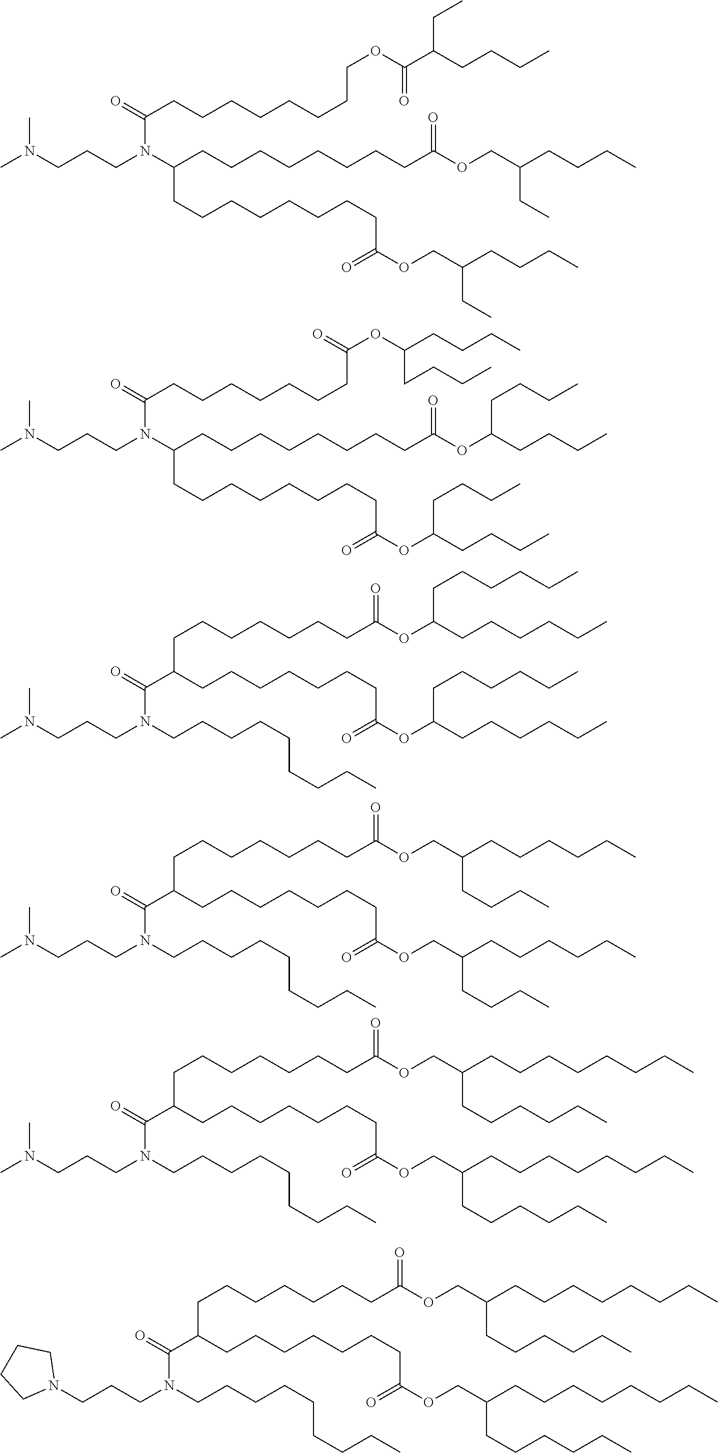

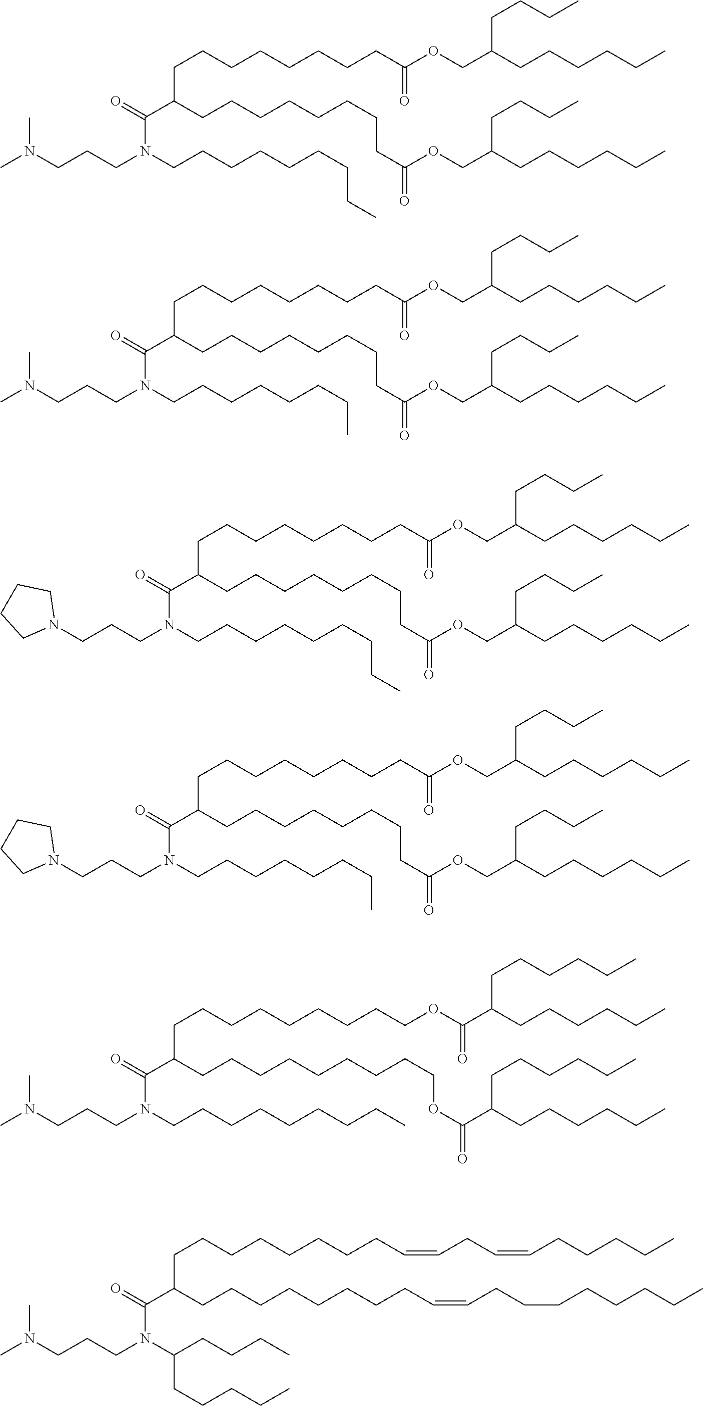

In some embodiments, the LNPs comprise a lipid of Formula (II), at least one agent, and one or more excipient selected from neutral lipids, steroids and pegylated lipids. In some embodiments, the lipid of Formula (II) is compound 11-9. In some embodiments, the lipid of Formula (II) is compound II-10. In some embodiments, the lipid of Formula (II) is compound II-11. In some embodiments, the lipid of Formula (II) is compound 11-12. In some embodiments, the lipid of Formula (II) is compound II-32.

In some other embodiments, the cationic lipid component of the LNPs has the structure of Formula (III):

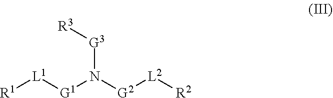

or a pharmaceutically acceptable salt, tautomer, prodrug or stereoisomer thereof, wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR—, —OC(═O)NR— or —NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —NR⁵C(═O)R⁴;

R⁴ is C₁-C₁₂ alkyl;

R⁵ is H or C₁-C₆ alkyl; and

x is 0, 1 or 2.

In some of the foregoing embodiments of Formula (III), the lipid has one of the following structures (IIIA) or (IIIB):

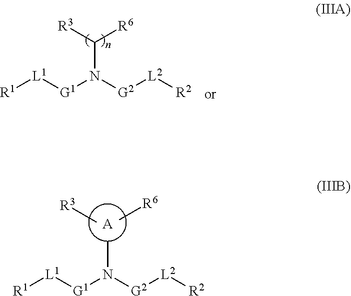

wherein:

A is a 3 to 8-membered cycloalkyl or cycloalkylene ring;

R⁶ is, at each occurrence, independently H, OH or C₁-C₂₄ alkyl;

n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

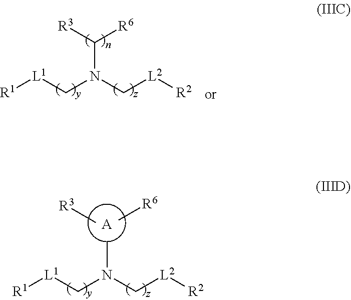

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

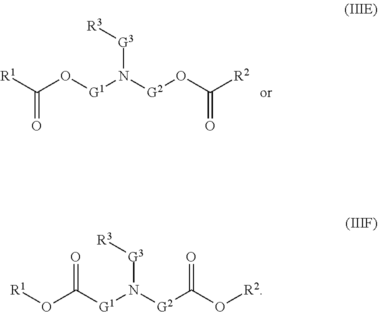

In some of the foregoing embodiments of Formula (III), the lipid has one of the following structures (IIIG), (IIIH), (IIII), or (IIIJ):

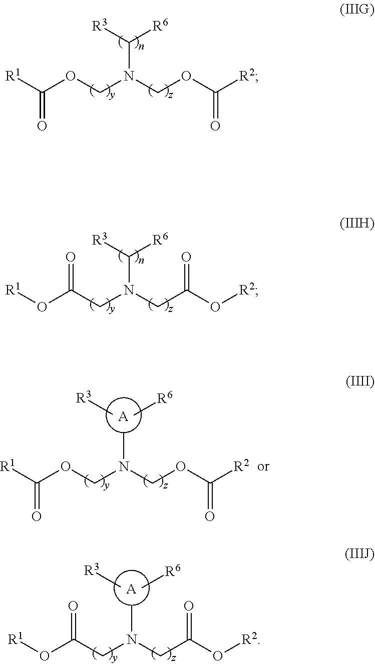

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

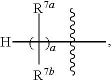

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

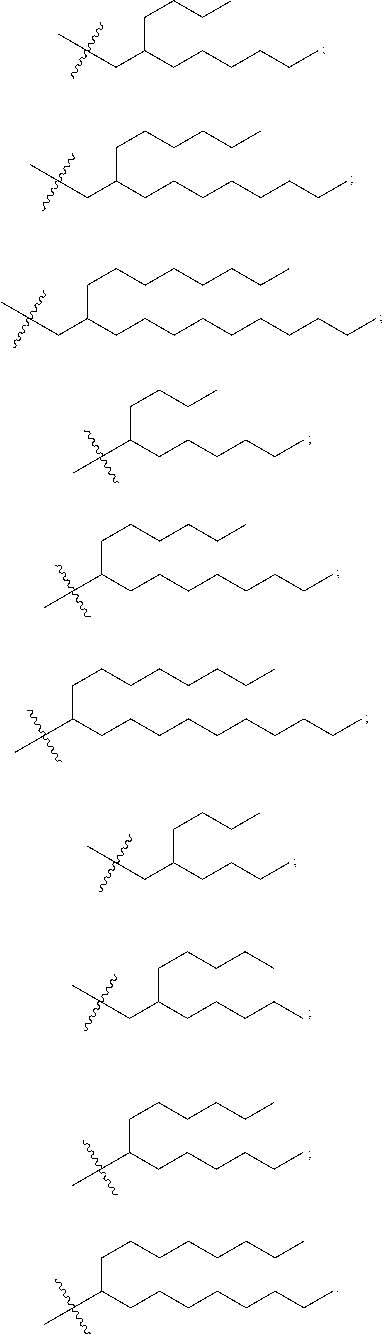

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the following structures:

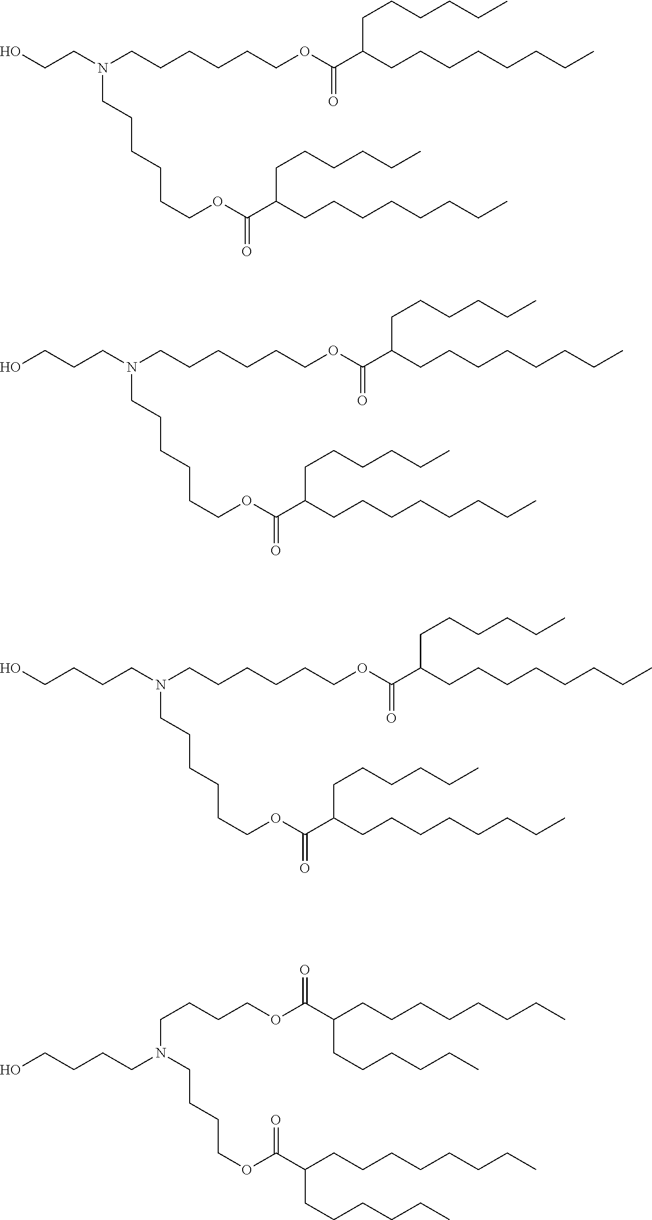 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC]

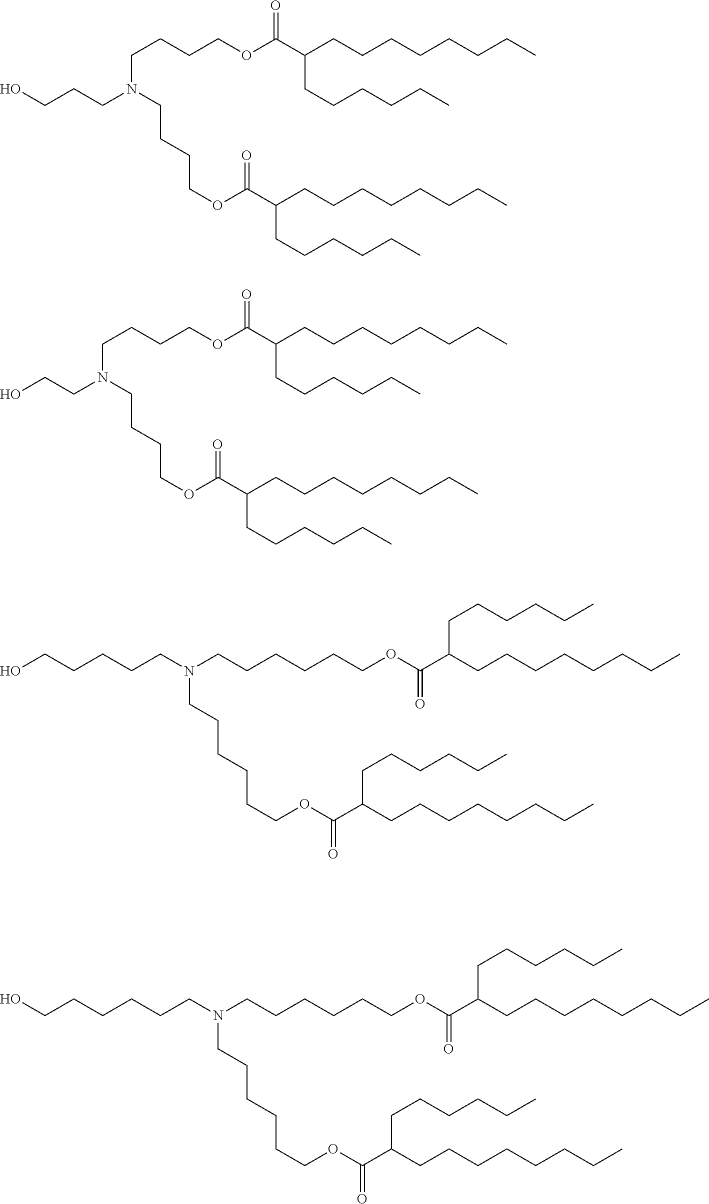 [SMILES: CCCCCCCCC(CCCCCC)C(=O)OCCCCN(CCCO)CCCCOC(=O)C(CCCCCC)CCCCCCCC ; CCCCCCCCC(CCCCCC)C(=O)OCCCCN(CCO)CCCCOC(=O)C(CCCCCC)CCCCCCCC ; CCCCCCCCC(CCCCCC)C(=O)OCCCCCCN(CCCCCO)CCCCCCOC(=O)C(CCCCCC)CCCCCCCC ; CCCCCCCCC(CCCCCC)C(=O)OCCCCCCN(CCCCCCO)CCCCCCOC(=O)C(CCCCCC)CCCCCCCC]

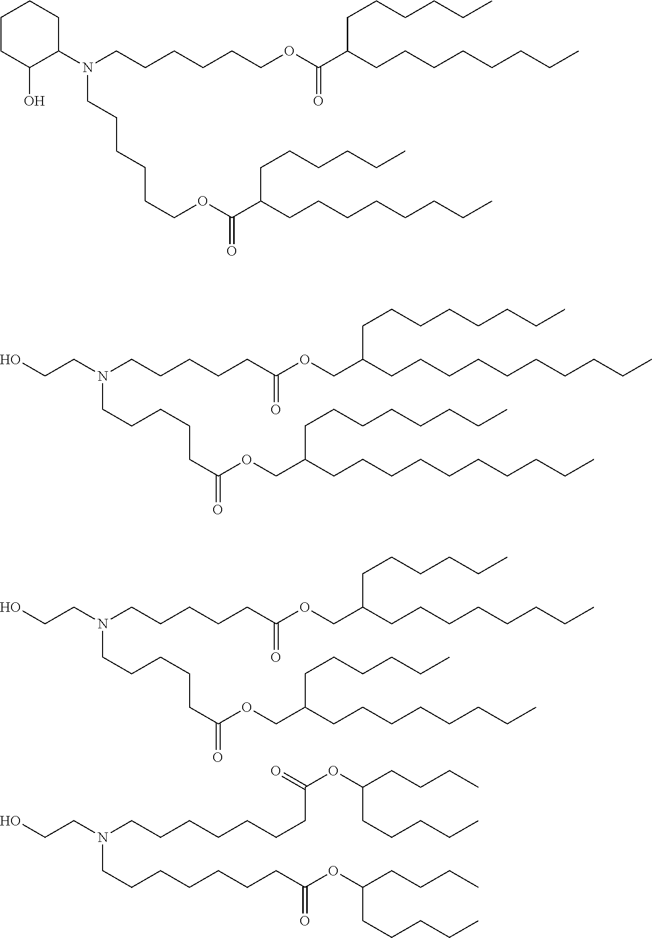 [SMILES: CCCCCCCCC(CCCCCC)C(=O)OCCCCCCN(CCCCCCOC(=O)C(CCCCCC)CCCCCCCC)C1CCCCC1O ; CCCCCCCCCCC(CCCCCCCC)COC(=O)CCCCCN(CCO)CCCCCC(=O)OCC(CCCCCCCC)CCCCCCCCCC ; CCCCCCCCC(CCCCCC)COC(=O)CCCCCN(CCO)CCCCCC(=O)OCC(CCCCCC)CCCCCCCC ; CCCCCC(CCCC)OC(=O)CCCCCCCN(CCO)CCCCCCCC(=O)OC(CCCC)CCCCC]

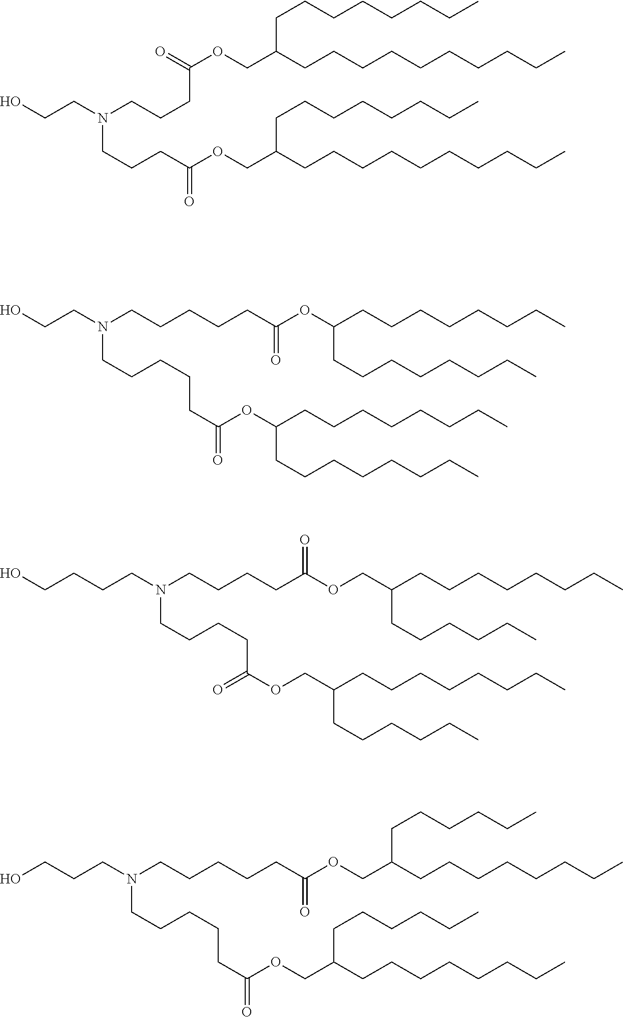 [SMILES: CCCCCCCCCCC(CCCCCCCC)COC(=O)CCCN(CCO)CCCC(=O)OCC(CCCCCCCC)CCCCCCCCCC ; CCCCCCCCC(CCCCCCCC)OC(=O)CCCCCN(CCO)CCCCCC(=O)OC(CCCCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)COC(=O)CCCCCN(CCCO)CCCCCC(=O)OCC(CCCCCC)CCCCCCCC]

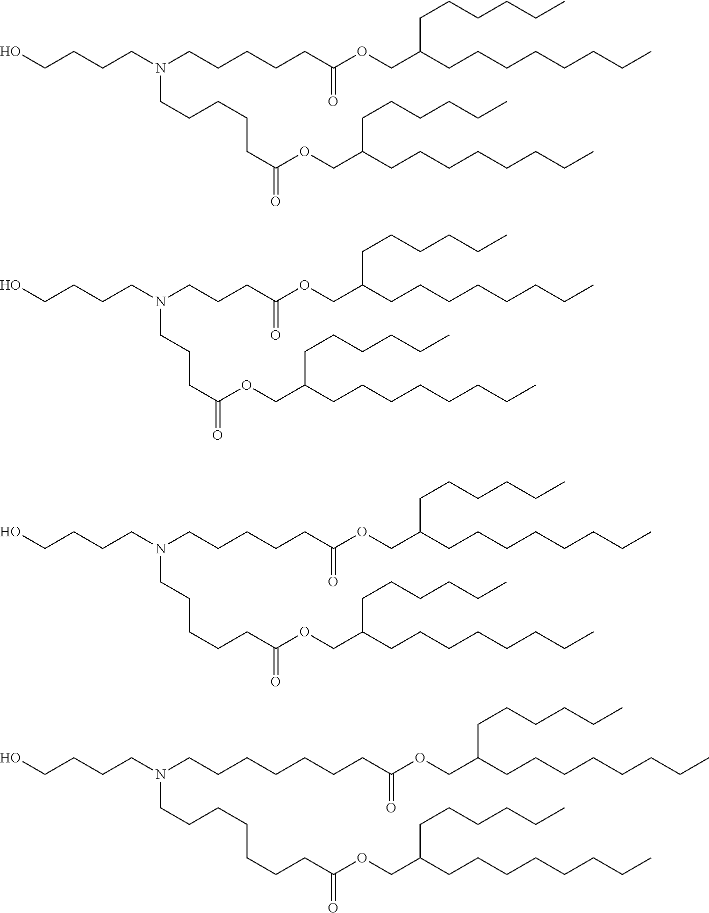 [SMILES: CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCN(CCCCO)CCCC(=O)OCC(CCCCCC)CCCCCCCC ; CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCCCCCN(CCCCO)CCCCCCCC(=O)OCC(CCCCCC)CCCCCCCC]

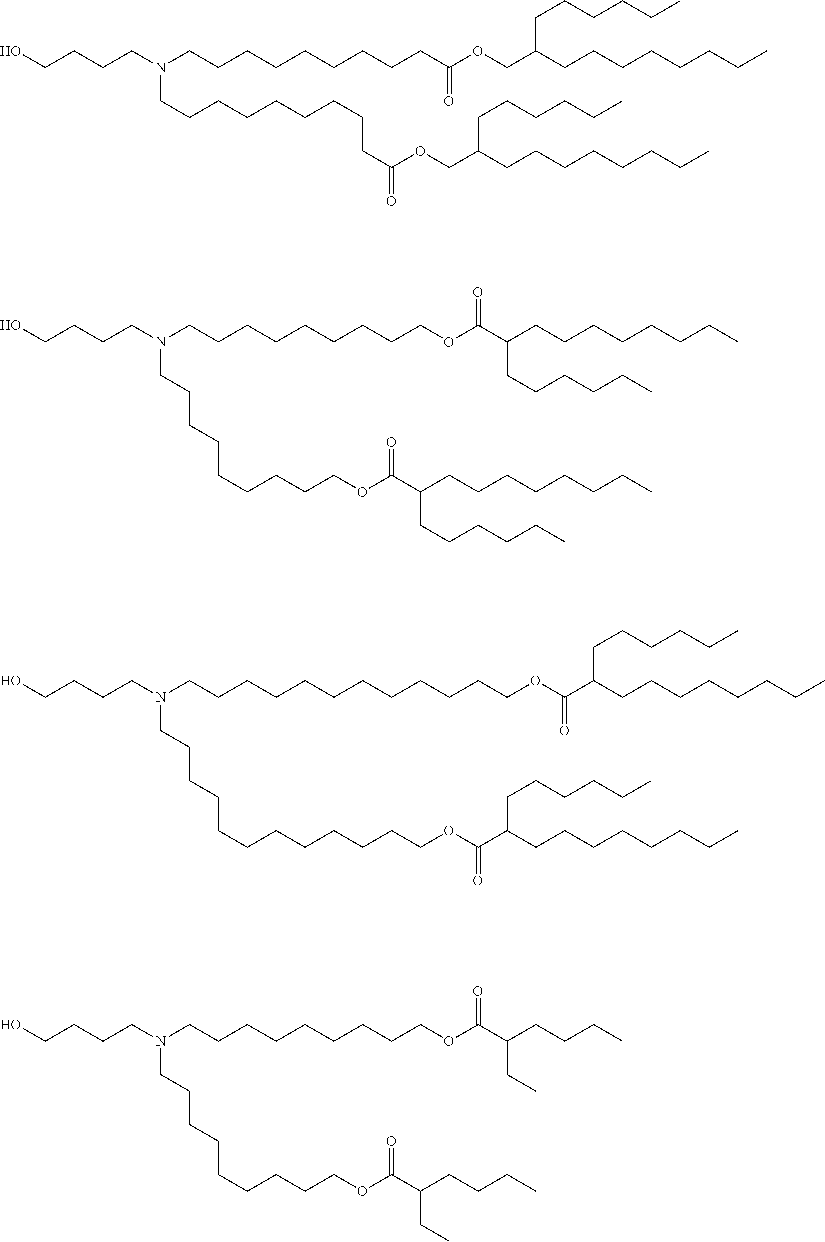 [SMILES: CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCCC ; CCCCC(CC)C(=O)OCCCCCCCCCN(CCCCO)CCCCCCCCCOC(=O)C(CC)CCCC]

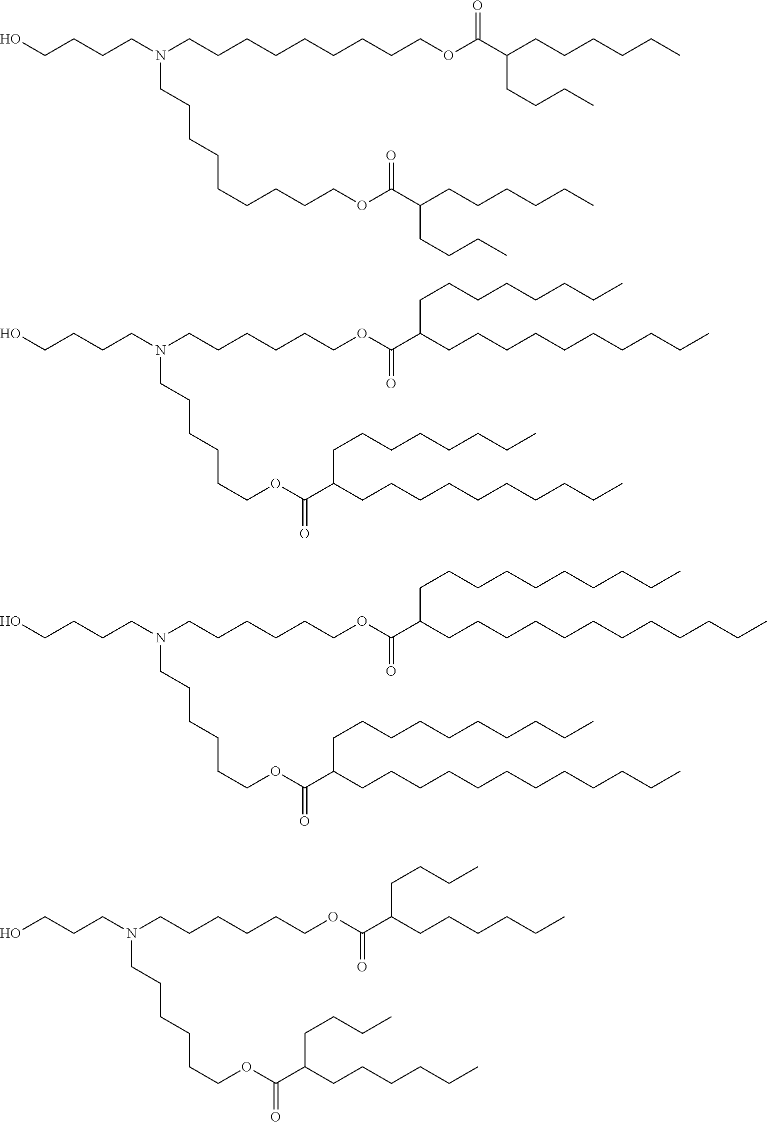

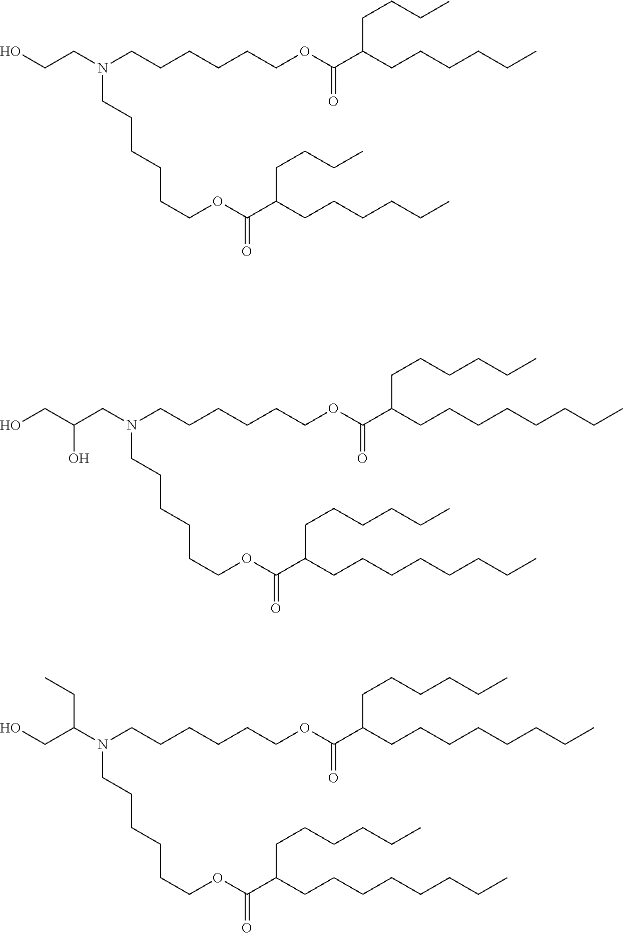 [SMILES: CCCCCCC(CCCC)C(=O)OCCCCCCN(CCO)CCCCCCOC(=O)C(CCCC)CCCCCC ; CCCCCCCCC(CCCCCC)C(=O)OCCCCCCN(CCCCCCOC(=O)C(CCCCCC)CCCCCCCC)CC(O)CO ; CCCCCCCCC(CCCCCC)C(=O)OCCCCCCN(CCCCCCOC(=O)C(CCCCCC)CCCCCCCC)C(CC)CO]

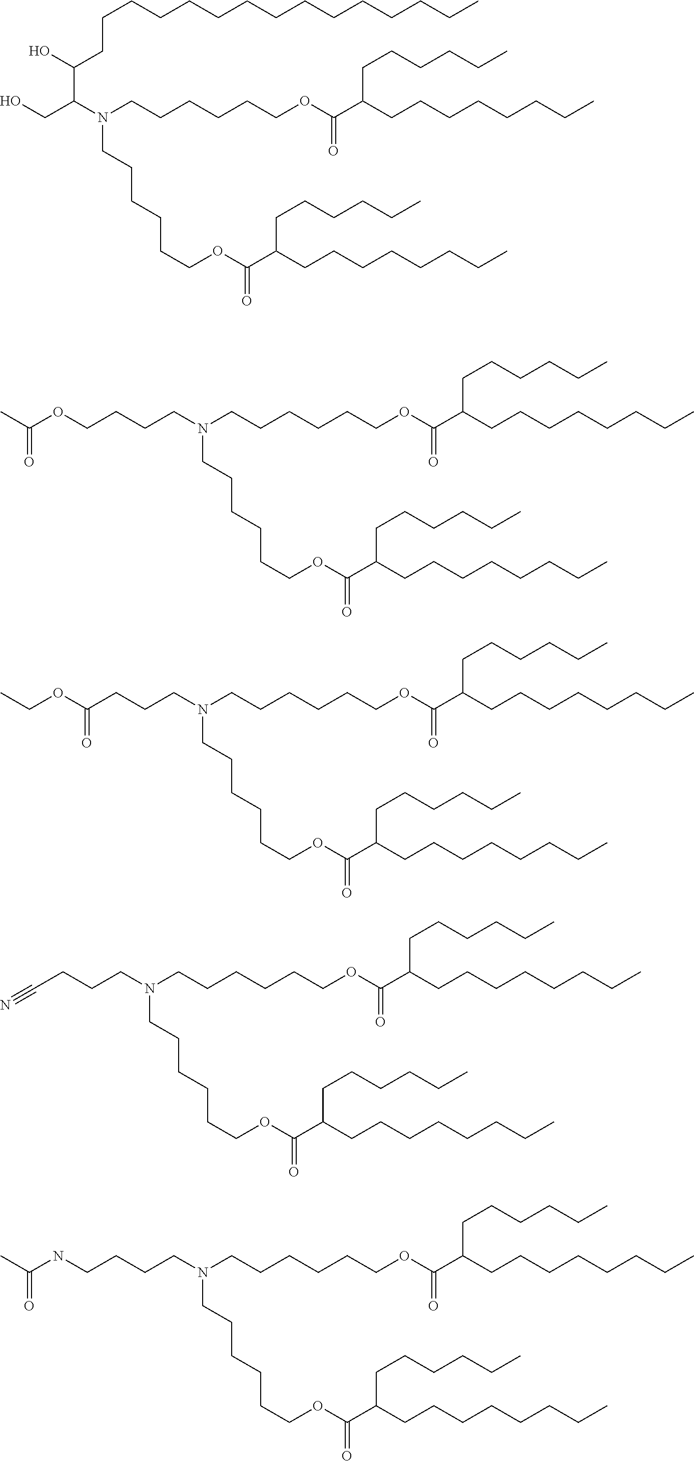

In some embodiments, the LNPs comprise a lipid of Formula (III), at least one agent, and one or more excipient selected from neutral lipids, steroids and pegylated lipids. In some embodiments, the lipid of Formula (III) is compound 111-3. In some embodiments, the lipid of Formula (III) is compound 111-7.

In certain embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In certain embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

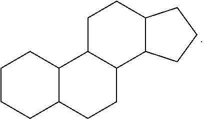 [SMILES: C1CCC2C(C1)CCC1C3CCCC3CCC21]

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GM₁). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing—lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O—(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

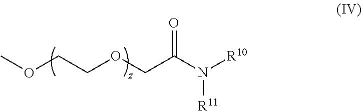

or a pharmaceutically acceptable salt, tautomer or stereoisomer thereof, wherein:

R¹⁰ and R¹¹ are each independently a straight or branched, saturated or unsaturated alkyl chain containing from 10 to 30 carbon atoms, wherein the alkyl chain is optionally interrupted by one or more ester bonds; and

z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

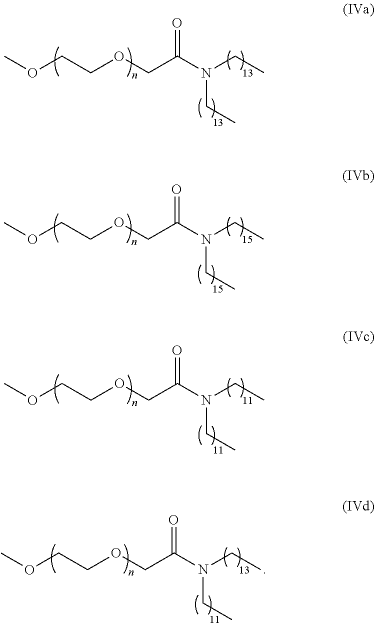

wherein n is an integer selected such that the average molecular weight of the pegylated lipid is about 2500 g/mol.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound 1-6. In different embodiments, the neutral lipid is DSPC. In other embodiments, the steroid is cholesterol. In still different embodiments, the pegylated lipid is compound IVa.

In certain embodiments, the LNP comprises one or more targeting moieties that targets the LNP to a cell or cell population. For example, in one embodiment, the targeting domain is a ligand which directs the LNP to a receptor found on a cell surface.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

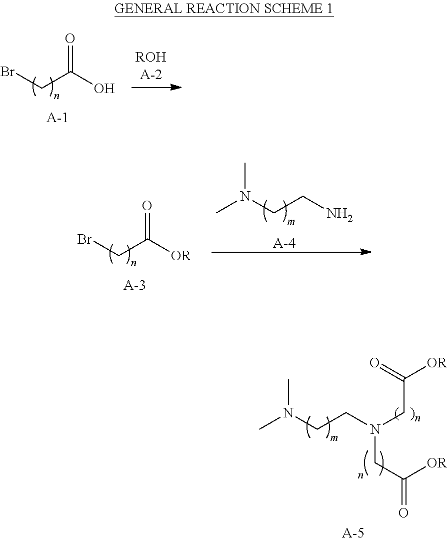

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

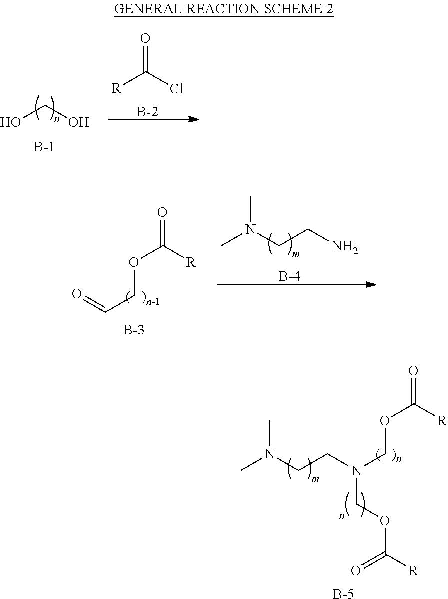

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

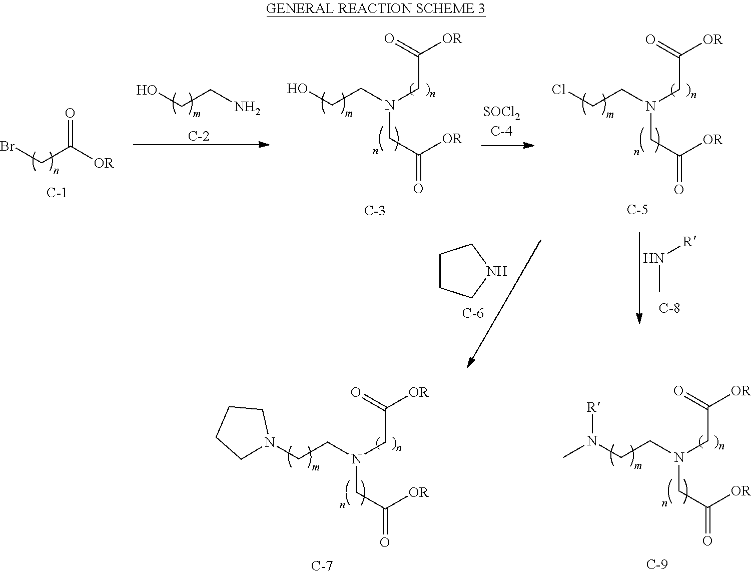

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

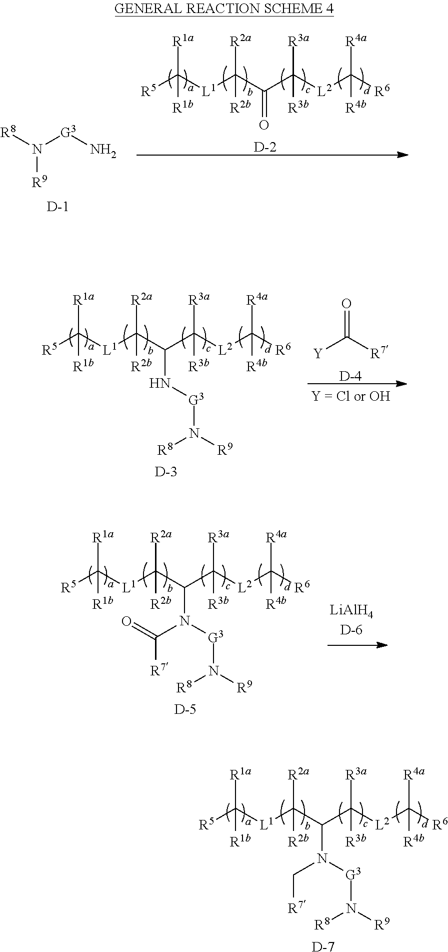

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAH4 D-6 to give D-7 after any necessary work up and/or purification.

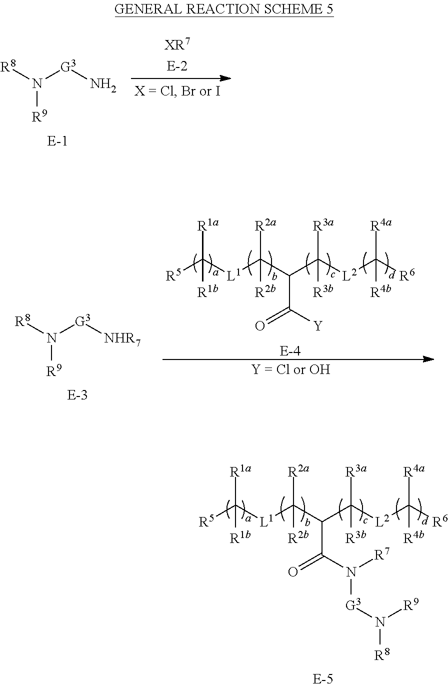

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

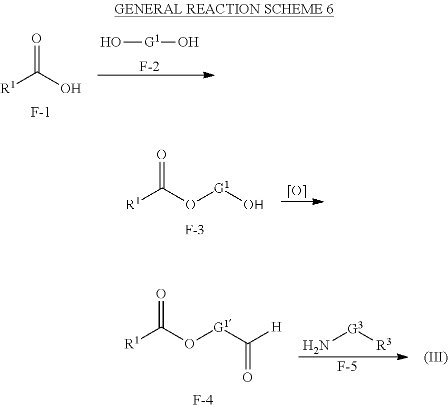

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Agents

In one embodiment, the delivery vehicle comprises at least one agent. In some embodiments, the agent is a therapeutic agent, an imaging agent, diagnostic agent, a contrast agent, a labeling agent, a detection agent, or a disinfectant. The agent may also include substances with biological activities which are not typically considered to be active ingredients, such as fragrances, sweeteners, flavorings and flavor enhancer agents, pH adjusting agents, effervescent agents, emollients, bulking agents, soluble organic salts, permeabilizing agents, anti-oxidants, colorants or coloring agents, and the like.

In one embodiment, the delivery vehicle comprises at least one therapeutic agent. The present invention is not limited to any particular therapeutic agent, but rather encompasses any suitable therapeutic agent that can be included within the delivery vehicle. Exemplary therapeutic agents include, but are not limited to, anti-viral agents, anti-bacterial agents, anti-oxidant agents, thrombolytic agents, chemotherapeutic agents, anti-inflammatory agents, immunogenic agents, antiseptics, anesthetics, analgesics, pharmaceutical agents, small molecules, peptides, nucleic acids, and the like.

Imaging Agents

In one embodiment, the delivery vehicle comprises an imaging agent. Imaging agents are materials that allow the delivery vehicle to be visualized after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting that may be used in the delivery vehicle are provided in the Handbook of Targeted delivery of Imaging Agents, Torchilin, ed. (1995) CRC Press, Boca Raton, Fla.

Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Imaging agents must reach their targets to be effective; thus, in some embodiments, an efficient uptake by cells is desirable. A rapid uptake may also be desirable to avoid the RES, see review in Allport and Weissleder, Experimental Hematology 1237-1246 (2001).

Further, imaging agents preferably should provide high signal to noise ratios so that they may be detected in small quantities, whether directly, or by effective amplification techniques that increase the signal associated with a particular target. Amplification strategies are reviewed in Allport and Weissleder, Experimental Hematology 1237-1246 (2001), and include, for example, avidin-biotin binding systems, trapping of converted ligands, probes that change physical behavior after being bound by a target, and taking advantage of relaxation rates. Examples of imaging technologies include magnetic resonance imaging, radionuclide imaging, computed tomography, ultrasound, and optical imaging.

Delivery vehicles as set forth herein may advantageously be used in various imaging technologies or strategies, for example by incorporating imaging agents into delivery vehicles. Many imaging techniques and strategies are known, e.g., see review in Allport and Weissleder, Experimental Hematology 1237-1246 (2001); such strategies may be adapted to use with delivery vehicles. Suitable imaging agents include, for example, fluorescent molecules, labeled antibodies, labeled avidin:biotin binding agents, colloidal metals (e.g., gold, silver), reporter enzymes (e.g., horseradish peroxidase), superparamagnetic transferrin, second reporter systems (e.g., tyrosinase), and paramagnetic chelates.

In some embodiments, the imaging agent is a magnetic resonance imaging contrast agent. Examples of magnetic resonance imaging contrast agents include, but are not limited to, 1,4,7,10-tetraazacyclododecane-N,N′,N″N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (DOTA) and derivatives thereof (see U.S. Pat. Nos. 5,188,816, 5,219,553, and 5,358,704). In some embodiments, the imaging agent is an X-Ray contrast agent. X-ray contrast agents already known in the art include a number of halogenated derivatives, especially iodinated derivatives, of 5-amino-isophthalic acid.

Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In one embodiment, the therapeutic agent is a prodrug. In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In one embodiment, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Nucleic Acid Therapeutic Agents

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In one embodiment, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In one embodiment, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one aspect of the invention, a targeted gene or protein, can be inhibited by way of inactivating and/or sequestering the targeted gene or protein. As such, inhibiting the activity of the targeted gene or protein can be accomplished by using a nucleic acid molecule encoding a transdominant negative mutant.

In one embodiment, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PTPN22 using RNAi technology.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, Calif.) or they may be genetically expressed from DNA encoding them.

In one embodiment, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agent comprises a Cas peptide or a nucleic acid molecule encoding a Cas peptide.

In one embodiment, the agent comprises a miRNA or a mimic of a miRNA. In one embodiment, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

In Vitro Transcribed RNA

In one embodiment, the composition of the invention comprises in vitro transcribed (IVT) RNA. In one embodiment, the composition of the invention comprises in vitro transcribed (IVT) RNA encoding a therapeutic protein. In one embodiment, the composition of the invention comprises IVT RNA encoding a plurality of therapeutic proteins.

In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is a therapeutic protein, as described elsewhere herein.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce or enhance an adaptive immune response in an organism. Preferred genes are genes which are useful for a short-term treatment, or where there are safety concerns regarding dosage or the expressed gene.

In various embodiments, a plasmid is used to generate a template for in vitro transcription of RNA which is used for transfection.

Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the RNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the RNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized RNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase RNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

Nucleoside-Modified RNA

In one embodiment, the composition of the present invention comprises a nucleoside-modified nucleic acid. In one embodiment, the composition of the invention comprises a nucleoside-modified RNA encoding a therapeutic protein.

For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Kariko et al., 2008, Mol Ther 16:1833-1840; Kariko et al., 2012, Mol Ther 20:948-953). The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39:e142; Kariko et al., 2012, Mol Ther 20:948-953; Kariko et al., 2005, Immunity 23:165-175).

It has been demonstrated that the presence of modified nucleosides, including pseudouridines in RNA suppress their innate immunogenicity (Kariko et al., 2005, Immunity 23:165-175). Further, protein-encoding, in vitro-transcribed RNA containing pseudouridine can be translated more efficiently than RNA containing no or other modified nucleosides (Kariko et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Kariko et al., 2011, Nucleic Acids Research 39:e142). Administering HPLC-purified, pseudourine-containing RNA coding for erythropoietin into mice and macaques resulted in a significant increase of serum EPO levels (Kariko et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid, wherein the nucleic acid comprises a pseudouridine or a modified nucleoside. In certain embodiments, the composition comprises a vector, comprising an isolated nucleic acid, wherein the nucleic acid comprises a pseudouridine or a modified nucleoside.

In one embodiment, the nucleoside-modified RNA of the invention is IVT RNA, as described elsewhere herein. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N^{2,7}-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

Polypeptide Therapeutic Agents

In other related aspects, the therapeutic agent includes an isolated peptide that modulates a target. For example, in one embodiment, the peptide of the invention inhibits or activates a target directly by binding to the target thereby modulating the normal functional activity of the target. In one embodiment, the peptide of the invention modulates the target by competing with endogenous proteins. In one embodiment, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Antibody Therapeutic Agents

The invention also contemplates a delivery vehicle comprising an antibody, or antibody fragment, specific for a target. That is, the antibody can inhibit a target to provide a beneficial effect.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Combinations

In one embodiment, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

### Conjugation

In various embodiments of the invention, the delivery vehicle is conjugated to a targeting domain. Exemplary methods of conjugation can include, but are not limited to, covalent bonds, electrostatic interactions, and hydrophobic (“van der Waals”) interactions. In one embodiment, the conjugation is a reversible conjugation, such that the delivery vehicle can be disassociated from the targeting domain upon exposure to certain conditions or chemical agents. In another embodiment, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In one embodiment, the activated polymer conjugated lipid comprises a first coupling group capable of reacting with a second coupling group. In one embodiment, the activated polymer conjugated lipid is an activated pegylated lipid. In one embodiment, the first coupling group is bound to the lipid portion of the pegylated lipid. In another embodiment, the first coupling group is bound to the polyethylene glycol portion of the pegylated lipid. In one embodiment, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In one embodiment, the first coupling group is a maleimide.

In one embodiment, the second coupling group is a sulfhydryl group. The sulfhydryl group can be installed on the targeting domain using any method known to those of skill in the art. In one embodiment, the sulfhydryl group is present on a free cysteine residue. In one embodiment, the sulfhydryl group is revealed via reduction of a disulfide on the targeting domain, such as through reaction with 2-mercaptoethylamine. In one embodiment, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### Targeting Domain

In one embodiment, the composition comprises a targeting domain that directs the delivery vehicle to a site. In one embodiment, the site is a site in need of the agent comprised within the delivery vehicle. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In various embodiments, the targeting domain binds to a cell surface molecule of a vascular endothelial cell. Exemplary cell surface molecules, include but is not limited to, ICAM-1, PECAM-1, VCAM-1, ACE, APP, PV1, P-selectin, E-selectin, and VE-cadherin. In various embodiments, the targeting domain binds to a cell surface molecule of a vascular endothelial cell that is upregulated during inflammation or endothelial activation.

Peptides

In one embodiment, the targeting domain of the invention comprises a peptide. In certain embodiments, the peptide targeting domain specifically binds to a target of interest.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

Nucleic Acids

In one embodiment, the targeting domain of the invention comprises an isolated nucleic acid, including for example a DNA oligonucleotide and a RNA oligonucleotide. In certain embodiments, the nucleic acid targeting domain specifically binds to a target of interest. For example, in one embodiment, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

Antibodies

In one embodiment, the targeting domain of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

In one embodiment, the targeting domain of the instant invention is an antibody that specifically binds to endothelial cells lining vascular lumen. Exemplary targets include, but are not limited to, intercellular adhesion molecule-1 (ICAM-1), platelet-endothelial cell adhesion molecule-1 (PECAM-1), vascular cell adhesion molecule-1 (VCAM-1), angiotensin-converting enzyme (ACE), aminopeptidase P (APP), plasmalemma vesicle protein-1 (PV1), P-selectin, E-selectin, VE-cadherin, and receptors for cytokines, plasma proteins and microbes.

In one embodiment, the targeting domain is an antibody which specifically binds to ICAM-1. In one embodiment, the targeting domain is an antibody which specifically binds to PECAM-1. In one embodiment, the targeting domain is an antibody which specifically binds to VCAM-1. In one embodiment, the targeting domain is an antibody which specifically binds to ACE. In one embodiment, the targeting domain is an antibody which specifically binds to APP. In one embodiment, the targeting domain is an antibody which specifically binds to PV1.

Exemplary antibodies or antibody fragments that bind to an endothelial cell marker described herein and thus may be used as a targeting domain are well known in the art. An exemplary antibody that binds to PECAM-1 is Ab62 (Centocor). An exemplary antibody that binds to PECAM-1 are those produced from hybridoma clones clone 390. An exemplary antibody that binds to ICAM includes those produced from hybridoma clones of YN1/1.7.4 (ATCC® CRL-1878™). An exemplary antibody that binds to VCAM includes those produced from hybridoma clones of M/K-2.7 (ATCC® CRL-1909™).

### Therapeutic Methods

The present invention also provides methods of delivering at least one agent to endothelial cells lining vascular lumen. In certain embodiments, the method is used to treat or prevent a disease or disorder in a subject associated with inflammation, such as in the brain or in the lung. In certain embodiments, the method is used to treat or prevent a disease or disorder in a subject associated with inflammation in the brain. Exemplary diseases or disorders include, but are not limited to, stroke, inflammation, infection, meningitis, traumatic brain injury, multiple sclerosis, concussion, cerebral embolism, hemorrhage, brain tumors, neurodegenerative disorders, depression, post-traumatic stress disorder, anxiety, mood disorders, and addiction disorders.

In certain embodiments, the method is used to treat or prevent a disease or disorder in a subject associated with inflammation in the lungs. Exemplary diseases or disorders include, but are not limited to, acute lung injury, pulmonary ischemia including organ transplantation, pulmonary embolism, pulmonary edema, pulmonary hypertension, fibrosis, infection, inflammation, emphysema, and cancer.

It will be appreciated by one of skill in the art, when armed with the present disclosure including the methods detailed herein, that the invention is not limited to treatment of diseases or disorders that are already established. Particularly, the disease or disorder need not have manifested to the point of detriment to the subject; indeed, the disease or disorder need not be detected in a subject before treatment is administered. That is, significant signs or symptoms of diseases or disorders do not have to occur before the present invention may provide benefit. Therefore, the present invention includes a method for preventing diseases or disorders, in that a composition, as discussed previously elsewhere herein, can be administered to a subject prior to the onset of diseases or disorders, thereby preventing diseases or disorders.

One of skill in the art, when armed with the disclosure herein, would appreciate that the prevention of a disease or disorder, encompasses administering to a subject a composition as a preventative measure against the development of, or progression of, a disease or disorder. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

The invention encompasses delivery of a delivery vehicle, comprising at least one agent, conjugated to a targeting domain. To practice the methods of the invention; the skilled artisan would understand, based on the disclosure provided herein, how to formulate and administer the appropriate composition to a subject. The present invention is not limited to any particular method of administration or treatment regimen.

One of skill in the art will appreciate that the compositions of the invention can be administered singly or in any combination. Further, the compositions of the invention can be administered singly or in any combination in a temporal sense, in that they may be administered concurrently, or before, and/or after each other. One of ordinary skill in the art will appreciate, based on the disclosure provided herein, that the compositions of the invention can be used to prevent or to treat a disease or disorder, and that a composition can be used alone or in any combination with another composition to affect a therapeutic result. In various embodiments, any of the compositions of the invention described herein can be administered alone or in combination with other modulators of other molecules associated with diseases or disorders.

In one embodiment, the invention includes a method comprising administering a combination of compositions described herein. In certain embodiments, the method has an additive effect, wherein the overall effect of the administering a combination of compositions is approximately equal to the sum of the effects of administering each individual inhibitor. In other embodiments, the method has a synergistic effect, wherein the overall effect of administering a combination of compositions is greater than the sum of the effects of administering each individual composition.

The method comprises administering a combination of composition in any suitable ratio. For example, in one embodiment, the method comprises administering two individual compositions at a 1:1 ratio. However, the method is not limited to any particular ratio. Rather any ratio that is shown to be effective is encompassed.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1

Endothelial cells lining vascular lumen represent targets for pharmacological interventions in many cardiovascular, neurological and pulmonary conditions (Shuvaev, et al., J. Control. Release 2015, 219, 576-595; Aird, Blood 2003, 101, 3765-3777; Maniatis, & Orfanos, Curr. Opin. Crit. Care 2008, 14, 22-30; Thorpe, Clin. Cancer Res. 2004, 10, 415-427). Endothelial targeting of diverse agents and carriers to the pulmonary, cerebrovascular and other vascular areas has been achieved using antibodies and other affinity ligands binding to intercellular adhesion molecule-1 (ICAM-1), platelet-endothelial cell adhesion molecule-1 (PECAM-1), vascular cell adhesion molecule-1 (VCAM-1), E-selectin, angiotensin-converting enzyme (ACE), aminopeptidase P (APP), and plasmalemma vesicle protein-1 (PV1) (Han, et al., Ther. Deliv. 2012, 3, 263-276; Howard, et al. ACS Nano 2014, 8, 4100-4132; Spragg, et al., Med. Sci. 1997, 94, 8795-8800; Nowak, et al., Eur. J. Cardio-thoracic Surg. 2010, 37, 859-863; Khoshnejad, et al., Bioconjug Chem 2016, 27, 628-637; Albelda, Am. J. Respir. Cell Mol. Biol. 1991, 4, 195-203).

Described herein is the development of a vascular targeting mRNA delivery platform. The experiments described herein were performed to investigate the efficacy of the targeting platform and its impact on directing biodistribution, fate, and specific activity of mRNA cargo to vascular endothelium. Pulmonary endothelial targeting ligands, anti-PECAM-1 and anti-ICAM-1 were conjugated to LNPs containing reporter mRNAs and evaluated in vitro and in vivo using various models. To demonstrate the utility of this system for targeting other organs including ones in pathological state, the current platform was validated in a local brain injury model. Inflammation and endothelial activation are recognized as very early events in a variety of CNS diseases, such as bacterial meningitis (Woehrl, et al., J. Infect. Dis. 2012, 202, 1389-96), multiple sclerosis (Larochelle, et al., FEBS Lett. 2011, 585, 3770-3780), and as secondary injuries in others, such as stroke, ischemia, and traumatic brain injury (TBI) (Perez-de-Puig, et al., Acta Neuropathol. 2015, 129, 239-257; Lutton, et al., Sci. Rep. 2017, 7, 3846). Taking advantage of upregulation of CAMs upon endothelial activation, the targeted platform decorated with anti-VCAM-1 antibodies was tested and the feasibility of targeted-LNP-mRNA to transfect cerebrovasculature was demonstrated. Importantly, selective delivery to an inflamed state was demonstrated.

The design of a successful targeting platform establishes a new venue for the development of RNA therapeutics for disorders in need of novel site-specific therapeutics, namely severe pathological cardiopulmonary and cerebrovascular conditions.

The materials and methods used in these experiments are now described.

### Reagents

N-succinimidyl S-acetylthioacetate (SATA) was purchased from Pierce Biotechnology (Rockford, Ill.). Radioactive isotope ¹²⁵I was purchased from Perkin-Elmer (Wellesley, Mass.). Whole molecule rat IgG was from ThermoFisher (Waltham, Mass.). Anti-mouse-PECAM/CD31 monoclonal antibody was obtained from BioLegend (San Diego, Calif.). Monoclonal antibodies to human PECAM-1 (anti-PECAM, Ab62) were provided (Centocor) (Han, et al., J Control Release 2015, 210, 39-47). Anti-human ICAM, and anti-mouse VCAM were produced in-house from the hybridoma clones of YN1/1.7.4 (ATCC® CRL-1878™) and M/K-2.7 (ATCC® CRL-1909™), respectively. All chemical reagents were purchased from Sigma Aldrich unless stated otherwise.

### Cell Culture

Human mesothelioma REN cells, either stably expressing human PECAM-1 (REN-PECAM) or PECAM-1-negative cells (REN wild type), have been previously described (Garnacho, et al., Blood 2008, 111, 3024-3033). REN cells were maintained in RPMI 1640 supplemented with 10% FBS, 2 mM L-glutamine, 100 U/mL penicillin, and 100 μg/mL streptomycin (Life Technologies, Carlsbad, Calif.). Maintenance media for REN-PECAM cells also contained Geneticin (G418) at 200 μg/mL, as a selection antibiotic.

Human umbilical vein endothelial cells (HUVECs), purchased at passage 1 from Lonza (Walkersville, Md.) and subcultured up to four passages in endothelial basal medium (EBM) supplemented with EGM-bulletkit (Lonza). Passages between 4 and 6 were used throughout the studies.

mRNA Production and Formulation into Lipid Nanoparticles

mRNAs were produced as described previously (Pardi, et al., Nat. Commun. 2017, 8, 14630; Pardi, et al., Nature 2017, 543, 248-251) using T7 RNA polymerase (Megascript, Ambion) on linearized plasmids encoding codon-optimized firefly luciferase (pLuc19) and eGFP (pTEV-eGFP-A101). To make modified nucleoside-containing mRNA, m1Ψ-5′-triphosphate (TriLink) was incorporated instead of UTP. mRNAs were transcribed to contain 110 (pLuc19) or 101 (pTEV-eGFP-A101) nt poly(A) tails. They were capped using the m7G capping kit with 2′-O-methyltransferase (ScriptCap, CellScript) to obtain cap1. mRNA was purified by Fast Protein Liquid Chromatography (FPLC) (Akta Purifier, GE Healthcare). All prepared RNAs were analyzed by electrophoresis using denaturing or native agarose gels, and stored at −20° C. LNPs used in this study were similar in composition to those described elsewhere (Pardi, et al., Nat. Commun. 2017, 8, 14630), and contain four major elements of ionizable cationic lipid, phosphatidylcholine, cholesterol, and PEG-lipid. FPLC-purified m1Ψ-containing, firefly luciferase, eGFP or control Poly(C) (Sigma) were encapsulated in LNPs at an RNA-to-total lipid ratio of ˜0.05 (wt/wt) using a self-assembly process as elaborated before (Pardi, et al., Nat. Commun. 2017, 8, 14630). mRNA-LNP formulations were kept frozen at −80° C. at a concentration of mRNA of ˜1 mg/mL.

### Preparation and Characterization of Targeted Lipid Nanoparticles

To target mRNA-loaded lipid nanoparticles to endothelial cells, LNPs were conjugated with mAb specific for PECAM and ICAM-1. Targeting antibodies or control isotype-matched IgG was conjugated to LNP particles via SATA-maleimide conjugation chemistry (Howard, et al., Mol. Pharm. 2015, 11, 2262-2270). The LNP construct was modified with maleimide functioning group (DSPE-PEG-mal) by a post-insertion technique with minor modifications (Ishida, et al., FEBS Lett. 1999, 460, 129-133). The antibody was functionalized with SATA (N-succinimidyl S-acetylthioacetate) (Sigma-Aldrich) to introduce sulfhydryl groups allowing conjugation to maleimide. SATA was deprotected using 0.5 M hydroxylamine followed by removal of the unreacted components by G-25 Sephadex Quick Spin Protein columns (Roche Applied Science, Indianapolis, Ind.). The reactive sulfydryl group on the antibody was then conjugated to maleimide moieties using thioether conjugation chemistry. Purification was carried out using Sepharose CL-4B gel filtration columns (Sigma-Aldrich). mRNA content was calculated by performing a modified Quant-iT RiboGreen RNA assay (Invitrogen).

Size and surface charge analysis of the mRNA containing lipid nanoparticles was performed using dynamic light scattering (DLS) and laser doppler velocimetry (LDV), respectively on a Malvern Zetasizer Nano ZS (Malvern Instruments, Worcestershire, UK). For size measurements, LNPs were diluted in PBS pH 7.4 and the experiment carried out at 25° C. in disposable capillary cuvettes. A non-invasive back scatter system (NIBS) with a scattering angle of 173° was used for this measurement. Diameters of unmodified and antibody-modified particles were interpreted as normalized intensity size distribution as well as z-average values for particle preparations. Zeta potential measurements were also carried out in PBS buffer using disposable folded capillary cells.

Morphology characterization was carried out on a JOEL1010 transmission electron microscope (TEM) following the protocol mentioned above (Khoshnejad, et al., Bioconjug Chem 2016, 27, 628-637). Briefly, carbon-coated 200-mesh copper grids were placed on a drop of the sample for 2 min, then washed with Milli-Q water. Negative staining was done using 2% uranyl acetate. The stain was then wicked off with a filter paper and the grids were dried and imaged at an acceleration voltage of 120K.

In Vitro Cell Binding Assay with Radiolabeled Particles

LNPs were first radiolabeled with Na¹²⁵I using Iodination Beads (Pierce). The reaction was performed for 15 min at room temperature. Unreacted materials were then removed by Quick Spin Protein Columns (G-25 Sephadex, Roche Applied Science, Indianapolis, Ind.) (Khoshnejad, et al., Bioconjug Chem 2016, 27, 628-637). Antibody conjugation was evaluated by incubation of REN-PECAM cells, which stably express PECAM, with anti-PECAM targeted LNPs. Wild-type REN cells, a human mesothelial cell line that has no endogenous expression of PECAM, was tested in parallel to assess non-specific binding of particles. To validate targeting efficiency of particles in an activated state in which ICAM-1 becomes upregulated, we tested the particles in HUVECs treated with TNF-α. Both REN cells and HUVECs were incubated with increasing quantities of LNPs for one hour at room temperature. Incubation medium was then removed and cells were washed with PBS buffer three times to remove the unbound nanoparticles from the cell surface. The cells were lysed with 1% Triton X100 in 1 N NaOH and the cell-bound radioactivity was measured by a Wallac 1470 Wizard gamma counter (Gaithersburg, Md.) and compared to total added activity.

In Vitro Cell Transfection with Reporter mRNA-Loaded LNPs

HUVECs were seeded in 48-well plates at 25,000 cells per well. After 18 hours, the cells were treated either with TNF-α (10 ng/mL) or PBS for another 5 hours. LNPs carrying reporter luciferase mRNA were added at increasing concentrations to the cells, and incubated for 1.5 hours. Plates were then washed three times with PBS and complete medium was added to the cells. After culturing for 24 hours in complete media, cells were washed with PBS, lysed in luciferase cell culture lysis reagent (Promega, Madison, Wis.) and the luciferase protein activity as luminescence (Luciferase assay system, Promega) was measured. REN cells were treated similarly, excluding the TNF-α treatment. Transfections in all cases were performed in triplicates.

For fluorescence microscopy, HUVECs were plated at 150,000 cells per well in 24-well plate. At ˜70% cell confluence, LNPs carrying eGFP mRNA were added to the media and cells incubated for 18 hours. The level of eGFP production was then evaluated by imaging the cells under an EVOS-FL imaging system (Thermofisher scientific, Waltham, Mass.).

### Local Brain Injury Model

Intrastriatal (i.s.) TNF injection was performed (Montagne, et al., 2012, Neuroimage, 63:760-770). Briefly, mice were anesthetized using an IP administration of ketamine/xylazine and placed in a rodent stereotaxic frame (David Kopf Instruments, Tujunga, Calif., USA). A total 2.5 μL of 200 μg/mL mouse-recombinant TNF (R&D systems, Minneapolis, Minn., USA) in PBS was administered by i.s. injection using a 10-0 Nanofil microsyringe over a 3-min period. Control animals did not receive any surgical procedure to avoid any induced inflammation.

### Pharmacokinetics/Biodistribution Studies Upon Intravenous Injection of Radiolabeled LNPs in Mice

Radiolabeled LNPs were administered by retro-orbital injection in normal C-57BL/6 mice (The Jackson Laboratory, Bar Harbor, Me.). The animals were sacrificed at 5, 15, 30, and 60 minutes post-injection and their blood was collected via the inferior vena cava. Organs (liver, spleen, lung, kidney, heart, and brain) were harvested, rinsed with saline, blotted dry, and weighed. Tissue radioactivity in organs and 100-μl samples of blood was measured in a gamma counter (Wallac 1470 Wizard gamma counter, Gaithersburg, Md.). Radioactivity values and weight of the samples were then used to calculate targeting parameters of nanoparticles, including tissue uptake as percent of injected dose per gram tissue (% ID/g), and localization ratio (LR) as organ-to-blood ratio. Immunospecificity index (ISI) was also calculated as the ratio of the LR of targeted particles to that of non-targeted (IgG) control. These parameters were employed to discuss biodistribution and effectiveness of antibody-targeted formulation uptake in desired tissue.

### Functional Activity—Luciferase Transfection In Vivo

Mice (The Jackson Laboratory, Bar Harbor, Me.) were intravenously injected with unmodified or antibody-modified LNP formulations. At desired time points, animals were euthanized and all the vital organs were resected, washed with PBS, and stored at −80° C. until analysis.

Organ samples were homogenized in 1 ml of cell lysis buffer (1×) (Promega Corp, Madison, Wis.) containing protease inhibitor cocktail (1×) and mixed gently at 4° C. for one hour. The homogenates were then subjected to cycles of freeze/thaw in dry ice/37 OC. The resulting cell lysate was centrifuged for 10 min at 16,000 g at 4° C. Luciferase activity was assayed in the supernatant using a Victor³ 1420 Multilabel Plate Counter (Perkin Elmer, Wellesley, Mass.).

### Bioluminescence Imaging

Bioluminescence imaging was performed as described previously (Pardi, et al. J. Control. Release 2015, 217, 345-351) using an IVIS Spectrum imaging system (Caliper Life Sciences, Waltham, Mass.). Mice were administered an intraperitoneal injection of D-luciferin at a dose of 150 mg/kg. After 5 min, the mice were euthanized; organs were quickly harvested, and placed on the imaging platform. Organ luminescence was measured on the IVIS imaging system using an exposure time of 5 s or longer to ensure that the signal obtained was within operative detection range. Bioluminescence values were also quantified by measuring photon flux (photons/second) in the region of interest using LivingImage software provided by Caliper.

### Statistical Analysis

Unless specified otherwise, the data have been calculated and presented as mean±standard error of mean (SEM). When comparing two groups, a Student's t-test was used assuming a Gaussian distribution with unequal variances. All probability values are two-sided, and values of p<0.05 were deemed statistically significant.

The results of the experiments are now described.

### Physicochemical Characterization of Targeted Lipid Nanoparticles

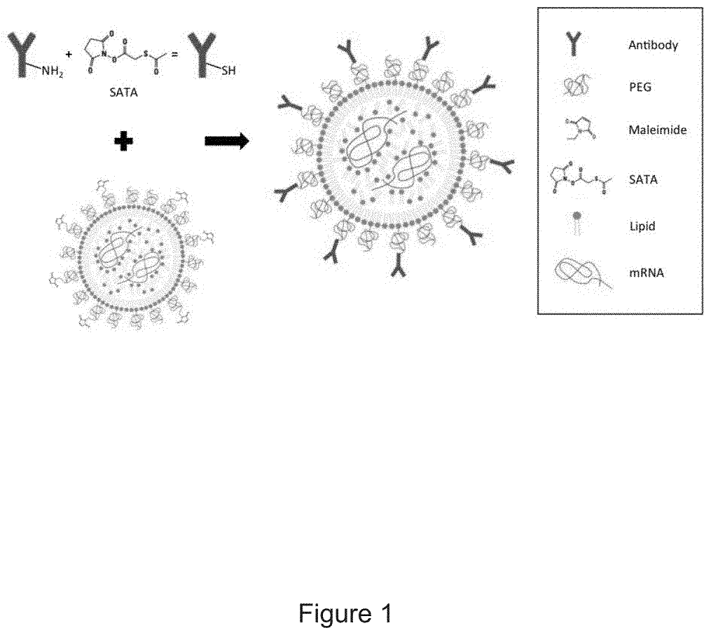

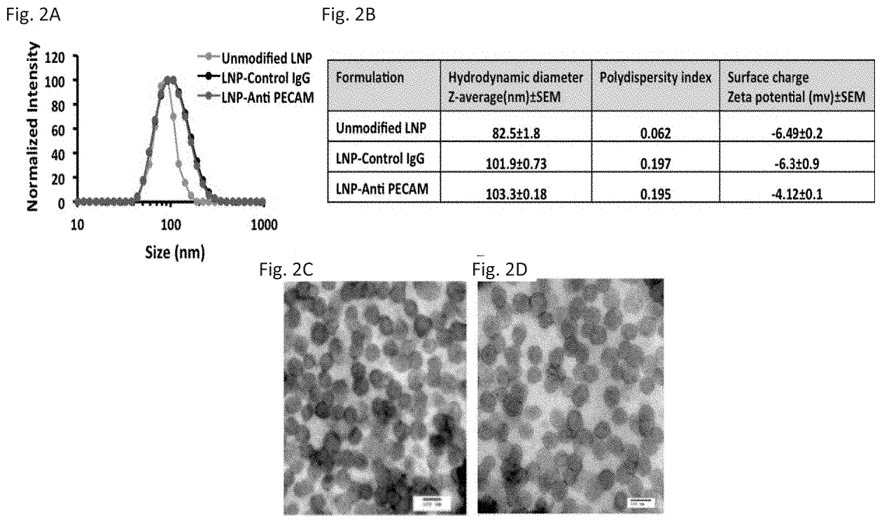

A schematic describing the conjugation of Ab to LNPs is illustrated in FIG. 1. After antibody conjugation, particle size and surface charge were used to compare the physical characteristics of antibody-conjugated LNPs to those of unmodified LNPs. Dynamic light scattering measured a hydrodynamic diameter of 82.5±1.8 nm with a very narrow size distribution (PDI=0.062) for unmodified LNP. As demonstrated in FIG. 2A and FIG. 2B, upon coupling antibody to LNPs, the mean z-average of particles increased up to ˜100 nm (101.9±0.73 nm) for LNP-control IgG and 103.3±0.18 nm for LNP-anti PECAM. This 20 nm increase in particle size can be indicative of a thin antibody layer coating on the LNP core. As expected, adding a new component to the LNP construct increased the polydispersity index of the formulation to 0.2. Antibody-conjugated formulations had a negative zeta potential of −6.3 to −4, very similar to the surface charge of unmodified formulations (−6.49±0.2). Therefore, antibody conjugation did not significantly affect the surface charge of the particles. The morphology of LNPs, prior (unmodified LNP, FIG. 2C) and following anti-PECAM conjugation (antibody-conjugated LNP, FIG. 2D), was visualized by TEM, which revealed that antibody-LNP conjugates retain the characteristic structure of lipid nanoparticles, representing a fairly homogenous population of spherical particles.

### Binding of Targeted Lipid Nanoparticles to Endothelial Cells In Vitro

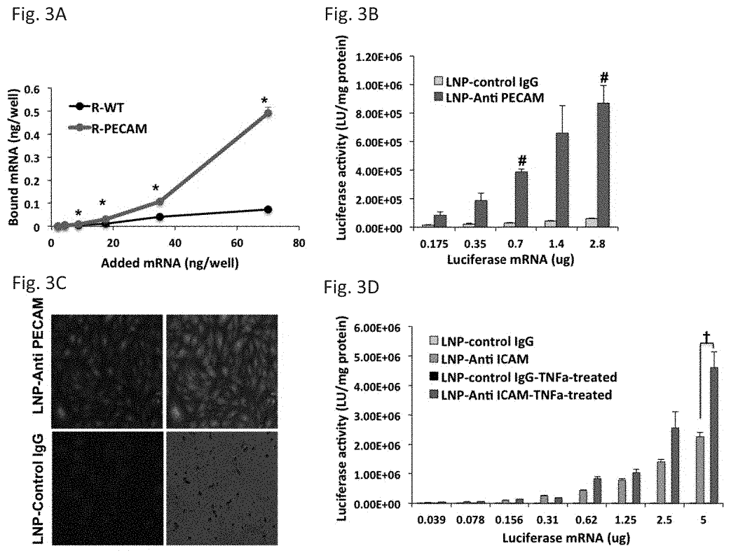

The binding capability of the targeted LNPs was first evaluated on a model endothelial cell line consisting of human mesothelioma (REN) cells transfected with human PECAM-1, REN-PECAM (Gurubhagavatula, et al., J. Clin. Invest. 1999, 101, 212-222). In this experiment, wild-type REN cells, which have no endogenous expression of PECAM-1 were used as a control cell line. As shown in FIG. 3A, using anti-PECAM targeted LNPs, a relatively high affinity binding was observed, with minor non-specific binding to REN wild type cells.

After cell binding validation, the functional activity of luciferase encoding mRNA included in targeted vs. non-targeted formulations was measured in REN-PECAM cells. The enhanced targeting ability of anti PECAM-targeted LNPs demonstrated improved functional effect (luciferase activity) compared to non-targeted LNPs (LNP-control IgG) in dose-dependent manner (FIG. 3B). There was also an increasing trend in functional activity of LNPs with increasing doses of mRNA, demonstrating a dose-response correlation. Transfection and mRNA translation of anti-PECAM targeted LNPs in REN-PECAM cells was analyzed by delivering mRNA expressing eGFP (eGFP mRNA) to the same cell line with targeted (LNP-anti PECAM) or non-targeted (LNP-control IgG) formulations. As presented in FIG. 3C, fluorescence intensity of cells received the targeted formulation is significantly higher than the ones that received the non-targeted formulation.

While REN cells are a versatile model for examining binding and specificity, their expression of stably transfected PECAM-1 gene does not essentially represent that seen on endothelial cells. To get a more physiologic measurement of in vitro binding and activity, luciferase activity was next measured in HUVECs, primary human endothelial cells, transfected with either LNP-anti ICAM or LNP-control IgG. This also enabled the testing of the targeting platform in an activated state. To do so, HUVECs were incubated with TNF-α at a concentration of 10 ng/ml in medium for 5 hours before LNP administration. TNF-α activates endothelial cells and induces higher expression of cell adhesion molecules (CAMs), such as ICAM-1. FIG. 3D reveals that targeted LNPs (in this case, LNP-anti ICAM) drastically outperformed the non-targeted formulations at all concentrations tested in both HUVEC and TNF-α-activated HUVECs. Targeted (LNP-anti ICAM) but not control IgG-coated LNPs causes luciferase expression in the endothelial cells in a dose-dependent manner (FIG. 3D). Moreover, there was a marked enhancement (almost 2-fold increase) in transfection efficiency of targeted formulations in TNF-α-treated cells versus non-treated cells particularly visible at high mRNA doses, indicating that the high level of target expression correlates with increased transfection. This shows the potential of the targeting platform to be used in pathologic conditions associated with inflammation, where upregulation of CAMs on endothelial cells occurs.

Targeting of mRNA-Loaded Lipid Nanoparticles to Vascular Endothelium In Vivo

The biodistribution of targeted particles was next analyzed in mice after intravenous administration. To measure tissue uptake, the amount of radioactivity as percent of injected dose per gram of tissue (% ID/g) was calculated. LNPs were directly labeled with ¹²⁵I prior to conjugation, therefore, measured radioactivity only showed distribution of particles without any detached targeting antibodies affecting the outcome.

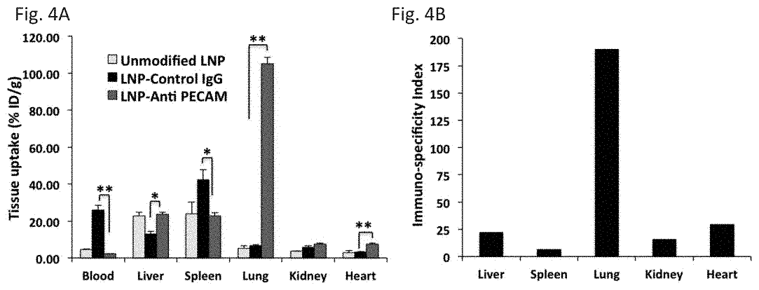

For unmodified particles, the highest accumulation was detected in the liver and spleen. In case of control IgG-coated particles (LNP-control IgG), the highest accumulation of particles occurred in the spleen (42.29±5.56% ID/g) (FIG. 4A). Importantly, a substantial amount of particles were still circulating in the blood (25.84±2.62% ID/g) at 30 minutes, representing a significant impact on biodistribution caused by the control IgG coating. On the other hand, for targeted LNPs (LNP-anti PECAM) the majority of the uptake was in the lung 105.03±3.49% ID/g, representing a 16-fold increase in pulmonary uptake vs. the non-targeted formulation. To a lower extent, targeted particles were also increased in kidney and heart (7.60±0.39 and 7.59±0.37% ID/g, respectively), compared to non-targeted counterparts (5.83±0.84 and 3.08±0.33% ID/g, respectively). However, this was negligible in comparison with the effect observed in lungs. The localization ratio (LR), defined as the ratio of % ID/g of a given organ to that in the blood, was also calculated for both PECAM-targeted and untargeted LNPs. The immunospecificity index (ISI), or the ratio of the LR of the targeted particle to that of untargeted (IgG) control, was 190 in lung tissue (FIG. 4B).

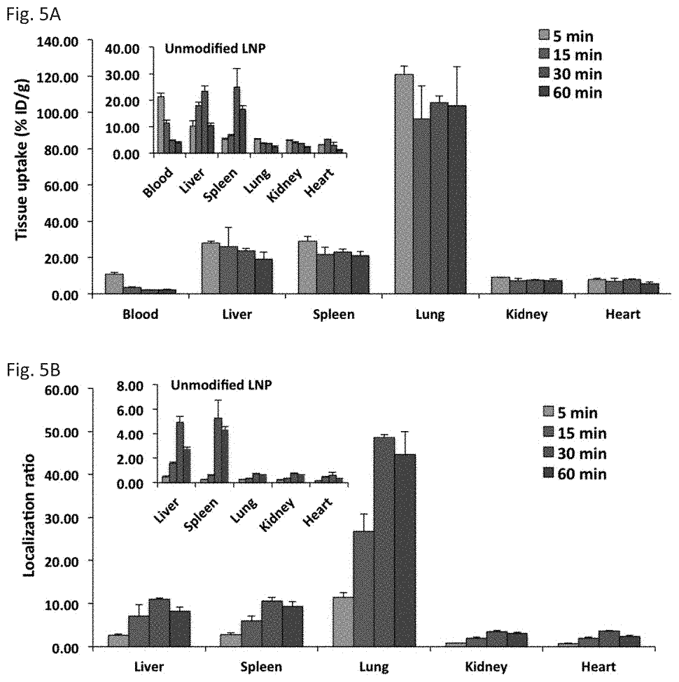

Comprehensive kinetic studies were conducted to further explore and quantitate in vivo tissue uptake kinetics of LNP formulations. ¹²⁵I-labeled LNPs were traced after IV injection in mice at 5, 15, 30, and 60 minutes. Both unmodified and PECAM-targeted LNPs were cleared from blood very quickly with the highest circulating amount of unmodified LNP and LNP-anti PECAM at 21.35±1.26 and 10.75±0.89% ID/g in blood, respectively at the earliest time point tested i.e. 5 minutes (FIG. 5A). At later time points, the concentration of particles in blood quickly dropped to a % ID/g of 3.88±0.46 for unmodified LNP and 2.29±0.18 for PECAM-targeted LNP at the latest time point, i.e. 60 minutes. Unmodified LNP accumulated mainly in liver and spleen (FIG. 5A inset) while PECAM targeted LNPs accumulated efficiently in lung (FIG. 5A and FIG. 5B). Specific lung uptake of targeted particles peaked at the first time point post-injection and the localization ratio increased over time staying stable through the last time point, 60 minutes (FIG. 5A and FIG. 5B). LNPs were bound at high concentration in lung tissue for an extended time period.

Tissue Transfection Pattern Upon Administration of mRNA-Loaded Lipid Nanoparticles Targeted to Vascular Endothelium In Vivo

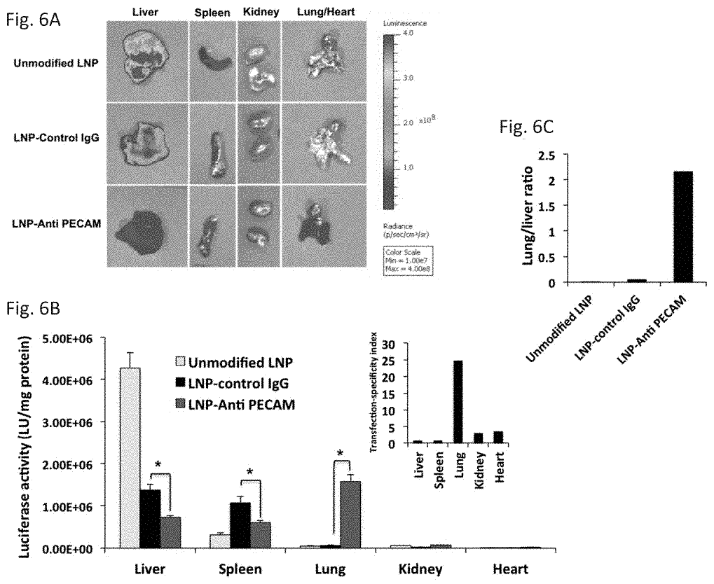

mRNA translation after IV administration of targeting particles was then analyzed. Unmodified LNP, LNP-control IgG, and LNP-anti-PECAM containing luciferase encoding mRNA were first administered at a dose of 8 μg (0.32 mg/kg) mRNA by retro-orbital injection. Protein expression with mRNA delivery is expected to peak around 4-5 hours after injection (Pardi, et al. J. Control. Release 2015, 217, 345-351). Therefore, 4.5 hours following injection, bioluminescence was used to detect the location of protein expression (FIG. 6A). For unmodified LNP, luciferase expressed mainly in liver and at lower level in spleen. Conjugating control IgG changed the transfection pattern as the luminescence signal decreased markedly in liver, however, without having a specific ligand, these particles circulate in blood for longer time rather than accumulating in other specific organs (FIG. 6A). More importantly, anti-PECAM targeted LNPs showed a profound and specific expression of luciferase in the lung. The luminescence was significantly lower in liver for targeted LNPs compared to control IgG-LNPs. To quantify the luciferase expression, the luciferase activity was measured in tissue harvests upon injection of unmodified LNP, LNP-control IgG, and LNP-anti PECAM. Selected organs were harvested and luciferase activity (LU/mg protein) in tissue extract was applied for interpretation of mRNA transfection in different tissues. As presented in FIG. 6B, luciferase activity for LNP-anti PECAM was ˜25 fold higher than LNP-control IgG in lung tissue. Lung luciferase expression for control IgG-conjugated LNPs (6.4×10⁴ LU/mg) was comparable to unmodified LNPs (4.5×10⁴ LU/mg). On the other hand, transfection efficiencies in liver and spleen were substantially lower for endothelial-targeted particles than for IgG-coated ones. Transfection specificity index was computed as the ratio of luciferase activity in mice treated with targeted vs. non-targeted LNPs. The transfection specificity index of PECAM-targeted particles was 24.7 in the lung and approximately 0.5 in liver and spleen (FIG. 6B). The lung/liver ratio was 0.01, 0.05, and 2.2 for unmodified LNP, LNP-control IgG, and LNP-anti PECAM, respectively (FIG. 6C).

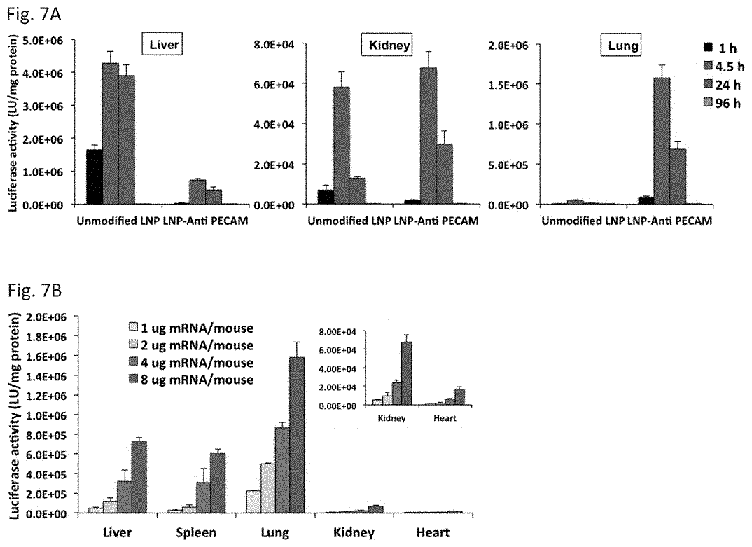

Experiments were conducted to further characterize the time course of mRNA translation after lung transfection using luciferase mRNA containing LNPs. Four time points, 1, 4.5, 24, and 96 h post-injection were chosen, based on previous studies (Pardi, et al. J. Control. Release 2015, 217, 345-351). It is notable that all three formulations, unmodified-, IgG-, and anti-PECAM-LNPs reached their maximal expression after 4.5 hours and declined slowly in the next 24 hours (FIG. 7A). At 96 hours post-injection, however, the expression value considerably diminished. It should be mentioned that at all time points tested, the endothelial targeted formulation kept its specificity to lung when compared to unmodified or non-targeted (control-IgG-LNP) formulations (FIG. 7A).

A linear dose response of IV injection of anti-PECAM-LNP containing luciferase mRNA at 4.5 hours post-injection was observed (FIG. 7B). No saturation phenomenon over the dose range evaluated (1-8 μg per mouse) was observed.

Transfection Biodistribution of Luciferase mRNA-Loaded Lipid Nanoparticles in ApoE Knockout Mice

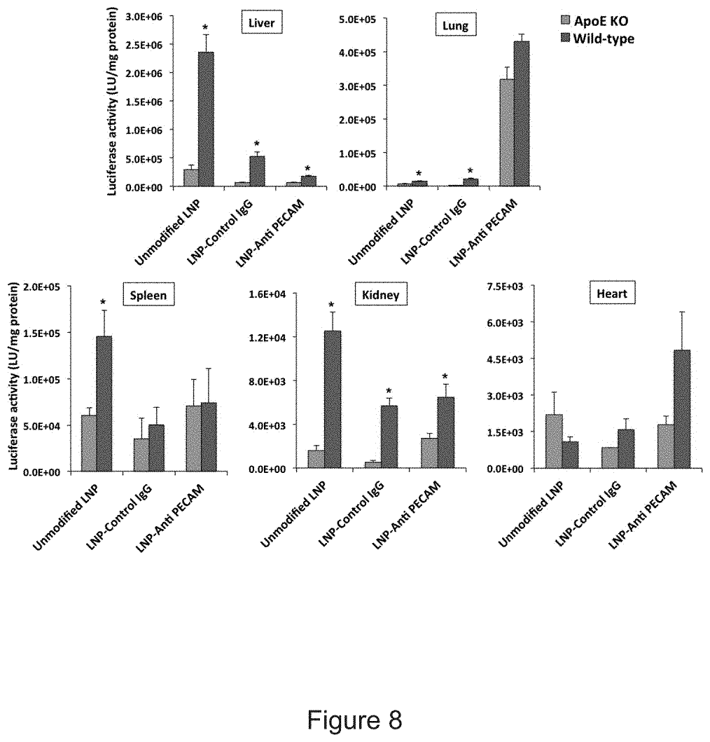

The lipid-based nanoparticles being used are known to interchange components with the serum and adsorb several types of proteins (Semple, et al., Adv. Drug Deliv. Rev. 1998, 32, 3-17). Specifically, apolipoprotein E (apoE) is adsorbed onto LNPs and enhance their uptake into hepatocytes (Akinc, et al., Mol. Ther. 2010, 18, 1357-1364). To determine whether antibody conjugation affected the liver-oriented apoE-dependent biodistribution profile of LNPs, the functional activity of luciferase mRNA containing LNPs in were compared in wildtype (WT) versus ApoE^{-/-} mice. At 4.5 hours post-IV injection of LNPs, organs were collected and luciferase expressions were measured in tissue homogenates. A marked decrease (˜8 fold) in liver transfection efficiency in ApoE^{-/-} mice was observed compared to WT mice upon receiving intact LNPs (FIG. 8). Interestingly, expression levels in other organs of ApoE^{-/-} animals was also lower than their counterparts in WT mice (FIG. 8). This suggests that similar to liver the uptake of the neutral LNPs by spleen, kidney, and to some extent by lung is also apoE-dependent (Yan, et al., Biochem. Biophys. Res. Commun. 2005, 328, 57-62).

Although a trend similar to unmodified LNP was detected for endothelial targeted LNPs, lung transfection with LNP-anti-PECAM was reduced to a much lesser extent in ApoE knockout mice. This demonstrates that the presently described effective active targeting strategy attenuates the impact of endogenous serum components like apoE from diverting the transfection to liver.

### Targeting and Activity of Targeting Platform in a Local Brain Injury Model

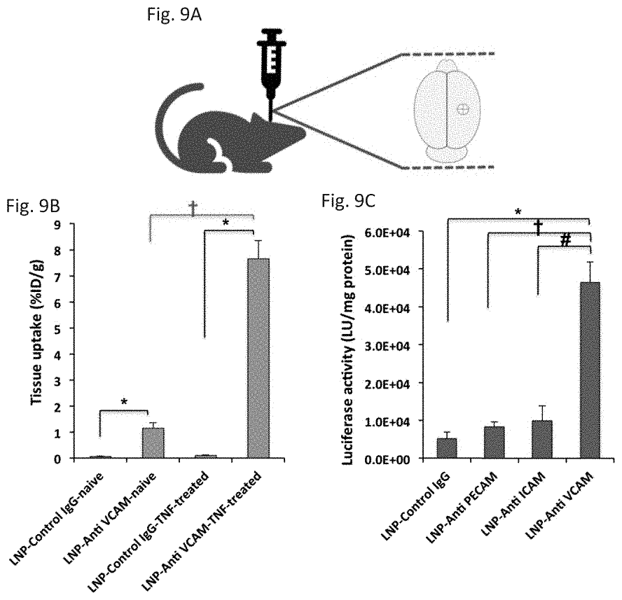

Further experiments were conducted using a physiologic model involving the brain to demonstrate the versatility of the targeting system. Cytokines, such as tumor necrosis factor alpha (TNF-α) are important mediators of inflammatory diseases in the CNS (Probert & Selmaj, J. Neuroimmunol. 1997, 72, 113-117). They induce the expression of adhesion molecules on the surface of brain endothelial cells and are associated with blood-brain barrier (BBB) disruption (Fabry, et al., Immunol. Today 1994, 15, 218-224). One of the most important adhesion molecule receptors that is highly upregulated on endothelial cells upon TNF-α stimulation is vascular cell adhesion molecule 1 (VCAM-1) (Carlos, et al., Blood 1990, 76, 965-70). Therefore, intrastriatal (i.s.) TNF administration into mouse right striatum was used to create a localized brain inflammatory model similar to a previous model (Montagne, et al., Neuroimage 2012, 63, 760-770). The IV injection of anti-VCAM-LNP elevated cellular uptake and translation in the ipsilateral hemisphere (inflamed zone) up to 71-fold compared to control-IgG-LNP (FIG. 9A). The transfection efficiency of VCAM-targeted luc mRNA-LNPs was then compared to control-IgG counterparts and other common CAM-targeted LNPs, including ICAM and PECAM. Anti-VCAM LNPs induced a marked increase in luciferase expression when compared to other CAM-targeted LNPs in the ipsilateral hemisphere model (FIG. 9C). Overall, the data generated from the studies on the brain demonstrated that the targeting platform can be effectively adapted for pathological conditions, as presented here in a brain injury model.

Messenger RNA (mRNA) with its transient action has created a huge optimism for employment of mRNA-based biotherapeutics especially in severe acute conditions (Weissman, D. & Karikó, K. Mol. Ther. 23, 1416-1417; Pardi, et al., Nature 2017, 543, 248-251; Kaczmarek, et al., Genome Med. 2017, 9, 60; Kariko, Mol. Ther. 2012, 20, 948-953). The presently described studies demonstrate the development of mRNA containing LNPs targeted to the lungs by direct covalent immobilization of Ab against endothelial determinants, PECAM-1 and ICAM-1. Platelet-endothelial adhesion cell molecule-1, CD31 (PECAM-1) is mainly expressed by the endothelium and is mostly localized on the endothelial intercellular junctions (Scherpereel, et al., J. Pharmacol. Exp. Ther. 2002, 300, 777-786). Intercellular adhesion molecule-1, CD54 (ICAM-1) is also constitutively expressed on the apical endothelial plasmalemma, and gets upregulated upon inflammation (Khoshnejad, et al., Bioconjug Chem 2016, 27, 628-637). A biodegradable polymer-lipid hybrid nanoparticle formulation based on poly(β-amino esters) and lipid-polyethylene glycol (PEG) was also described by Anderson group (Kaczmarek, et al., Angew Chem Int Ed Engl 2016, 55, 13808-13812) for systemic administration of mRNA to the lungs. However, these and other similar studies on current formulations for mRNA delivery are mainly focused on inherent nature of nanoparticle materials to target endothelium rather than specifically designed targeting. Enabling specific, ligand-mediated endothelial targeting of LNP-mRNA carriers is the principal novelty of the present study.

In present work, targeting nanocarriers were able to specifically bind to and transfect the model endothelial cells line expressing PECAM-1 (REN-PECAM cells). The expression response linearly correlated to the received dose. Using HUVECs as a more physiologic representative of endothelial cells, it was shown herein that ICAM-1 targeted LNPs could result in both specific binding and transfection activity. In accordance with previous studies (Khoshnejad, et al., Bioconjug Chem 2016, 27, 628-637; Muro, et al. J Pharmacol Exp Ther 2006, 317, 1161-1169; Bhowmick, et al., J. Control. Release 2012, 157, 485-492), ICAM-1 targeted nanoparticles demonstrated higher specificity and functional activity in TNF-α treated HUVECs that upregulate ICAM-1. The extraordinary specificity of ICAM-targeted LNPs in transfecting TNF-α treated cells depict the high capacity of this LNP-mRNA targeting platform to be used in pathologic conditions, namely inflammatory states where CAM levels are upregulated on endothelial cells.

In in vivo studies, anti-PECAM targeted LNPs largely accumulated in the lung. It was also observed that superior expression in the preferred location achieved by affinity carriers was complemented by a marked reduction of hepatic expression. As observed here, targeting inhibited hepatic localization by PECAM-targeted LNPs by 47% compared to control-IgG and by 83% compared to unmodified LNPs (FIG. 5). This demonstrates that the targeting strategy redirected resulting protein expression from liver to lung. For unmodified LNP-mRNA protein expression occurred predominately in the liver, consistent with previous reports (Pardi, et al. J. Control. Release 2015, 217, 345-351; Maier, et al., Mol. Ther. 2013, 21, 1570-8). Targeting to PECAM effectively resulted in locating LNPs in the lung for extended times required for sufficient cell uptake and transfection. An interesting point is that although transfection patterns correlate with tissue uptake distributions, it does not appear that the expression capacity of pulmonary cells is comparable with that of hepatic cells. For instance, a localization ratio of anti-PECAM targeted LNPs in lung reached a value of 50 at 30 minutes after administration, however, the same parameter for unmodified LNP in liver at the same time point had at much lower value of 5. In other words, PECAM-targeted particles are taken up much more efficiently in lung compared to the level unmodified LNPs are taken up by liver. In contrast, luciferase expression in liver upon unmodified LNP administration is almost 3 times higher than the one achieved in lung after LNP-anti-PECAM administration. This comparison shows the higher capacity of hepatic cells for mRNA translation than pulmonary cells even if they do not take up extremely high number of particles. This reveals the importance of specifically targeted formulations to localize mRNA-LNPs at high concentration in desired site, such that they can induce enough expression even if the desired cells are not proficient at high exogenous mRNA translation. The currently described targeting platform also demonstrates a suitable dose-response for dose tuning in therapeutic applications. The expression kinetics of mRNA was demonstrated in previous studies as having a rapid kinetic profile (Pardi, et al. J. Control. Release 2015, 217, 345-351; Turnbull, et al., Mol. Ther. 2015, 24, 1-10), since translation occurs in the cytosol without having to cross the nuclear membrane, leading to a transient expression profile. In the current study, we first confirmed the fast expression kinetics by comprehensive luciferase assays at time points ranging from 4.5 hours to 4 days post-delivery. Using luciferase mRNA, where luciferase activity is rapidly lost (Thompson, et al., Gene 1991, 103, 171-177) as a model cargo in LNP formulations, at both targeted and non-targeted stages, expression level was negligible in tissues at 96 h post-injection. Although hypothesized previously (Khan, et al., Angew Chem Int Ed Engl 2014, 53, 14397-14401), the instant results demonstrate for the first time in ApoE^{-/-} mice that affinity moieties like anti-PECAM could maintain targeting to desired tissues, lung, in the absence of apoE. This further highlights the significance of targeting moieties in mitigating the influence of endogenous serum components like apoE from averting the transfection from desired tissue to liver.

To validate the efficacy of the targeting platform for mRNA delivery to other tissues and especially in diseased states, the LNP-mRNAs were examined in a TNF-induced unilateral brain injury model. Although intra-arterial (i.a.) reperfusion therapy has attracted broad attention and entered into clinical trials especially for stroke patients (Goyal, et al., N. Engl. J. Med. 2015, 372, 1019-30; Campbell, et al., N. Engl. J. Med. 2015, 372, 1009-1018), recanalization is often ineffective with severe adverse events. On the other hand, IV administration is always seen as a feasible route for therapeutics administration among a variety of diseases. VCAM-1 was chosen as the target for mRNA delivery to cerebrovascular endothelium, since it is mainly expressed in association with inflammation (Walczak, et al., Stroke 2008, 39, 1569-1574; Brea, et al., Cerebrovasc. Dis. 2009, 27, 48-64), and has been used as an effective target for drug delivery in numerous preclinical studies (Gorelik, et al., Radiology 2012, 265, 175-185; Hoyte, et al., J. Cereb. Blood Flow Metab. 2010, 30, 1178-1187). The results presented here indicate the specific uptake of VCAM-targeted mRNA-LNPs. This is the first demonstration that a cerebrovascular-targeted delivery platform could obtain specific tissue uptake and transfection in brain of mRNA cargo. This study sheds light on the development of targeted delivery systems for making mRNA therapeutics available for pathological CNS conditions including stroke, TBI, and MS.

Reported herein is a lung-targeting, mRNA containing LNP system which generates highly localized protein expression in the lung with minimal off-target effect. The targeted platform was designed onto one of the most efficient LNP-mRNAs in systems developed by far. The present results provide the first demonstration of systemic mRNA delivery to the lung using targeted LNPs decorated with affinity moieties against vascular endothelium. Using ligands capable of attaching to molecules on the endothelial surface allows for designing of diverse targeted endothelial LNP-mRNA therapeutics. Specific rapid and transient expression of luciferase mRNA was measurable at a time window of 4-24 hours, with limited off-target biodistribution. Importantly, these data exhibit a correlation between dosing and in vivo efficacy. The current study also provides new insight in apoE-dependent uptake of LNPs. The efficacy of targeted formulations in lung tissue of WT mice was comparable to that in ApoE−/− mice. While not wishing to be bound to any particular theory, this is presumably due to the lower chance of ApoE molecules to get adsorbed onto the targeted nanoparticles, which already contain a layer of attached antibodies. Furthermore, the efficacy of the platform in both cell uptake and expression induction was demonstrated by targeting LNPs to inflamed brain tissue using anti-VCAM-targeted particles. This is the first time that LNPs carrying mRNA cargo was effectively shown to target and transfect cerebrovasculature tissue. In summary, this study not only highlights anti-PECAM targeted LNP-mRNA as a delivery vector to induce protein expression in lung, but also demonstrated the versatility of the targeting system to efficiently target and transfect other tissues such as brain. By changing the antibody, this methodology can be easily extended to prepare LNP-mRNAs aimed at other targets (i.e., cells types, tissue, organs).

### Example 2: Selective Targeting of Nanomedicine to Inflamed Cerebral Vasculature

Drug targeting to sites of brain pathology remains an elusive goal. Using a mouse model of local TNFα-induced acute brain inflammation, it is demonstrated herein that uptake in the inflamed brain of intravenously injected antibody to Vascular Cell Adhesion Molecule 1 (anti-VCAM) is more than 10-fold greater that of antibodies to Transferrin Receptor-1 and Intercellular Adhesion Molecule 1 (TfR-1 and ICAM-1). Likewise, uptake of anti-VCAM/liposomes exceeded that of anti-TfR and anti-ICAM counterparts by ˜27 and ˜8 fold, respectively, with a brain/blood ratio >300 times that of IgG/liposomes. Radioisotope-labeled anti-VCAM/liposomes enabled molecular imaging of acute brain inflammation in mice by SPECT/CT. Both intravital microscopy via cranial window and flow cytometric analysis of brain tissue demonstrated binding of anti-VCAM/liposomes primarily to cerebrovascular endothelial cells, and not to leukocytes infiltrating the inflamed brain. Likewise, anti-VCAM/Lipid nanoparticles (LNPs) bearing mRNA selectively localized to the brain and induced de novo expression of reporter protein. To test therapeutic effect, experiments were conducted with anti-VCAM/LNPs bearing mRNA encoding thrombomodulin (TM), an endogenous endothelial surface glycoprotein and critical regulator of coagulation, inflammation, and vascular barrier function. Anti-VCAM/LNP loaded with TM mRNA induced TM expression and alleviated TNF-α-induced cerebral edema. These results establish the utility of VCAM-1-targeting of nanocarriers for both molecular imaging of brain inflammation and endothelial drug delivery in areas of cerebrovascular activation or injury.

Vascular drug delivery to sites of cerebral pathology is an enormously important, yet challenging biomedical goal. Several strategies and their combinations are currently being explored to achieve this elusive goal. Nanomedicine uses drug carriers, both synthetic (such as liposomes) and natural (such as cells or their fragments or biomolecules—some of which might fortuitously have natural tropism to target sites) for this purpose. Auxiliary maneuvers, such as focused ultrasound, radiation, or osmotic shift, might facilitate drug uptake in the target tissue, although these come with a risk of unintended effects (Medina et al., 2007, British Journal of Pharmacology, 150:552-558).

Coupling drugs and carriers to molecules with affinity to desirable target sites in the brain holds promise to facilitate delivery of pharmacological agents to these targets. In theory, drug targeting to desirable site of action will improve therapy and, with the advent of personalized medicine, enable interventions meeting the individual needs of a patient. The immense spatiotemporal diversity of pathological processes in the brain highlights the compelling need for alternatives to the most frequently explored targets, such as the transferrin receptor (TfR) (Johnsen et al., 2016, Journal of Controlled Release, 222:32-46), insulin receptor (Boado et al., 2011, J. Drug Deliv., 1-12), P-selectin (CD62P) (Fournier et al., 2017, Proc. Natl. Acad. Sci., 114:6116-6121), and the intracellular adhesion molecule-1 (ICAM-1, or CD54) (Hsu et al., Pharm. Res., 31:1855-1866).

The Vascular Cell Adhesion Molecule-1 (VCAM-1, or CD106) is selectively upregulated and exposed on the luminal surface of endothelial cells at diverse sites of acute and chronic inflammation or injury. The human pathological conditions associated with VCAM-1 expression in activated endothelium include; atherosclerosis (Nahrendorf et al., 2012, Circulation Research, 110:902-903), rheumatoid arthritis (Leung, K., 2004, Polyethylene glycol-coated gold nanoshells conjugated with anti-VCAM-1 antibody. In Molecular Imaging and Contrast Agent Database (MICAD)), inflammatory bowel disease (Tlaxca et al., 2013, J. Control Release, 165:216-225), diabetes mellitus, ischemia/reperfusion injury, glomerulonephritis (Kuldo et al., 2013, J. Control Release, 166:57-65), sepsis (Belliere et al., 2015, Theranostics, 5) and neoplasm (Bank et al., 1993, Br. J. Cancer, 68:122-124). In the pathologies of the central nervous system (CNS), VCAM-1 has been found using immunostaining, western blotting, fluorescence-activated cell sorting (FACS), and polymerase chain reaction (PCR) in the cerebral vessels in both patients and animals with models of neurodegenerative diseases (Montagne et al., 2012, Neuroimage 63:760-770), ischemic and hemorrhagic stroke (Gauberti et al., 2013, Stroke, 44), encephalitis (Irani et al., 1996, J. Immunol., 156:3850-7) and meningitis (Polfliet et al., 2001, J. Immunol., 167:4644-4650).

Antibodies against VCAM-1 labeled with imaging probes (isotopes, quantum dots, nanoparticles and fluorescent agents) have been injected locally and systemically in animal models of many of these conditions, enabling detection of pathologic lesions and, in some cases, subclinical disease by modalities including positron emission tomography (PET), single-photon emission computed tomography (SPECT), and magnetic resonance imaging (MRI) (Nahrendorf et al., 2009, JACC Cardiovasc. Imaging, 2:1213-1222; Patel et al., 2015, Bioconjug. Chem., 26:1542-1549; Frechou et al., 2013, Contrast Media Mol. Imaging, 8:157-164). Recently, anti-VCAM/nanoparticles were used for MRI imaging of pathological changes in the brain of animals with models of ischemic stroke and acute cerebral inflammation (Gauberti et al., 2013, Stroke, 44; Orndorff et al., 2014, Am. J. Physiol.—Lung Cell. Mol. Physiol., 306; Serres et al., 2011, FASEB J., 25:4415-22).

The exquisite selectivity of VCAM-1 expression for abnormal endothelium is a particularly desirable feature for imaging applications. In drug delivery applications, however, the priority is accumulation in the target tissue of a therapeutically effective percentage of the administered dose. From this standpoint, drug targeting to VCAM-1 may seem somewhat disadvantageous in comparison with targeting to ICAM-1 and other vascular determinants that are constitutively expressed by endothelial cells. While these markers may not be as selective as VCAM-1 for areas of pathology, their several orders of magnitude higher surface density ultimately enables superior delivery to the therapeutic sites (Henninger et al., 1997, J. Immunol., 158:1825-1832). Outside the CNS, VCAM-1 has also been explored as a target for drug delivery to kidneys (Kuldo et al., 2013, J. Control Release, 166:57-65), lungs (Roblek et al., 2015, J. Control. Release, 220:341-347), atherosclerotic plaques (Kheirolomoom et al., 2015, ACS Nano, 9:8885-8897), and tumors (Gosk et al., 2008, Biochim. Biophys. Acta—Biomembr., 1778:854-863). In most instances, these studies have not included quantitative assessment of uptake into the target organ or pathologic lesion, and instead delivery has been inferred based on modification of disease phenotype or outcome. The validity of this inference is not necessarily clear, however, as a number of studies have documented poor correlation between extent of local accumulation and the therapeutic effects of targeted nanocarriers (Li et al., 2018, PLoS One, 13; Leus et al., 2014, Int. J. Pharm., 469:121-131).

The present experiments provide systematic quantitative analysis of the biodistribution parameters of candidate antibodies and range of delivery systems, both at baseline and in a mouse model of tumor necrosis factor alpha (TNFα) microinjection in the striatum. It is found that VCAM-1 provides uniquely advantageous targeting to inflamed cerebral endothelium. The selectivity and unexpected efficacy of uptake in the inflamed brain of VCAM-1 antibodies and antibody coated liposomes and lipid nanoparticles (LNP) exceed those of TfR-1 and ICAM-1 by orders of magnitude. Anti-VCAM/liposomes and anti-VCAM/LNP selectively deliver their cargo to inflamed brain, enabling, respectively, molecular imaging of the pathology and expression of mRNA encoding transgene proteins. Intravenous injection of anti-VCAM/LNP carrying mRNA for thrombomodulin (TM) provided tangible alleviation of cerebrovascular edema induced by TNFα. These results identify and validate VCAM-1 targeting as a novel approach for therapeutic interventions in the brain.

The materials and methods employed in these experiments are now described.

Reagents and Hybridoma Cell Lines

Reagents for preparation of liposomes were purchased from Avanti Polar Lipids (Alabaster, Ala.): cholesterol, DPPC (1,2-dipalmitoyl-sn-glycero-3-phosphocholine), DSPE-PEG(2000) Azide (1,2-distearoyl-sn-glycero-3-phosphoethanolamine-N-[azido(polyethylene glycol)-2000], DTPA-PE (1,2-distearoyl-sn-glycero-3-phosphoethanolamine-N-diethylenetriaminepentaacetic acid), and TopFluor TMR PC (1-oleoyl-2-(6-((4,4-difluoro-1,3-dimethyl-5-(4-methoxyphenyl)-4-bora-3a,4a-diaza-s-indacene-2-propionyl)amino)hexanoyl)-sn-glycero-3-phosphocholine). DBCO-PEG4-NHS ester was purchased from Jena Bioscience (Jena, Germany). Recombinant TNFα was from PreproTech (Rocky Hill, N.J., USA) and BioLegend (San Diego, Calif.), who also supplied PerCP/Cy5.5-conjugated anti-CD45 antibody (clone 30-F11). Rat IgG control antibody was purchased from ThermoFisher Scientific (Nashville, Tenn.), anti-mouse CD71 antibody (clone RI7217.1.4) was purchased from eBioscience (San Diego, Calif.), monoclonal ANTI-FLAG M2-Peroxidase (HRP) antibody purchased from Sigma (St. Louis, Mo.), mouse Thrombomodulin/BDCA-3 antibody, goat anti-mouse purchased from RnD systems (Minneapolis, Minn.) and donkey anti-goat IgG-HRP from Santa Cruz (Dallas, Tex.). All additional antibodies—anti-mouse-VCAM-1 (clone MK2.7), anti-mouse-ICAM-1 (clone YN1/1.7.4), and anti-mouse-PECAM-1 (clone 390)—were produced by culturing hybridoma cells, purified using protein G sepharose (GE Healthcare Bio-Sciences, Pittsburgh, Pa.) and dialyzed in PBS.

Stable Cell Lines

REN cells, a human mesothelioma cell line, and REN-mICAM-1 cells, which stably express mouse ICAM-1 on their surface, have been described previously (Greineder et al., 2013, PLoS One, 8). To create REN cells stably expressing mouse VCAM-1, a full-length cDNA for mouse VCAM-1 was purchased from GenScript (Piscataway, N.J.). The 2232 bp cDNA was cloned into the pcDNA 3.1(+) vector between HindIII and BamHI restriction enzyme sites and transfected into REN cells using Lipofectamine 2000 (Life Technologies, Grand Island, N.Y.). Stably expressing cells were selected in media containing 200ug/mL of Geneticin (Life Technologies, Grand Island, N.Y.).

Modification of Antibodies

For attachment to immunoliposomes, antibodies were modified using DBCO-PEG4-NHS ester (Jena Bioscience, Jena, Germany) per manufacturer protocol. Briefly, antibodies were diluted to approximately 3 mg/mL solution in PBS and mixed at 1:15 ratio with NHS ester in DMSO, keeping the concentration of DMSO <1% v/v. After reaction for 1 hour at room temperature (RT), modified mAbs were purified from residual DBCO reagent using Amicon Ultracel-50 kDa membrane filter (Millipore, Burlington, Mass.). For fluorescent labeling, antibodies were modified using AlexaFluor-488 or AlexaFluor-647 NHS Ester reagents (ThermoFisher Scientific) and a similar protocol was followed for conjugation. Fluorescently labeled antibodies were purified via centrifugation through a 100 kDa amicon filter (Millipore).

Since radiolabelling can ablate antigen binding to varying degrees, the immunoreactivity (IR) of each labeled mAb—i.e., the fraction capable of binding target antigen—was tested prior to injection. Only radiolabeled antibodies with IR >75% were used in radiotracing experiments (the assay has a maximum of approximately 80-85%).

Preparation of Immunoliposomes

The immunoliposomes used in these experiments were formulated to allow: i) highly efficient, biorthogonal “click” chemistry conjugation for surface attachment of cyclooctyne-modified antibodies to azide functionalized phospholipids and ii) direct radiolabeling of the carrier (as opposed to targeting antibody) via chelation of ¹¹¹In onto DTPA-functionalized lipid.

Azide functionalized liposomes were prepared by thin film hydration techniques similar to those previously described (Hood et al., 2012, J. Control. Release, 163:161-9). Briefly, chloroform solutions of DSPE-PEG(2000) Azide, cholesterol, and DPPC were combined in a borosilicate glass tube at a total lipid concentration of 20 mM and phospholipid to cholesterol ratio of 3:1. Dried lipid films were hydrated and extruded ten times through 0.2 μm polycarbonate filters (Avanti Polar Lipids). Dynamic light scattering measurements of hydrodynamic particle size, distribution, and PDI were made following extrusion and at each subsequent step of modification using a Zetasizer Nano ZSP (Malvern Panalytical, Malvern UK).

For conjugation of targeting ligands, azide functionalized liposomes were incubated overnight with DBCO-modified antibodies at 37° C. Immunoliposomes were purified from residual antibody using a 20 mL Sepharose 4B-Cl column (GE Healthcare, Pittsburgh, Pa.). Fluorescent labeling of immunoliposomes was accomplished by one of two methods. For flow cytometry experiments, azide liposomes were conjugated to DBCO- and AlexaFluor-647-modified antibodies and purified as above. For intravital microscopy experiments, the fluorescent lipid, TopFluor TMR PC, was incorporated into the lipid formulation at a molar ratio of 0.5%.

Radiolabeling of Antibodies and Immunoliposomes:

Antibodies were directly radioiodinated with [¹²⁵I]Na (Perkin Elmer, Waltham, Mass.) using Pierce Iodogen radiolabeling reagent and purified using Zeba desalting spin columns (ThermoFisher Scientific). Radiochemical purity, assessed via TLC using a mobile phase of 75% methanol:25% NH₄ acetate, was >90% in all cases. To allow radiolabeling of Immunoliposomes, a chelator-containing lipid (DTPA-PE) was incorporated into the lipid mixture at a molar ratio of 0.25%. Thin films were hydrated using metal-free citrate buffer and labeled with ¹¹¹In (Nuclear Diagnostic Products, Rockaway, N.J.) for 1 hour at 37° C. Free indium was removed using an Amicon Ultracel-50 kDa membrane filter (Millipore, Burlington, Mass.). Radiochemical purity, assessed via TLC using a mobile phase of 10 mM Na-EDTA (ThermoFisher Scientific), was >95% in all cases.

Immunoreactivity Assay

The immunoreactivity of radiolabeled antibodies or liposomes—i.e., the fraction capable of binding target antigen—was determined using a simplified version of the technique originally described by Lindmo et al (Bonavia et al., 2006, Appl. Radiat. Isot., 64:470-474). Briefly, radiolabeled antibodies or immunoliposomes were incubated with aliquots of fixed cells expressing their target ligand. Small amounts of radiolabeled material were used (typically <10ng mAb or <10¹⁰ liposomes) in order to maintain at least a 30-fold excess of binding sites. REN-VCAM-1 and REN-ICAM-1 cells were used for VCAM-1 and ICAM-1 targeted species, respectively, with wild type REN cells as a control for non-specific binding. For TfR-1, mouse fixed reticulocytes were used (Newman et al., 1982, Trends Biochem. Sci., 7). Since a non-expressing control cell was not available in this case, the binding of TfR-1-targeted mAb or liposomes was compared to that of IgG controls. After 1 hour of binding at room temperature, cells were washed twice with cold PBS and pelleted by centrifugation. The immunoreactivity was defined as the percentage of radioactivity remaining in the cell pellet relative to total recovered radioactivity.

Animals

Animal studies were carried out in accordance with the Guide for the Care and Use of Laboratory Animals [National Institutes of Health, Bethesda, Md., USA (NIH)]. Male C57BL/6 mice, 6-8 week old, weighing 20-30 g (The Jackson Laboratory, Bar Harbor, Me., USA), were used for all experiments.

Statistical Analysis

Statistical tests (unless specified, one way ANOVA and Bonferroni post-hoc was applied) were performed using GraphPad Prism 5.

Intrastriatal TNFα Model

A unilateral intrastriatal injection of TNFα (0.5 μg) performed after placing the anesthetized mice in a stereotaxic frame (coordinates: 0.5 mm anterior, 2.0 mm lateral, −3 mm ventral to the bregma) (Montagne et al., 2012, Neuroimage, 63:760-770). Control animals did not undergo any surgical procedures. Injured animals were injected into the right brain hemisphere with TNFα.

Radiotracing and Autoradiography of Antibodies and Immunoliposomes

For radiotracing experiments, mice were injected intravenously with 100 μL of radiolabeled antibody, immunoliposomes or LNPs (targeted or untargeted) at selected times after receiving intrastriatal TNFα. All animals were exsanguinated 30 minutes after injection and residual blood was flushed from the brain and other tissues via transcardial perfusion of ice-cold PBS (20 mL). Tissue biodistribution of injected materials was determined by measuring the radioactivity in the blood and other tissues using a Wizard 2470 gamma counter. In some animals, cerebral autoradiography was performed by exposing a phosphor plate to 2 mm brain slices and imaging using a Typhoon 9400 molecular imager (Amersham, Piscataway, N.J.).

Brain Edema

To measure brain edema, injured animals were injected with radiolabeled albumin 21 hours after TNFα, which was allowed to circulate for 4 hours. The ratio between extravasated radiolabeled albumin and radiolabeled albumin within the bloodstream was calculated following transcardial perfusion (cpm/g brain: cpm/g blood). All studies of the vascular leakage described were performed in a blinded manner. For treatment purposes, LNPs or PBS as a control were injected 17 hours after injury, 4 hours later the radiolabelled albumin was injected and allowed to circulate for 4 more hours. Experiments were performed in blinded fashion to reduce bias. The percentage of the effect was measured considering as a 100% leakage as the value calculated for the ipsilateral hemisphere in PBS treated animals and 0% leakage as the value calculated for the ipsilateral hemisphere of the naive animals.

Brain Disaggregation, Flow Cytometry and Western Blot

To create a suspension of single cells, brains were disaggregated as previously described (Posel et al., 2016, J. Vis. Exp., doi:10.3791/53658). Briefly, organs were first placed in a 3 ml syringe with ice cold HBSS and repeatedly pushed through 18 gauge and then 21-gauge needles. Homogenate was filtered through a 100 μm nylon strainer, centrifuged, and resuspended in a 2.5 U/mL solution of dispase (ThermoFisher Scientific) and digested for 1 hour at 37° C. Following digestion, cell suspensions were passed through a 70 μm nylon strainer and treated with DNase I (600 units/ml; grade II, Sigma Aldrich, St Louis, Mo.) prior to centrifugation and demyelinization using a standard isotonic percoll (SIP) gradient. RBCs were lysed using ACK lysis buffer (Quality Biological, Gaithersburg, Md.) and cells were stained for PECAM-1 and CD45 for 30 minutes. Flow cytometry was performing using an Accuri C6 instrument (Becton Dickinson, San Jose, Calif.).

Brains were added to 1 ml of PBS supplemented with 1% protease inhibitor cocktail (Sigma P8349) and homogenized during 6 minutes at 30 Hz with 5 mm stainless steel bead using TissueLyser II (both are from Qiagen, Valencia, Calif.). Tissue homogenate was further lysed by addition of 0.25% Sodium deoxycholate, 0.25% SDS, 1 mM EDTA, 50 mM Tris-HCl, 150 mM NaCl (final concentrations). Following incubation on rotating platform for 1 hour at +4° C., homogenates were passed through an 18G needle 10 times, vortexed 5 times during 30 minutes on ice and centrifuged for 10 minutes at 16,000 g. Whole lysate (supernatant) was collected and protein concentration in the samples was measured by the DC Protein Assay (Bio-Rad, Hercules, Calif.). Samples were subjected on Ready gel 4-15% Tris-HCl (Bio-Rad). TM, FLAG and actin expressions were analyzed by Western blot using appropriate antibodies.

Targeted Lipid Nanoparticles Containing mRNA

LNPs containing either luciferase or TM nucleoside-modified mRNA were prepared, as described (Pardi et al., 2018, Journal of Experimental Medicine, doi: 10.1084/jem.20171450, Pardi et al., 2013, In Vitro Transcription of Long RNA Containing Modified Nucleosides. In: Rabinovich P. (eds) Synthetic Messenger RNA and Cell Metabolism Modulation. Methods in Molecular Biology (Methods and Protocols), vol 969. Humana Press, Totowa, N.J.; Kariko et al., 2012, Mol Ther, 20: 948-953) and conjugated with anti-VCAM-1 or control IgG. Briefly, LNP carriers were modified with DSPE-PEG-maleimide via a post-insertion technique while the antibody was functionalized with SATA (N-succinimidyl S-acetylthioacetate). SATA deprotection was carried out with 0.5 M hydroxylamine and the unreacted components were removed by G-25 Sephadex Quick Spin Protein columns (Roche Applied Science, Indianapolis, Ind.). Antibodies were then conjugated to LNP particles via SATA-maleimide conjugation chemistry (Howard et al., 2014, Mol. Pharm., 11:2262-2270). Purification was performed on Sepharose CL-4B columns (Sigma-Aldrich).

Biodistribution and Luciferase Transfection of Targeted LNPs In Vivo

Radiolabeled or non-radiolabeled LNP-luciferase mRNAs were injected intravenously in mice and the animals were sacrificed at specified timepoints post-injection. Blood was collected and selected organs were harvested. Tissue radioactivity was measured in a gamma counter (Wallac 1470 Wizard gamma counter, Gaithersburg, Md.) and tissue uptake was presented as percent-injected dose normalized to the mass of tissue.

To quantify protein expression, organs from the mice treated with non-radiolabeled LNP-luciferase mRNA were homogenized using a tissue homogenizer. Luciferase activity was then assayed in the cell lysate made from tissue homogenate on a Victor3 1420 Multilabel Plate Counter (Perkin Elmer, Wellesley, Mass.) and presented as luminescence units normalized to the mass of tissue.

TNFα Model and Intravital Microscopy

Intravital video microscopy of the brain microvasculature in the setting of acute neuroinflammation was performed as previously described (Rom et al., 2016, J. Neuroinflammation, 13:254). Briefly a cranial window of 4 mm diameter was opened and sealed with a 5 mm glass coverslip, following removal of the meninges. A cannula (“craniula”) (PlasticsOne, Roanoke, Va., USA) was placed into the subarachnoid space adjacent to the window. Intravital imaging was performed using a Stereo Discovery V20 epiflourescence microscope (Carl Zeiss AG, Oberkochen, Germany) equipped with an AxioCAM-1 MR digital camera.

Four to five days after cranial window and catheter implantation, animals received an intravenous injection of 50 μL of TopFluor PC-containing green fluorescent immunoliposomes. Baseline images (i.e. pre-TNFα) of liposome distribution were taken 30 minutes, 2 hours, and 4 hours after injection. Twenty-four hours later, 0.5 μg of TNFα was injected via the crainula, followed 2 hours later by a second intravenous injection of fluorescent immunoliposomes. Images were again obtained 30 minutes, 2 hours, and 4 hours post-liposomal injection. At each time point, 50 μL of a 0.05% (v/v) solution of rhodamine 6G (Sigma-Aldrich) was injected to enable simultaneous visualization of leukocytes and platelets. All images were analyzed using AxioVision 4.8 software and ImageJ.

SPECT/CT Imaging

For molecular imaging, mice were injected with approximately 200 μCi of ¹¹¹In-labeled liposomes approximately 16 hours after intrastriatal TNFα. Thirty minutes later, mice were euthanized by cervical dislocation and imaged using single photon emission computed tomography (SPECT, MiLabs; resolution: 400 μm resolution) and microCT (ImTek, Inc.; 100 m). SPECT and CT images were imported into ImageJ, processed using a Renyi information entropic filter (Kapur J N et al Graphical Models and Image Processing 1985), and colocalized. Average intensity projections of colocalized SPECT/CT images were generated for a field of view centered on the mouse head. Three dimensional reconstructions were generated in ImageJ's 3D Viewer tool.

The results of the experiments are now described.

### Uniquely Selective and Effective Targeting of Anti-VCAM to the Inflamed Brain

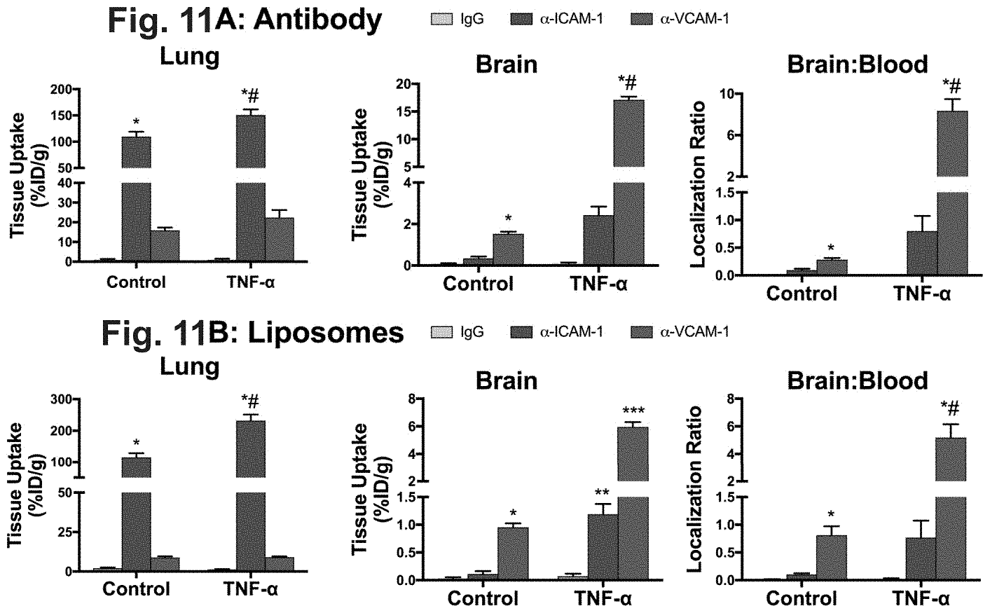

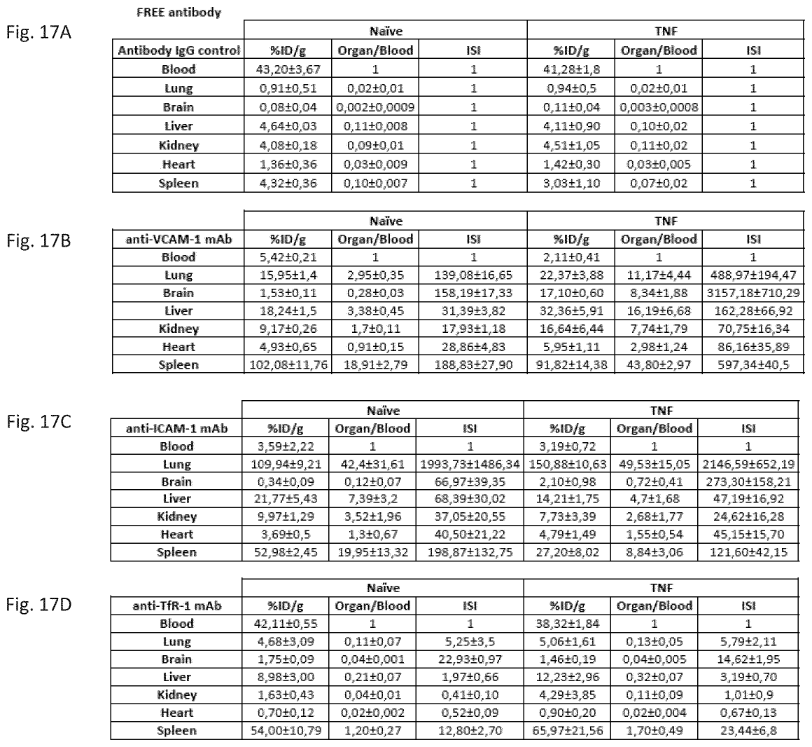

Radiolabeled monoclonal antibodies against VCAM-1, ICAM-1, TfR-1 or control IgG were injected intravenously in control mice and after microinjection of TNFα into the striatum. For each labeled antibody, in vitro immunoreactivity assays were performed prior to quantitative radiotracing to ensure that a high percentage (>80%) retained antigen-binding activity (Bonavia et al., 2006, Appl. Radiat. Isot., 64:470-474). FIG. 11 shows data for the lungs and brain of animals injected with anti-ICAM and anti-VCAM vs. control IgG. Comprehensive data for all ligands and organs for both groups of animals is given in FIG. 17A-FIG. 17D. This large-scale animal study unveiled intriguing and important findings. In control mice, anti-ICAM demonstrated high pulmonary uptake (109.9±9.2 percent of the injected dose per gram of tissue; % ID/g, p<0.001 vs. all other formulations), while uptake of anti-VCAM was an order of magnitude lower (15.91.4% ID/g). Uptake of anti-TfR was three times lower vs anti-VCAM (4.7±3.1, FIG. 17B and FIG. 17D), while that of control IgG was a further five times lower (0.9±0.5% ID/g). In contrast, brain uptake of anti-VCAM in control mice was on par with anti-TfR-1 and four times higher than anti-ICAM (1.5±0.1 vs 1.7±0.1 vs 0.4±0.1% ID/g, p<0.05). Control IgG showed minimal brain uptake (0.1% ID/g, p<0.001 vs. anti-VCAM).

Interestingly, pulmonary uptake of anti-ICAM was further elevated in TNFα-challenged mice compared to naive animals (150.8+10.6% ID/g). This presumably reflects an increase in pulmonary endothelial ICAM-1 and matches the well documented interplay between brain injury and lung inflammation in humans (Hu et al., 2017, Am. J. Physiol.—Lung Cell. Mol. Physiol., 313:L1-L15). In contrast, anti-VCAM uptake in lungs of TNFα-challenged animals showed minimal change (22.3+3.9% ID/g), and pulmonary uptake of anti-TfR and untargeted IgG (5.1+1.6 and 0.9+0.5% ID/g, respectively, FIG. 17B and FIG. 17D) were not changed at all vs. control mice.

Most strikingly and importantly, the uptake of anti-VCAM increased more than an order of magnitude in the brain of TNFα injured mice, far exceeding that of anti-ICAM and anti-TfR (17.1±0.6 vs 2.6±0.2% and 1.5±0.2% ID/g, anti-VCAM, anti-ICAM and anti-TfR respectively, p<0.001). TNFα challenge did not affect IgG uptake in brain (0.1±0.0% ID/g, p<0.001 vs anti-VCAM).

Ratio of % ID/g in an organ and blood (Localization Ratio, LR), allows comparison of different formulations taking into account differences in blood clearance rate. In turn, dividing LR of a targeted formulation by LR of non-targeted one yields the Immunospecificity Index (ISI, FIG. 17A—FIG. 17D). Analysis of these parameters helps one to appreciate the exquisite selectivity and efficacy of anti-VCAM targeting to the inflamed brain. The ISI of anti-VCAM uptake achieved 3157.2±710.3, indicating three orders of magnitude superiority over untargeted IgG, an order of magnitude over anti-ICAM-1 (ISI 273.3±158.2) and more than two orders of magnitude over anti-TfR (ISI 14.6±1.9).

Consistent with the kinetics of induction of expression of VCAM-1 and ICAM-1 in response to TNFα challenge that is known to take hours to reach the maximal expression (Mattila et al., 1992, Scand. J. Immunol., 36:159-65; Burke-Gaffney et al., 1996, Br. J. Pharmacol., 119:1149-1158), targeting to these molecules was time dependent, in contrast with the relatively stable and grossly inferior targeting to TfR.

### Selective Targeting of Anti-VCAM/Liposomes to the Inflamed Brain.

The antibody studies identified VCAM-1 as a uniquely attractive target for inflamed brain. However, parameters of targeting of ligand-coated drug delivery vehicles often differ from those of free ligand, due to different pharmacokinetics, access and binding to the target, among other reasons. To appraise potential drug delivery utility of this finding, this study was reproduced using antibody-coated liposomes, lipid-based nanocarriers that are among the most advanced translational carriers in nanomedicine. Site-specific click chemistry was employed to conjugate antibodies to liposomes, labeled with ¹¹¹In bound to a DTPA-lipid to allow for direct tracing of the liposome.

Intrigued by essentially opposite patterns of cerebral vs pulmonary uptake of antibodies to VCAM-1 and ICAM-1, experiments were focused on targeting liposomes to these two endothelial cell adhesion molecules. In general agreement with the behavior of the antibodies, anti-ICAM/liposomes accumulated extraordinary well in the lungs and the uptake was further elevated in TNFα-striatum challenged mice (115.6 and 231.7±19.5% ID/g) (FIG. 11D), whereas pulmonary targeting of anti-VCAM/liposomes did not exceed 10% ID/g in both control and TNFα-challenged mice and IgG uptake was in the range of 1-2% ID/g in both cases (FIG. 11B).

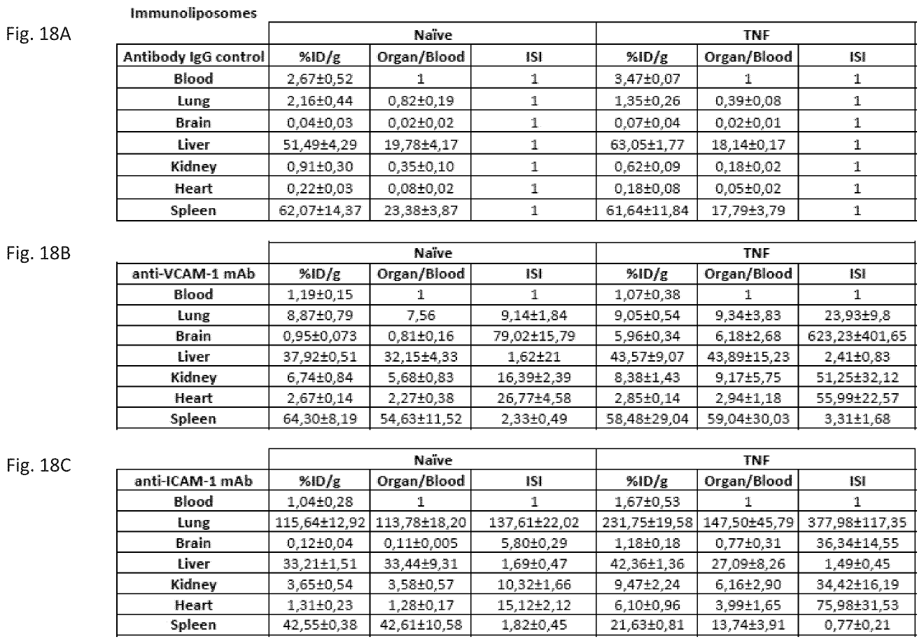

In the brains of control mice, the uptake of anti-VCAM/liposomes was 4 times that of anti-TfR/liposomes (0.2±0.0), and by an order of magnitude exceeded that of anti-ICAM/liposomes and IgG/liposomes (0.9±0.1 vs 0.1±0.0 vs. 0.04±0.03% ID/g, p<0.001 anti-ICAM and IgG/liposomes vs anti-VCAM/liposomes). Furthermore, in mice with TNFα-induced acute brain inflammation, cerebral uptake of anti-VCAM/liposomes reached 6.0±0.3% ID/g, versus 1.2+0.2 and 0.1+0.0% ID/g for anti-ICAM/liposomes and IgG/liposomes, respectively (anti-ICAM and IgG/liposomes vs anti-VCAM/liposomes, p<0.001). The ISI of anti-VCAM/liposomes in the inflamed brain was 623.2±401.6, whereas ISI of anti-ICAM/liposomes was twenty times lower (36.3±14.6; FIG. 18A-FIG. 18C).

### SPECT/CT Imaging of Cerebral Inflammation Using Anti-VCAM-1/Liposomes.

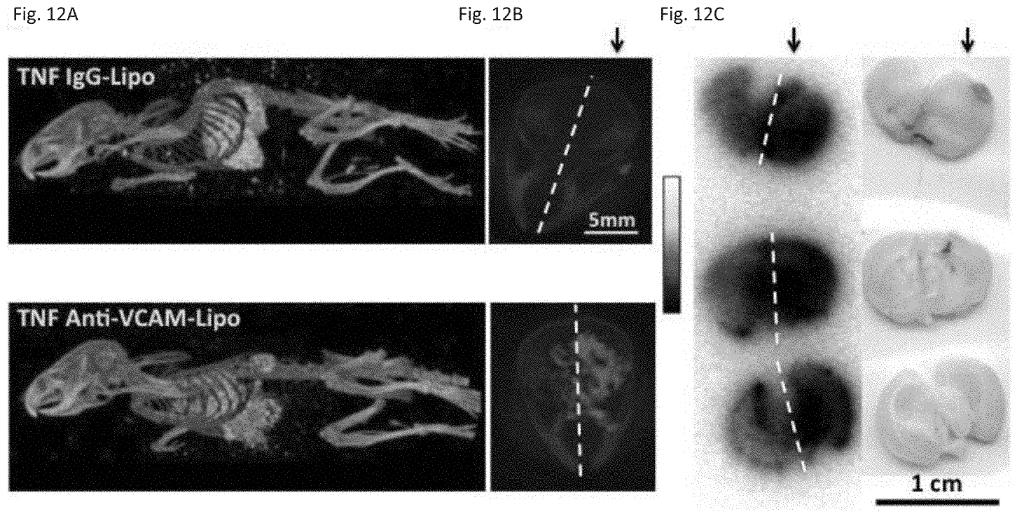

VCAM-1 targeted particles have shown impressive results in imaging of brain inflammation and ischemia using MRI and other modalities in animals (Montagne, et al., Neuroimage 2012, 63, 760-770; Gauberti et al., 2013, Stroke, 44; Patel et al., 2015, Bioconjug. Chem., 26:1542-1549; Liu et al., 2016, Theranostics, 6:1588-1600). Based on the encouraging magnitude of radiolabeled anti-VCAM/liposome uptake in the inflamed brain, experiments were conducted to appraise potential imaging contrast utility of this formulation. Accordingly, anti-VCAM/liposomes or IgG/liposomes were functionalized with DTPA and loaded the chelate with ¹¹¹Indium. ¹¹¹Indium-bearing liposomes were injected intravenously in TNF-injured mice and the mice were imaged with SPECT and CT post-mortem (FIG. 12A).

In contrast to IgG/liposomes, anti-VCAM/liposomes produced a strong signal in the inflamed brain tissue, with signal in the injured hemisphere predominant (FIG. 12B). Autoradiography of 2 mm brain slices from injured mice receiving anti-VCAM/liposomes confirmed the pattern of ¹¹¹Indium signal observed in SPECT images (FIG. 12C). Total background-corrected SPECT signal in the TNF-injured brain was 6-fold higher in mice receiving anti-VCAM/liposomes, as compared to IgG/liposomes. As noted above, other studies have shown that VCAM-targeted nanoparticles can confer specific imaging contrast in injured brain vasculature. However, the present results matches or exceeds previous state of the art in terms of VCAM specificity (Patel et al., 2015, Bioconjug. Chem., 26:1542-1549; Liu et al., 2016, Theranostics, 6:1588-1600), demonstrates VCAM-targeted imaging with SPECT, a quantitative imaging contrast modality (Montagne, et al., 2012, Neuroimage, 63:760-770; Gauberti et al., 2013, Stroke, 44; Frechou et al., 2013, Contrast Media Mol. Imaging, 8:157-164), and employs liposomes, a nanoparticle with demonstrated biocompatibility and translational potential (Patel et al., 2015, Bioconjug. Chem., 26:1542-1549). These results therefore confirm that VCAM-1 targeted nanoparticles can be used for non-invasive imaging of pathological vascular activation in brain pathology.

Intravital Microscopy of Anti-VCAM/Liposome Targeting to Inflamed Brain.

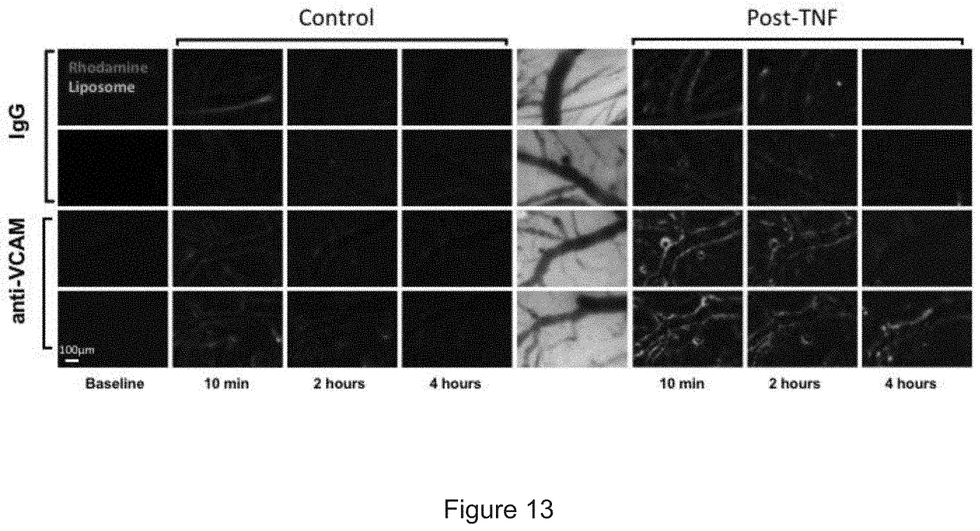

Capitalizing on recent advances in intravital, multi-label fluorescent microscopy of cerebral vasculature in situ via cranial window (Rom et al., 2016, J. Neuroinflammation, 13:254), the real time accumulation of anti-VCAM-1/liposomes was imaged in the inflamed brain. In order to induce inflammation of the superficial cerebral vessels, which can be reliably imaged through the cranial window, TNFα microinjection was administered using a subarachnoid catheter. While no signal was observed at baseline, green fluorescent anti-VCAM/liposomes accumulated at the vascular margin within minutes of intravenous injection in naive mice (FIG. 13, left panels), outlining post-capillary venules. Only occasional leukocytes (labeled with rhodamine dye) were seen passing through the vessels. The liposome signal faded and was no longer visible by 4 hours post-injection.

The same vessels were imaged after a second dose of anti-VCAM/liposomes 24 hours later and 2 hours after localized injection of TNFα (FIG. 13, right panels). In this case, the fluorescent signal was stronger and more prolonged, but the pattern was similar, outlining the vessel walls, consistent with endothelial localization. IgG/liposomes yielded very faint, if discernible at all, fluorescent signal from the cerebral vessels in both control and TNFα-challenged mice.

Leukocytes were abundant in images of the post TNFα-challenged cerebral vasculature (red color in the right images), yet relatively little co-localization was observed with anti-VCAM/liposomes. Indeed, by 4 hours after liposome injection (6 hours post-TNFα infusion), there was essentially no new influx of rhodamine labeled leukocytes, but the green fluorescent signal persisted in what appeared to be the brain endothelium.

### Anti-VCAM and Anti-VCAM/Liposomes are Targeted Predominantly to Endothelial Cells in the Cerebral Vasculature

VCAM-1 is generally considered to be a more specific surface marker of activated endothelial cells than ICAM-1, which is constitutively expressed and further up-regulated during pathology in many cell types (endothelial cells, leukocytes, macrophages, lymphocytes). Yet, studies that would directly attribute localization of VCAM-1 targeted agents to cell types in vivo are lacking.

Fluorescently-labeled antibodies or liposomes were injected in control and TNFα-challenged mice and analyzed by flow cytometry in order to assess the cellular distribution of VCAM-1 targeted agents in the cell suspension obtained from brain homogenates. In control mice, only a small percentage of total cells recovered from brain stained positive for injected anti-VCAM mAb (1.9±0.6%), but an order of magnitude increase in signal was observed in animals challenged with TNFα (15.1±0.5%, p<0.001 vs. naive). In contrast, only 0.6±0.1% of cells (p<0.001) stained positive in TNFα injected animals pre-injected with fluorescent labeled isotype control IgG.

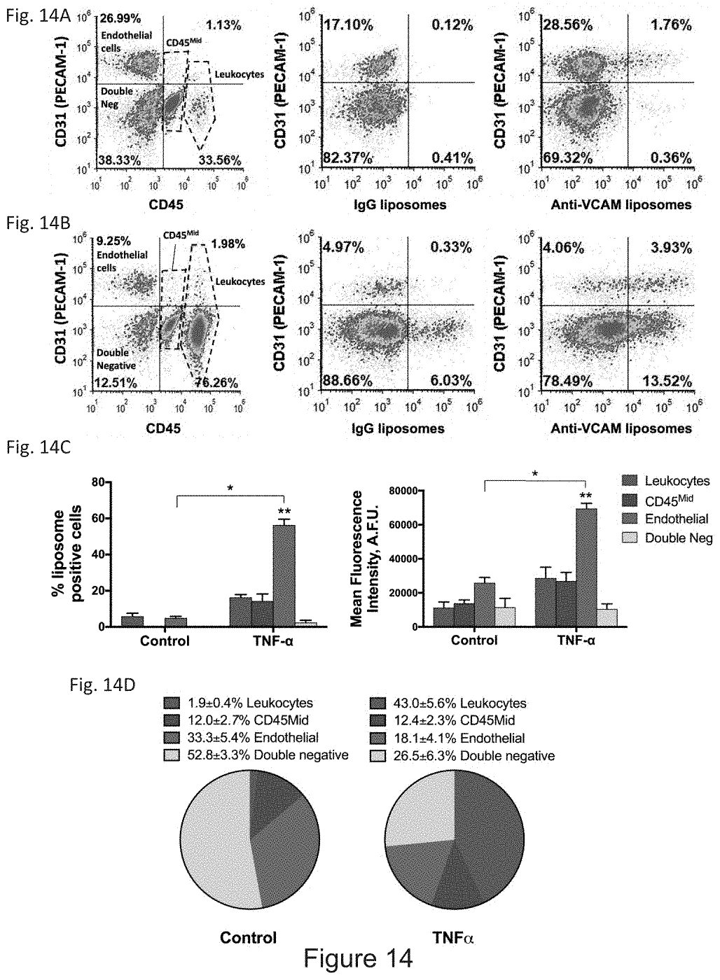

Cells were co-stained for CD31 and CD45 to identify target cells types: endothelial cells (ECs, CD31⁺/CD45^{neg}), leukocytes (CD45^{Hi}) and microglial cells (CD45^{Mid})³³ (FIG. 14). Double negative cells, which stained for neither CD31 nor CD45, were not further sub-typed. The relative percentages of each cell type was fairly consistent for control and TNFα treated animals, but significantly different between the two experimental groups. In the brain of TNFα injected animals, more than half (51.6±1.6%) of recovered ECs were positive for intravenously injected fluorescent anti-VCAM mAb, vs. 10.3±0.7% of leukocytes, 7.4±1.8% of CD45^{Mid}, and 3.1±0.3% CD31-/CD45′ double negative cells (p<0.001 for ECs vs. all other cell types). The mean fluorescence intensity (MFI) on VCAM-1 mAb-positive ECs was also significantly higher (50620±2785 arbitrary fluorescence units) as compared to other cell types (12380±403 for leukocytes, 11542±403 for CD45^{Mid} cells, and 9252±330 for double negative cells, p<0.001 for ECs vs. all other cell types).

Similar results were seen for anti-VCAM/liposomes (FIG. 14A—FIG. 14D). The percentage of total cells recovered that were positive for injected VCAM-1 targeted liposomes was roughly an order of magnitude higher in TNFα injected animals (17.3±2.1%, FIG. 14A) vs. control animals (1.9±0.3%, p<0.001, FIG. 14B). Likewise, 56.3±3.3% of recovered ECs in TNF injected animals were positive for anti-VCAM/liposomes, as compared to leukocytes (16.4±1.5%, p<0.001), CD45^{Mid} cells (14.3±3.9%, p<0.001), and double negative cells (2.5±1.1%) (FIG. 14C). The MFI of endothelial cells was again several-fold higher than that of the other cells types (p<0.001 for ECs vs. leukocytes, CD45^{Mid}, and double negative). Interestingly, injection of IgG/liposomes produced a significantly higher percentage of positive cells in TNFα injected mice (4.6±1.9%) than free IgG (0.6±0.1%, p<0.001), a finding which may be attributed to more effective uptake by Fc-receptor bearing cells, such as infiltrating leukocytes. Indeed, more than 95% of the cells positive for IgG/liposomes were CD45^{Hi} (FIG. 14B).

VCAM-1 Targeted Lipid Nanoparticles (LNP) Carrying Thrombomodulin mRNA Reduce TNFα-Induced Acute Brain Edema

Given the compelling results discussed above, namely the high degree of specific targeting of VCAM-1-targeted agents to inflamed cerebral endothelial cells, it was reasoned that this technology would be particularly well-suited to the delivery of a therapeutic with known activity in endothelial cells. In particular, the endothelial surface glycoprotein thrombomodulin (TM) is known to have critical roles in regulating coagulation, inflammation, and endothelial barrier function, and multiple previous studies have demonstrated its protective effects when targeted to the vascular endothelium (Ding et al., 2009, Am. J. Respir. Crit. Care Med., 180:247-256; Greineder et al., 2017, Blood Adv., 1:1452-1465). Rather than anchor recombinant TM to the luminal membrane of the brain endothelium, a delivery strategy appropriate for non-internalizing surface targets (Murciano et al., 2003, Blood, 101:3977-3984), we utilized a method of targeted gene delivery (LNP mRNA) to induce de novo expression of TM by VCAM-1 expressing cerebrovascular endothelium (Weissman et al., 2015, Molecular Therapy, 23:1416-1417; Pardi et al., 2015, J. Control. Release, 217:345-351).

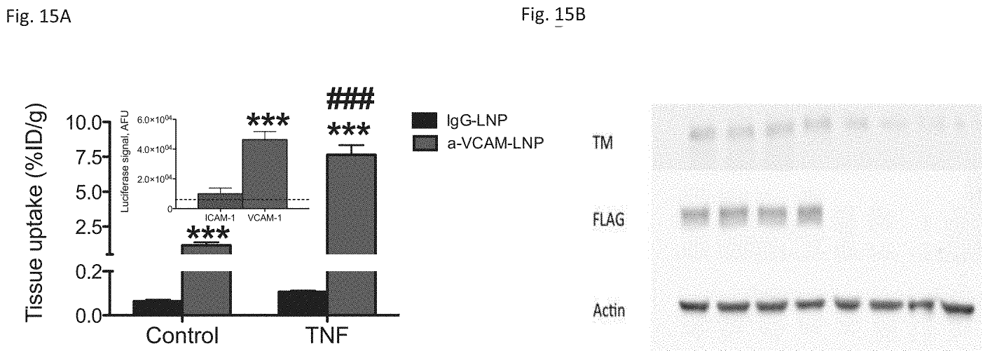

Before testing inducible TM expression, it was confirmed that anti-VCAM/LNP show similar targeting in vivo following intravenous injection of TNF-α. As shown in FIG. 15A, cerebral uptake of radiolabeled anti-VCAM/LNP was >10-fold higher than IgG/LNP in control mice and >70-fold greater in TNFα challenged mice (0.1±0.0 vs 1.1±0.4, and 0.1±0.0 vs 7.6±1.2). Likewise, anti-VCAM/LNPs containing luciferase mRNA induced an almost 5-fold higher level of the reporter signal in the brain of TNFα-challenged mice vs anti-ICAM/LNP and IgG/LNP (FIG. 15A inset).

Next, the expression of TM in brain was tested following injection of anti-VCAM/LNPs loaded with TM mRNA. The TM transgene was tagged such that expressed protein would have a triple-flag tag fused to the cytoplasmic tail of the protein, allowing for straightforward determination of de novo expression vs. endogenous endothelial TM. FIG. 15B shows that mRNA loaded anti-VCAM/LNP, but not anti-ICAM/LNP, induced brain expression of TM when injected 16 hours after TNFα injury (FIG. 15B).

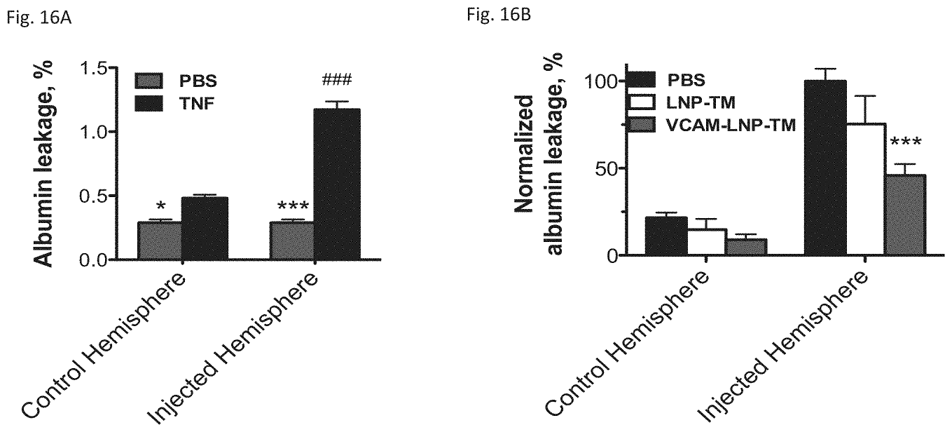

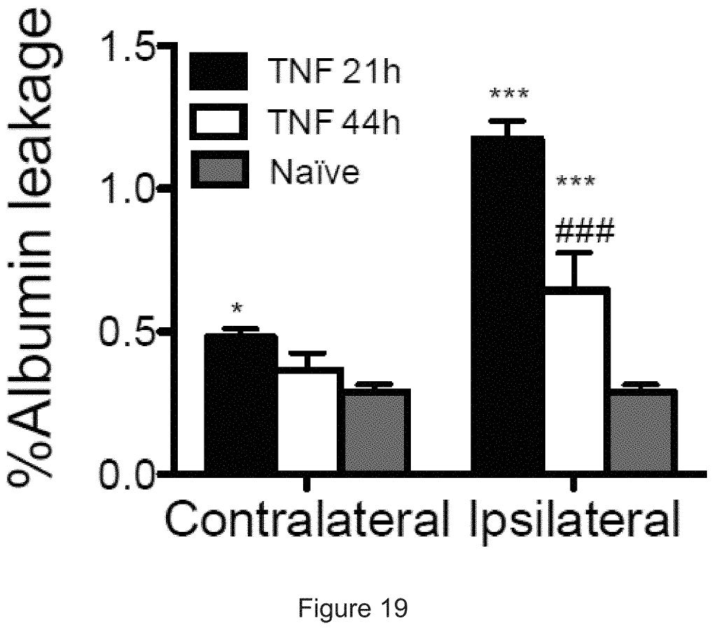

Among other pathological changes typical of cerebral inflammation, TNFα insult also induces brain edema, which we measured by the tissue level of ¹²⁵I-albumin leaking from blood (FIG. 16A). This quantitative readout of vascular permeability rose four times in TNFα challenged animals relative to naive controls (0.29±0.08 vs 1.17±0.23, PBS-injected vs. TNFα-injected animals) and remained unresolved for at least two days (FIG. 19). In a double-blinded study, anti-VCAM/LNPs, but not untargeted LNPs carrying TM mRNA injected after TNFα significantly (p<0.001) alleviated vascular leakage in the brain by 55.1±15.9% for the VCAM-1 targeted LNPs, while it was not significantly reduced for the untargeted LNPs (FIG. 16B).

Inflammatory agents, abnormal blood flow, radiation, trauma, hypoxia, tissue damage, tumor growth and other pathological and injurious factors cause multifaceted abnormalities in endothelial cells, most profoundly in the vicinity of the site of local insult and bifurcations and other vascular sites predisposed to aggravated responses (Kiseleva et al., 2018, Drug Deliv. Transl. Res., 8:883-902; Pober et al., 2007, Nature Reviews Immunology, 7:803-815; Pober et al., 2015, Cold Spring Harb. Perspect. Biol., 7). These pathological changes are manifested by endothelial activation of pro-thrombotic, pro-inflammatory and pro-edematous entities (e.g., cellular contraction, de-encrypting of von Willebrand and tissue factors, exposure of cell adhesion molecules, release of chemokines and cytokines) concomitantly with suppression of endogenous anti-thrombotic and anti-inflammatory mechanisms (e.g., deficiency of endothelial thrombomodulin, decay accelerating factor and CD39) (Coenen et al., 2017, Blood 130:2819-2828; Loghmani et al., 2018, Blood; Iba et al., 2018, Journal of Thrombosis and Haemostasis, 16:231-241). Implications of these endothelial changes include initiation and propagation of pathological pathways. On the other hand, these changes, in theory, can be used for detection and, perhaps, targeting treatment of these disease conditions. To this point, it is demonstrated herein that VCAM-1 targeting to inflamed brain with delivery of TM ameliorates the edematous response.

Analysis of diverse tissue specimen obtained in animal and clinical studies using Western blotting, immunostaining, FACS, functional genomics, PCR and in situ hybridization established that pathologically activated endothelial cells express on their surface VCAM-1, among other inducible surface determinants including selectins (Rossi et al., 2011, J. Leukoc. Biol., 89:539-56). VCAM-1, a cell adhesion molecule of the immunoglobulin super-family, essentially absent in normal adult vasculature, is a more specific marker of abnormal endothelium than other adhesion molecules of this family, ICAM-1 and PECAM-1. Vascular injection of labeled ligands of VCAM-1 provides non-invasive detection of this inducible marker of abnormal endothelia and imaging of vascular pathology in organs including the brain using PET, SPECT, MRI, optical and other modalities (Montagne, et al., Neuroimage 2012, 63, 760-770; Gauberti et al., 2013, Stroke, 44; Nahrendorf et al., 2009, JACC Cardiovasc. Imaging, 2:1213-1222; Patel et al., 2015, Bioconjug. Chem., 26:1542-1549; Tsourkas et al., 2005, Bioconjug. Chem., 16:576-581; Bruckman et al., 2014, Nano Lett., doi:10.1021/n1404816m).

On the other hand, animal studies show that ICAM-1 and PECAM-1 are good targets for prophylactic and therapeutic vascular drug delivery, especially in conditions when massive pharmacotherapy is needed. PECAM-1 is a stable, constitutive, pan-endothelial determinant expressed by endothelia at millions of copies per cell (Howard et al., 2014, ACS Nano 8:4100-32; Brenner et al., 2017, Nanomedicine Nanotechnology, Biol. Med., 13:1495-1506). ICAM-1 is abundantly and constitutively expressed in many vascular beds, especially in the lungs, and further up-regulated in pathological states (Danielyan et al., 2007, J. Pharmacol. Exp. Ther., 321:947-52).

Comparison of targeting features of these adhesion molecules illustrates one of the conundrums of attempts to devise carriers for simultaneous delivery of therapeutic and imaging agents, so called “theranostics”. While utmost selectivity and target/non-target ratio are top priorities for imaging, the top priorities in drug targeting is to deliver an effective dose precisely to the site of action. It was examined herein whether VCAM-1 targeted therapeutics could provide beneficial effects in cerebral inflammation. The fact that the density of expression of VCAM-1 on the surface of abnormal endothelia is orders of magnitude lower than that of ICAM-1 reduced enthusiasm. In this study, the first direct and quantitative systematic analysis of the uptake of injected isotope-labeled antibodies to candidate molecular targets of inflamed cerebrovascular endothelium in organs of control mice and mice with acute brain inflammation was performed. Unexpectedly, it is found that anti-VCAM agents uniquely afford both high electivity and efficacious drug delivery to the inflamed brain.

The present data shows that, in some key aspects, VCAM-1 and ICAM-1 have opposite targeting features. Uptake of anti-ICAM and anti-ICAM/agents in lungs exceeds that of anti-VCAM counterparts by orders of magnitude. In contrast, uptake in the inflamed brain of anti-VCAM formulations exceeds that of anti-ICAM counterparts by a similar margin. Despite the fold change difference between anti-ICAM and anti-VCAM agents is similarly in control and challenged animals, anti-VCAM agents accumulate at higher levels selectively in the inflamed brain. While not wishing to be bound by any particular theory, it is plausible that anti-VCAM cerebrovascular targeting is enabled by low level of competitive binding in the lungs and likely other peripheral vascular areas. The immunotargeting of anti-VCAM agents to the inflamed brain is specific (IgG counterparts show very low basal levels in both organs) and exceeds by two orders of magnitude that of anti-TfR1, one of the most commonly used ligands for cerebral targeting (FIG. 17A—FIG. 17D).

These findings, for the first time, establish that vascular immunotargeting to VCAM-1 enables a uniquely selective and effective drug delivery to abnormal cerebral endothelium. This novel approach, in theory, may help: I) Accumulate drug in desirable sites of action; II) Guide precise cellular and sub-cellular addressing of drug; III) Interfere with functions of target molecules and cells.

This study opens new avenues for investigation of the effects, medical utility and mechanisms of cerebral targeting to VCAM-1. Local and migrant constituents of CNS pathologies are immensely diverse. No single imaginable drug delivery system can optimally address the full range of spatiotemporal delivery applications. However, the present results strongly support the notion of potential utility of VCAM-1 targeting for precise therapeutic interventions of CNS pathologies. The ability to non-invasively detect pathologic changes in the brain is likely to provide mechanistic, diagnostic and prognostic insights in challenging conditions, such as stroke, intracranial hemorrhage, cerebral inflammation, among other grave conditions.

The neuroinflammatory response to initial injury is both a potential cause of secondary neuronal damage and a potential therapeutic target. While anti-inflammatory therapies have failed to show benefit in clinical trials, relatively few attempts have been made to direct therapeutics specifically to areas of cerebrovascular activation or pathology. It is demonstrated herein that VCAM-1 targeting can deliver genetic therapies resulting in the lessening of pathologic abnormalities in the inflamed brain that are responsible for short and long-term damage and resulting debility.

### Example 3: PECAM-1 Directed Re-Targeting of Exogenous mRNA Providing Two Orders of Magnitude Enhancement of Vascular Delivery and Expression in Lungs Independent of Apolipoprotein E-Mediated Uptake

Systemic administration of lipid nanoparticle (LNP)-encapsulated messenger RNA (mRNA) leads predominantly to hepatic uptake and expression. Here, experiments were conducted where nucleoside-modified mRNA-LNPs were conjugated with antibodies (Abs) specific to vascular cell adhesion molecule, PECAM-1. Systemic (intravenous) administration of Ab/LNP-mRNAs resulted in profound inhibition of hepatic uptake concomitantly with ˜200-fold and 25-fold elevation of mRNA delivery and protein expression in the lungs compared to non-targeted counterparts. Unlike hepatic delivery of LNP-mRNA, Ab/LNP-mRNA is independent of apolipoprotein E. Vascular re-targeting of mRNA represents a promising, powerful, and unique approach for novel experimental and clinical interventions in organs of interest other than liver.

Messenger RNA (mRNA)-based therapeutic approaches emerged as alternative treatment options in the fields of vaccination, protein replacement therapy, and cellular reprogramming (Sahin et al., 2014, Nat. Rev. Drug Discov., 13:759-780; Pardi et al., 2018, Nat. Rev. Drug Discov., doi:10.1038/nrd.2017.243). One of the most promising delivery platforms is nucleoside-modified and purified mRNA encapsulated in lipid nanoparticles (LNPs) (Pardi et al., 2015, J. Control. Release. 217:345-351). Nucleoside modification and HPLC purification of the mRNA are important to increase protein production in vivo and eliminate inflammatory responses after administration (Karikó et al., 2008, Mol. Ther., 16:1833-1840; Karikó et al., 2011, Nucleic Acids Res.). LNPs containing ionizable amino lipids are employed to pack mRNA and protect cargo en route to the site of action (Kauffman et al., 2016, J. Control. Release., 240:227-234). It has been recently demonstrated that administration of antibody-encoding mRNA-LNPs resulted in high levels of functional antibodies that protected mice from infectious pathogens (Pardi et al., 2017, Nat. Commun., 8:14630; Thran et al., 2017, EMBO Mol. Med., 9:e201707678), and toxins (Thran et al., 2017, EMBO Mol. Med., 9:e201707678, as well as increased tumor clearance in murine models (Thran et al., 2017, EMBO Mol. Med., 9:e201707678; Stadler et al., 2017, Nat. Med. 23:815-817). In addition to potency, mRNA has several beneficial features over other therapeutic delivery platforms, such as the inability to integrate into the host genome, and transient and controllable translation in cells.

Organ and cell type-specific delivery of mRNA after systemic administration is a major challenge. Upon systemic delivery, mRNA-LNPs mainly target the liver due to their ability to bind apolipoprotein E (apoE) and target apoE receptors on the surface of hepatocytes (Akinc et al, 2010, Mol. Ther., 18:1357-1364). Coupling affinity ligands, such as antibodies to specific target molecules, to the surface of nanocarriers provides an alternative approach for targeted delivery. Affinity targeting may provide more precise control of distribution in an organ and destination in the target cells (Shuvaev et al., 2015, J. Control. Release., 219:576-595).

Endothelial cells lining the vascular lumen represent targets for pharmacological interventions in many cardiovascular, neurological, and pulmonary conditions (Shuvaev et al., 2015, J. Control. Release., 219:576-595; Aird, 2003, Blood., 101:3765-3777; Maniatis et al., 2008, Curr. Opin. Crit. Care., 14:22-30; Thorpe et al., 2004, Clin. Cancer Res., 10:415-427). Endothelial targeting of diverse agents and carriers to the pulmonary, cerebrovascular, and other vascular areas has been achieved using antibodies and other affinity ligands that bind endothelial surface determinant CD31 (aka platelet-endothelial cell adhesion molecule-1 (PECAM-1), among others (Han et al., 2012, Ther. Deliv., 3:263-276; Howard et al., 2014, ACS Nano., 8:4100-4132; Spragg et al., 1997, Proc. Natl. Acad. Sci. U.S.A, 94:8795-8800; Nowak et al., 2010, Eur. J. Cardio-Thoracic Surg., 37:859-863; Khoshnejad et al., 2016, Bioconjug Chem., 27:628-637; Albelda, 1991, Am. J. Respir. Cell Mol. Biol., 4:195-203). Described herein is the potent vascular targeting of nucleoside-modified and fast protein liquid chromatography (FPLC)-purified mRNA to the lungs using LNP-mRNA-coupled PECAM-1 antibodies in mice.

The materials and methods employed in these experiments are now described.

Reagents

N-succinimidyl S-acetylthioacetate (SATA) was purchased from Pierce Biotechnology (Rockford, Ill.). Radioactive isotope ¹²⁵I was purchased from Perkin-Elmer (Wellesley, Mass.). Whole molecule rat IgG was from ThermoFisher (Waltham, Mass.). Anti-mouse-PECAM-1/CD31 monoclonal antibody was obtained from BioLegend (San Diego, Calif.). Monoclonal antibodies to human PECAM-1 (anti-PECAM, Ab62) were obtained (Centocor) (Gurubhagavatula et al., 1998, J. Clin. Invest., 101:212-222). All chemical reagents were purchased from Sigma Aldrich, unless stated otherwise.

Cell Culture

Human mesothelioma REN cells, either stably expressing human PECAM-1 (REN-PECAM) or not (REN wild type), have been previously described (Sun et al., 1996, J. Biol. Chem., 271:18561-18570; Sun et al., 2000, J. Cell Sci., 113:1459-1469; Delisser et al, 1994, J. Cell Biol., 124:195-203; Jackson et al., 1997, J. Biol. Chem., 272:24868-24875). REN cells were maintained in RPMI 1640 supplemented with 10% FBS, 2 mM L-glutamine, 100 U/mL penicillin, and 100 μg/mL streptomycin (Life Technologies, Carlsbad, Calif.). Maintenance media for REN-PECAM cells also contained Geneticin (G418) at 200 μg/mL, as a selection antibiotic.

Human umbilical vein endothelial cells (HUVECs), purchased at passage 1 from Lonza (Walkersville, Md.) were subcultured up to six passages in endothelial basal medium (EBM) supplemented with EGM-bulletkit (Lonza). Passages between 4 and 6 were used throughout the studies.

mRNA Production and Formulation into Lipid Nanoparticles

mRNAs were produced, as described previously (Pardi et al., 2013, In vitro transcription of long RNA containing modified nucleosides, in: P. M. Rabinovich (Ed.), Synth. Messenger RNA Cell Metab. Modul. Methods Protoc., Humana Press, Totowa, N.J., 29-42), using T7 RNA polymerase (Megascript, Ambion) on linearized plasmids encoding codon-optimized firefly luciferase (pTEV-Luc2-A101) and eGFP (pTEV-eGFP-A101). To make modified nucleoside-containing mRNA, m1Ψ-5′-triphosphate (TriLink) was incorporated instead of UTP. mRNAs were transcribed to contain 101 nucleotide-long poly(A) tails. They were capped using the m7G capping kit with 2′-O-methyltransferase (ScriptCap, CellScript) to obtain cap1. mRNA was purified by Fast Protein Liquid Chromatography (FPLC) (Akta Purifier, GE Healthcare) (Weissman et al., 2013, HPLC purification of in vitro transcribed long RNA, in: P. M. Rabinovich (Ed.), Synth. Messenger RNA Cell Metab. Modul. Methods Protoc., Humana Press, Totowa, N.J., 43-54). All prepared RNAs were analyzed by electrophoresis using denaturing or native agarose gels and stored at −20° C.

FPLC-purified m1Ψ-containing firefly luciferase and eGFP-encoding mRNAs were encapsulated in LNPs using a self-assembly process in which an aqueous solution of mRNA at pH=4.0 is rapidly mixed with a solution of lipids dissolved in ethanol (Maier et al., 2013, Mol. Ther., 21:1570-1578). LNPs used in this study were similar in composition to those described previously (Maier et al., 2013, Mol. Ther., 21:1570-1578; Jayaraman et al., 2012, Angew. Chem. Int. Ed. Engl., 51:8529-8533), which contain an ionizable cationic lipid/phosphatidylcholine/cholesterol/PEG-lipid (50:10:38.5:1.5 mol/mol) and were encapsulated at an RNA to total lipid ratio of ˜0.05 (wt/wt). They had a diameter of ˜80 nm as measured by dynamic light scattering using a Zetasizer Nano ZS (Malvern Instruments Ltd, Malvern, UK) instrument. mRNA-LNP formulations were stored at −80° C. at a concentration of mRNA of ˜1 μg/μl.

Preparation and Characterization of Targeted Lipid Nanoparticles

LNPs were conjugated with mAb specific for PECAM-1. Targeting antibodies or control isotype-matched IgG were conjugated to LNP particles via SATA-maleimide conjugation chemistry (Howard et al., 2015, Mol. Pharm., 11:2262-2270). The LNP construct was modified with maleimide functioning groups (DSPE-PEG-mal) by a post-insertion technique with minor modifications (Ishida et al., FEBS Lett. 460 (1999) 129-133). The antibody was functionalized with SATA (N-succinimidyl S-acetylthioacetate) (Sigma-Aldrich) to introduce sulfhydryl groups allowing conjugation to maleimide. SATA was deprotected using 0.5 M hydroxylamine followed by removal of the unreacted components by G-25 Sephadex Quick Spin Protein columns (Roche Applied Science, Indianapolis, Ind.). The reactive sulfhydryl group on the antibody was then conjugated to maleimide moieties using thioether conjugation chemistry. Purification was carried out using Sepharose CL-4B gel filtration columns (Sigma-Aldrich). mRNA content was calculated by performing a modified Quant-iT RiboGreen RNA assay (Invitrogen).

Size and surface charge of the mRNA containing lipid nanoparticles was determined using dynamic light scattering (DLS) and laser doppler velocimetry (LDV), respectively on a Malvern Zetasizer Nano ZS (Malvern Instruments, Worcestershire, UK). For size measurements, LNPs were diluted in PBS pH 7.4 at 25° C. in disposable capillary cuvettes. A non-invasive back scatter system (NIBS) with a scattering angle of 1730 was used. Diameters of unconjugated and antibody-modified particles were interpreted as normalized intensity size distribution as well as z-average values for particle preparations. Zeta potential measurements were also carried out in PBS buffer using disposable folded capillary cells. To assess the stability of antibody-modified LNPs, the particles were incubated in a range of 0-1000 mM NaCl solution and their hydrodynamic diameters were measured on DLS.

Morphologic characterization was carried out on a JEOL1010 transmission electron microscope (TEM), as described (Khoshnejad et al., 2016, Bioconjug Chem., 27:628-637). Briefly, carbon-coated 200-mesh copper grids were placed on a drop of the sample for 2 min and washed with Milli-Q water. Negative staining was done using 2% uranyl acetate. The stain was then wicked off with a filter paper and the grids were dried and imaged at an acceleration voltage of 120K.

In Vitro Cell Binding Assay with Radiolabeled Particles

LNPs were first radiolabeled with Na¹²⁵I using Iodination Beads (Pierce). The reaction was performed for 15 min at room temperature. Unreacted materials were then removed by Quick Spin Protein Columns (G-25 Sephadex, Roche Applied Science, Indianapolis, Ind.) (Khoshnejad et al., 2016, Bioconjug Chem., 27:628-637). Anti PECAM-1 targeted LNP antibody conjugation was evaluated by incubation on REN-PECAM-1 cells, which stably express PECAM-1. Wild-type REN cells, a human mesothelial cell line that has no endogenous expression of PECAM-1, was tested in parallel to assess non-specific binding of particles. REN cells were incubated with increasing quantities of LNPs for one hour at room temperature. Incubation medium was then removed and cells were washed with PBS buffer three times to remove the unbound nanoparticles from the cell surface. The cells were lysed with 1% Triton X100 in 1 N NaOH and the cell-associated radioactivity was measured by a Wallac 1470 Wizard gamma counter (Gaithersburg, Md.) and compared to total added activity.

In Vitro Cell Transfection with Reporter mRNA-Loaded LNPs

REN cells were seeded in 48-well plates. After 18 hours, LNPs carrying reporter firefly luciferase mRNA were added at increasing concentrations to the cells, and incubated for 1.5 hours. Plates were then washed three times with PBS and complete medium was added to the cells. After culturing for 24 hours in complete media, cells were washed with PBS, lysed in luciferase cell culture lysis reagent (Promega, Madison, Wis.) and the firefly luciferase enzymatic activity as luminescence (Luciferase assay system, Promega) was measured. Transfections were performed in triplicate.

For fluorescence microscopy, REN cells or HUVECs were plated at 150,000 cells per well in 24-well plates. At ˜70% cell confluence, LNPs carrying eGFP mRNA were added to the media as 6 μg mRNA per well and cells were incubated for 18 hours. The level of eGFP production was then evaluated by imaging the cells under an EVOS-FL imaging system (Thermofisher scientific, Waltham, Mass.).

Cell Metabolic Activity (MTS Assay)

Cell metabolic activity (MTS assay) was performed on REN cells after incubating with LNPs. Cells were seeded at the same density as luciferase assay and were incubated with a relevant concentration range of particles for 4.5 hours. MTS reagent was added to the wells based on manufacturer's recommendation (MTS Assay Kit, abcam, Cambridge, Mass.), incubated for 2 hours at 37° C. and the absorbance was read at 490 nm.

Detection of Expression of Inflammation Marker, Endothelial Vascular Cell Adhesion Molecule (VCAM)

Cell lysates were first run on sodium dodecyl sulfate polyacrylamide gel electrophoresis (SDS-PAGE), 4-15% gradient gel (Mini-PROTEAN® TGXTM Gel, Bio-Rad, Hercules, Calif.). After gel transfer to PVDF membrane (Millipore, Billerica, Mass.), membrane was blocked with 3% non-fat dry milk in TBS-T (100 mM Tris, pH 7.5; 150 mM NaCl; 0.1% Tween 20) and was incubated with the corresponding antibodies. The blot was detected using ECL reagents (GE Healthcare, New York, N.Y., USA).

Pharmacokinetics/Biodistribution Studies Upon Intravenous Injection of Radiolabeled LNP-mRNAs in Mice

Radiolabeled LNP-mRNAs were administered by retro-orbital injection in C57BL/6 mice (The Jackson Laboratory, Bar Harbor, Me.). Animals were sacrificed at 5, 15, 30, and 60 minutes post-injection and their blood was collected from the inferior vena cava. Organs (liver, spleen, lung, kidney, heart, and brain) were harvested, rinsed with saline, blotted dry, and weighed. Tissue radioactivity in organs and 100-μl samples of blood was measured in a gamma counter (Wallac 1470 Wizard gamma counter, Gaithersburg, Md.). Radioactivity values and weight of the samples were then used to calculate targeting parameters of nanoparticles, including tissue uptake as percent of injected dose per gram tissue (% ID/g), and localization ratio (LR) as organ-to-blood ratio. Immunospecificity index (ISI) was also calculated as the ratio of the LR of targeted particles to that of non-targeted (IgG) control. These parameters were employed to discuss biodistribution and effectiveness of antibody-targeted formulation uptake in desired tissue.

Functional Activity—Firefly Luciferase Transfection In Vivo

Mice (The Jackson Laboratory, Bar Harbor, Me.) were intravenously injected with unconjugated or antibody-conjugated LNP-mRNA formulations. At desired time points, animals were euthanized and all the vital organs were resected, washed with PBS, and stored at −80° C. until analysis.

Organ samples were homogenized in 1 ml of cell lysis buffer (1×) (Promega Corp, Madison, Wis.) containing protease inhibitor cocktail (1×) and mixed gently at 4° C. for one hour. The homogenates were then subjected to cycles of freeze/thaw in dry ice/37° C. The resulting cell lysate was centrifuged for 10 min at 16,000 g at 4° C. Luciferase activity was assayed in the supernatant using a Victor³ 1420 Multilabel Plate Counter (Perkin Elmer, Wellesley, Mass.)

Cytokine Measurement

Mice (The Jackson Laboratory, Bar Harbor, Me.) were intravenously injected with antibody-conjugated LNP-mRNA formulations as described above. At 4.5 hrs post-treatment, animals were euthanized and blood samples were collected in heparin and spun at 1500×g for 10 min at 4° C.

Livers were also collected and homogenized in 1 ml of PBS containing protease inhibitor cocktail (1×). Lysis buffer was added to the homogenates and mixed at 4° C. for one hour. The cell lysate was centrifuged for 10 min at 16,000 g at 4° C. The levels of macrophage inflammatory protein 2 (MIP-2) cytokine in liver and Interleukin 6 (IL-6) in plasma were assessed by ELISA according to manufacturer's protocol (DuoSet ELISA kits, R and D systems, Minneapolis, Minn.).

Bioluminescence Imaging

Bioluminescence imaging was performed as described previously (Pardi et al., 2015, J. Control. Release., 217:345-351) using an IVIS Spectrum imaging system (Caliper Life Sciences, Waltham, Mass.). Mice were administered an intraperitoneal injection of D-luciferin at a dose of 150 mg/kg. After 5 min, the mice were euthanized; organs were quickly harvested, and placed on the imaging platform. Organ luminescence was measured on the IVIS imaging system using an exposure time of 5 s or longer to ensure that the signal obtained was within operative detection range. Bioluminescence values were also quantified by measuring photon flux (photons/second) in the region of interest using LivingImage software provided by Caliper.

Flow Cytometry

LNPs containing DIO-tagged lipids were conjugated to antibodies, as described above. Thirty minutes after IV administration of antibody-modified fluorescent LNPs into mice, the lungs were perfused and harvested. Briefly, the lungs were first digested and cell suspensions were passed through 100 m nylon strainer. To lyse RBCs, ACK lysis buffer (Quality Biological, Gaithersburg, Md.) was used. Anti-CD31, anti-CD45, and anti-F4/80 were used for staining endothelial cells, leukocytes, and macrophages/monocytes, respectively. Flow cytometry was performed on Accuri C6 instrument (Becton Dickinson, San Jose, Calif.).

Statistical Analysis

Unless specified otherwise, the data have been calculated and presented as mean±standard error of mean (SEM). When comparing two groups, a Student's t-test was used assuming a Gaussian distribution with unequal variances. All probability values are two-sided, and values of p<0.05 were deemed statistically significant.

The results of experiments are now described.

### Characterization of Targeted LNP-mRNAs

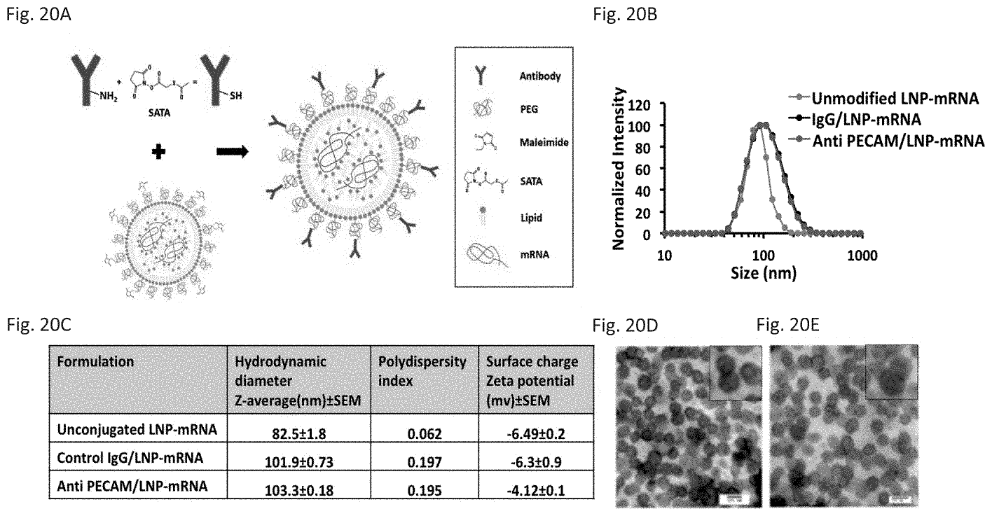

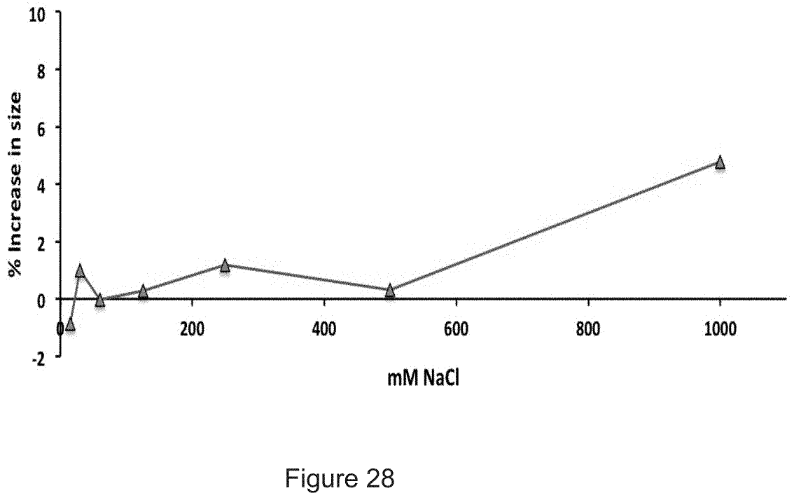

Ab/LNP-mRNA complexes assembled as illustrated (FIG. 20A) have physicochemical properties generally similar to those of unconjugated LNP-mRNAs. Dynamic light scattering revealed a hydrodynamic diameter of 82.5±1.8 nm with narrow size distribution (PDI=0.062) for unconjugated LNP-mRNA. Upon coupling antibody to LNPs, the mean z-average of particles increased up to ˜100 nm (for example, 101.9±0.7 nm and 103.3±0.2 nm for IgG/LNP-mRNA and PECAM-1 Ab/LNP-mRNA, respectively, with PDI ˜0.2) (FIG. 20B and FIG. 20C). Incubation of antibody-conjugated LNPs in a range of ionic strength solutions did not affect the particle size (FIG. 28) demonstrating robust stability of antibody-conjugated LNPs. Moreover, conjugation of IgG and antibodies did not affect the zeta-potential and morphology of the nanoparticles (FIG. 20C—FIG. 20E). Transmission electron microscopic analyses of unconjugated (FIG. 20D) and PECAM-1-conjugated LNP-mRNAs (FIG. 20E) demonstrated maintenance of morphology.

To evaluate antibody conjugation efficiency to LNPs, radiolabeled antibodies were monitored throughout conjugation steps. Quantified radioactivity of the conjugated antibody to LNPs, corresponded to ˜80 antibody molecules per particle.

### Targeting to Endothelial Cells In Vitro

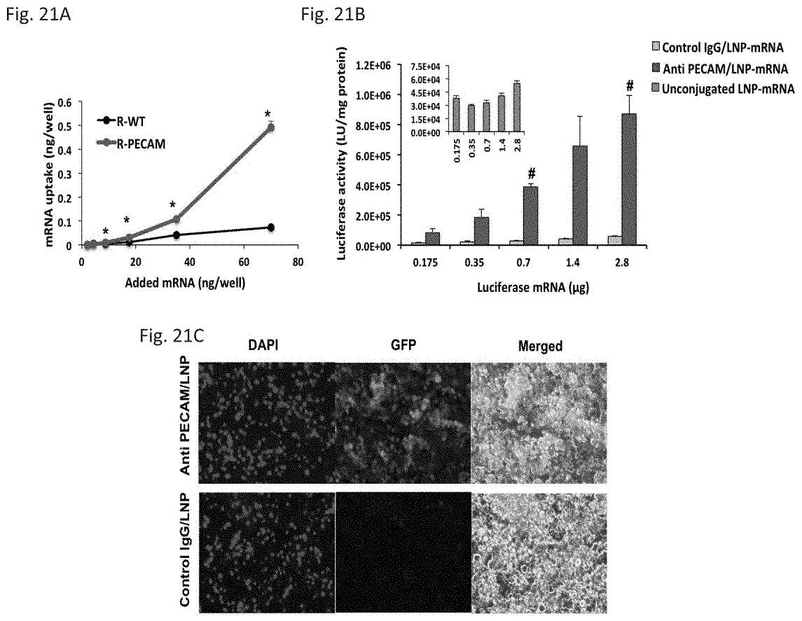

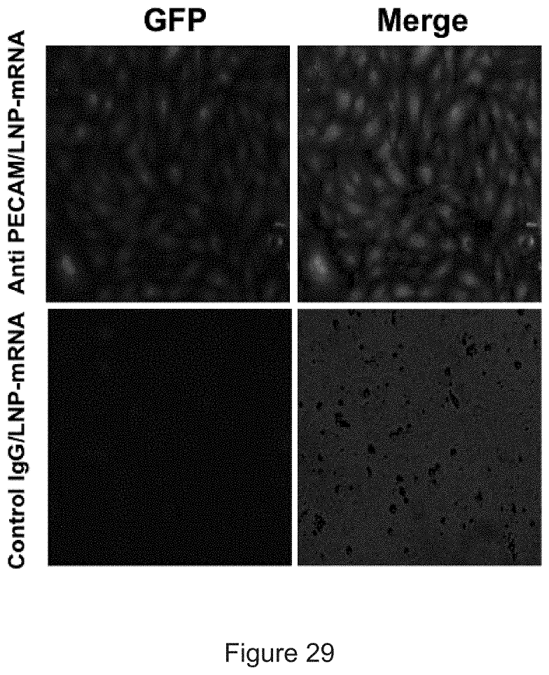

CD31 (PECAM-1) is mainly expressed by the endothelium and is mostly localized on the endothelial intercellular junctions (Scherpereel et al., 2002, J. Pharmacol. Exp. Ther., 300:777-786). PECAM-targeted Ab/LNP-mRNA bound to REN cells transfected with human PECAM-1 (REN-PECAM (Gurubhagavatula et al., 198, J. Clin. Invest., 101:212-222), but not to wild type REN cells (FIG. 21A). This led to the PECAM-dependent expression of the reporter proteins firefly luciferase (FIG. 21B) and enhanced green fluorescent protein (eGFP) (FIG. 21C and FIG. 29) encoded by the mRNA.

Targeting of mRNA-LNPs to Vascular Endothelium after Systemic Delivery in Mice

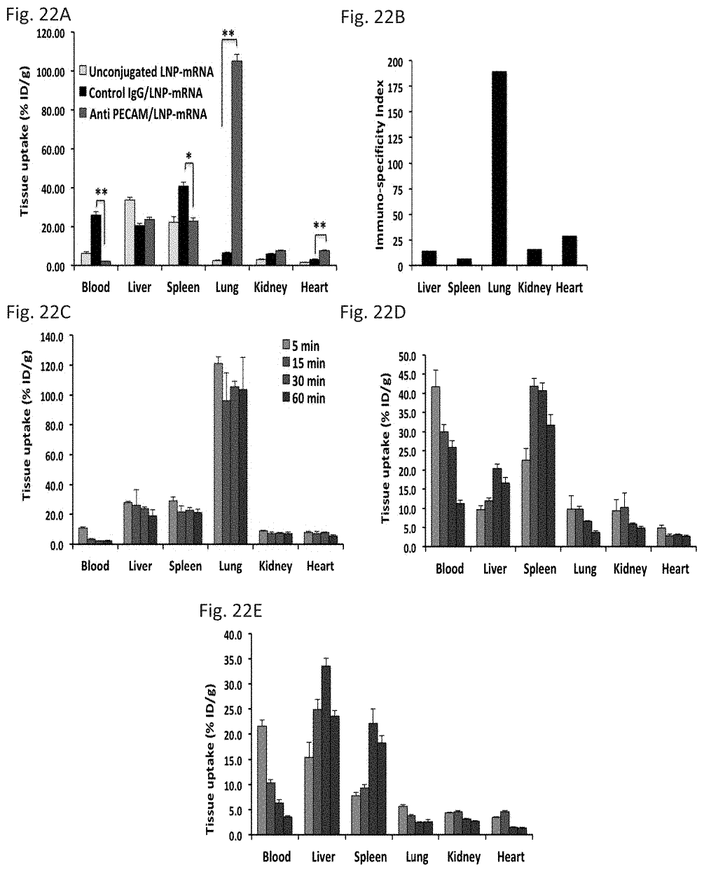

Next, experiments were conducted to study tissue binding of LNP-mRNA formulations directly labeled with ¹²⁵I prior to antibody conjugation, by determining the percent of injected dose per gram of tissue (% ID/g) in mice after intravenous administration. Consistent with previous reports (Pardi et al., 2015, J. Control. Release., 217:345-351; Maier et al., 2013, Mol. Ther., 21:1570-1578), unconjugated LNP-mRNAs accumulated mainly in the liver (FIG. 22A) and to a lesser extent in the spleen. Comparing with pristine LNP-mRNA, IgG/LNP-mRNA showed higher level in blood and spleen, which squares well with concomitant reduction of IgG/LNP-mRNA level in liver (FIG. 22A). It is likely that elevation of blood level due to direct and/or indirect inhibition of hepatic uptake by IgG coat allows IgG/LNP-mRNA to accumulate in the organs and cells that otherwise are outcompeted by massive uptake in liver. On the other hand, since IgG/LNP-mRNA has no affinity to the major target such as endothelium, there is no blood depletion unlike anti PECAM/LNP-mRNA that exerts prominent accumulation in the lungs.

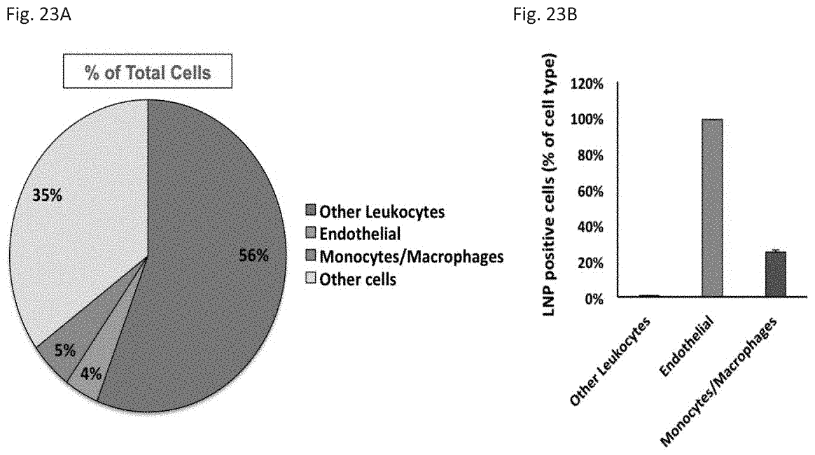

PECAM-1 provided targeting for vascular delivery to the lungs (Khoshnejad et al., 2016, Bioconjug Chem., 27:628-637; Muro et al., 2006, J Pharmacol Exp Ther., 317:1161-1169; Bhowmick et al., 2012, J. Control. Release., 157:485-492). Pulmonary uptake of PECAM-1 targeted Ab/LNP-mRNA reached 105.03±3.5% ID/g (FIG. 22A), providing a 16-fold increase compared to IgG/LNP-mRNA. To compare Ab/LNP-mRNA with IgG/LNP-mRNA, an immunospecificity index (ISI, the ratio of the % ID/g of these formulations in a given organ, normalized to blood level) was calculated. According to this index, comparing these two nearly identical formulations, targeting affords ˜200-fold enhancement of delivery to the pulmonary vasculature (FIG. 22B). Kinetic studies revealed that ¹²⁵I-labeled unconjugated LNP-mRNA was quickly cleared from blood, reflecting uptake in liver and spleen (FIG. 22E). Blood clearance of PECAM-Ab/LNP-mRNA was even more expeditious, but reflected mostly pulmonary uptake (FIG. 22C). Specific lung uptake of PECAM-Ab/LNP-mRNA was rapid and sustained through the last time point studied (60 minutes post-injection) (FIG. 22C). To assess the cellular distribution of PECAM-1 targeted LNPs in vivo, DIO-labeled LNPs were conjugated to anti-PECAM-1 and injected i.v. in mice. Flow cytometry was then performed on the cell suspension obtained from lung homogenates. Cells were co-stained with antibodies against CD31, an endothelial cell marker; CD45, a leukocyte marker; and F4/80 a monocyte/macrophage marker. Only a small percentage (4%) of total cells recovered from the lung were stained positive for CD31 (FIG. 23A). However, 100% of the recovered endothelial cell population were positive for LNPs, showing high uptake of targeted particles by endothelial cells.

### In Vitro and In Vivo Toxicity/Inflammatory Studies

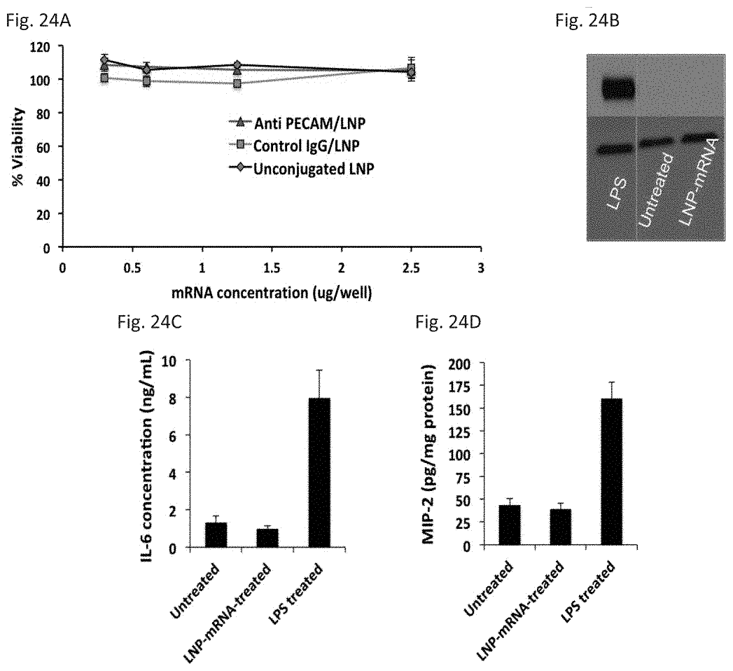

To evaluate the effect of unconjugated and Ab/conjugated LNPs on cellular metabolic activity, MTS assays were performed on REN cells upon LNP treatment. As shown in FIG. 24A, incubation of cells with LNPs for 4.5 hours did not lower the viability of cells below 97% for any of the constructs at tested concentrations.

Pro-inflammatory conditions can induce the expression of leukocyte adhesion molecules such as VCAM-1 and ICAM-1 (Shuvaev et al., 2011, FASEB J., 25(1):348-357; Thomas et al., 2008, Antioxid. Redox Signal., 10(10):1713-1766). To assess if LNP-mRNA treatment might induce pro-inflammatory markers, we analyzed VCAM expression on HUVECs after incubation with LNPs. Cell lysates from HUVECs were collected 4.5 hours after LNP incubation. The expression of VCAM was analyzed by western blot (FIG. 24B). LNP-mRNA treatment did not induce VCAM expression, while the positive control (LPS, 0.5 μg/mL) showed strong VCAM expression.

To determine if LNP-mRNA treatment might affect pro-inflammatory cytokine profile in vivo, plasma and liver tissues were collected from mice 4.5 hours after i.v. administration of LNP-mRNA (8 μg/mouse). Plasma or liver tissue from mice challenged with intravenous LPS (2 mg/kg) was used as a positive control. IL-6 in plasma and MIP-2 in liver did not elevate upon LNP-mRNA treatment in vivo (FIG. 24C), when compared to untreated mice.

Tissue Transfection Pattern after i.v. Administration of Targeted LNP-mRNA

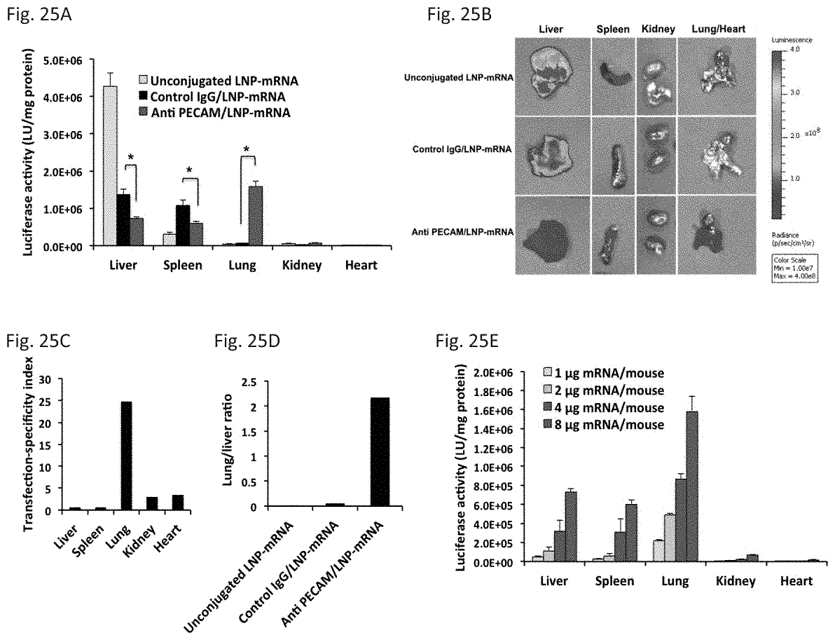

Protein expression with mRNA delivery is expected to peak around 4-5 h after i.v. injection (Pardi et al., 2015, J. Control. Release., 217:345-351). Therefore, 4.5 h following injection, bioluminescence and luciferase assay were used to investigate the biodistribution and levels of protein expression from the delivered mRNA. Retro-orbital injection of 8 μg (0.32 mg/kg) of unconjugated LNP-mRNA led to firefly luciferase expression mainly in liver and at lower level in spleen (FIG. 25A and FIG. 25B). PECAM-targeted Ab/LNP-mRNA, but not IgG/LNP-mRNA showed profound and specific protein expression in the lung concomitant with reduced hepatic expression, providing an ˜25 fold elevation of the reporter protein signal in the lungs compared to untargeted counterparts (FIG. 25A and FIG. 25C). The lung/liver ratio for Ab/LNP-mRNA increased ˜200 and 50-fold compared to unconjugated LNP-mRNA and IgG/LNP-mRNA, respectively (FIG. 25D). Dose escalation studies demonstrated that within the range of injected doses, expression of the signal does not reach saturation (FIG. 25E). This aligns well with the known high level of surface expression of PECAM in endothelial cells (Scherpereel et al., 2002, J. Pharmacol. Exp. Ther., 300:777-786; Ding et al., 2009, Am. J. Respir. Crit. Care Med., 180:247-256; Scherpereel et al., 2001, FASEB J., 15(2):416-426).

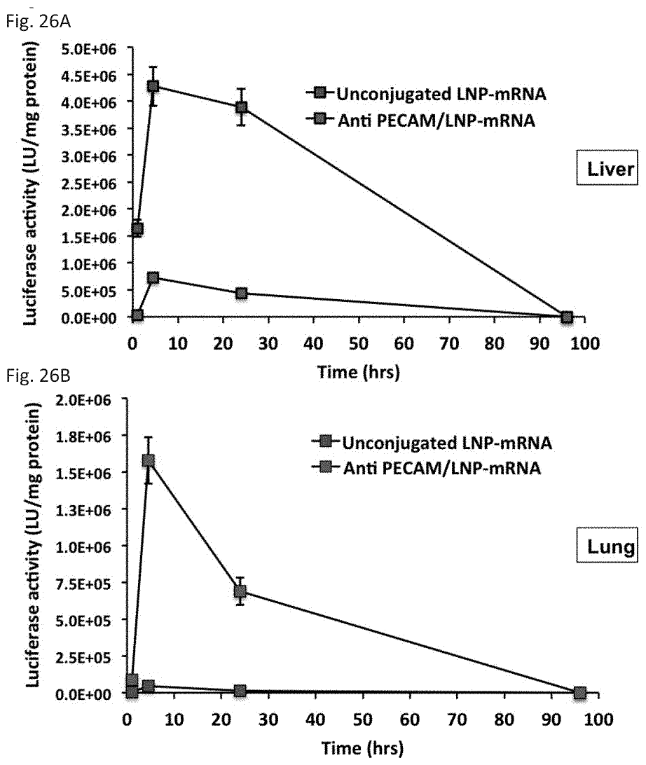

The time course of mRNA translation was further characterized using luciferase mRNA containing LNPs. Four time points, 1, 4.5, 24, and 96 hours post-injection were chosen, based on previous studies (Pardi et al., 2015, J. Control. Release., 217:345-351). It is notable that all three formulations, unconjugated-, IgG-, and anti-PECAM/LNP-mRNAs reached their maximal expression after 4.5 hours and declined slowly in the next 24 hours (FIG. 26).

Ab/LNP-mRNA Targeting is Independent of the apoE Pathway(s) Involved in Hepatic Delivery of Untargeted LNP-mRNA

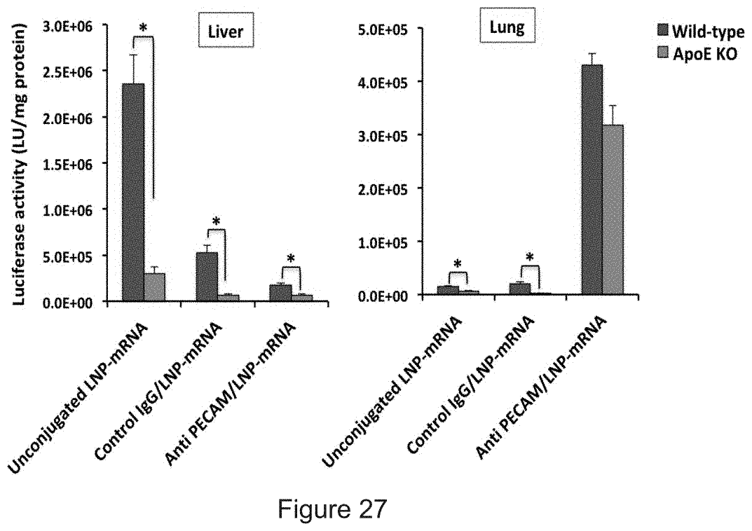

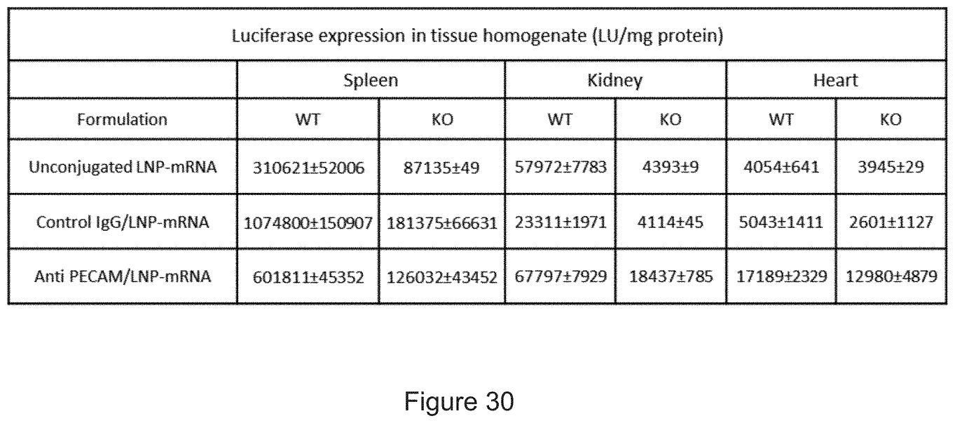

Interaction with various compounds in blood modulates the behavior of nanocarriers (Semple et al., 1998, Adv. Drug Deliv. Rev., 32:3-17). For example, apolipoprotein E (apoE) mediates hepatic uptake of LNP-mRNA (Akinc et al, 2010, Mol. Ther., 18:1357-1364). Here, it is demonstrated in apoE^{-/-} mice that affinity moieties like anti-PECAM could maintain targeting to desired tissues, lung, in the absence of apoE. Indeed, the hepatic luminescence signal measured after firefly luciferase mRNA-LNP delivery, was an order of magnitude lower in Apo-E^{-/-} mice compared to wild type animals (FIG. 27). Similarly, lower signal intensities were observed in other organs (FIG. 30). While not wishing to be bound by any particular theory, one potential explanation for the observation that apo E KO inhibits hepatic uptake without redistribution to other organs is that LNPs apparently have no or little affinity to other organs. For this reason, clearance mechanisms outcompete inefficient distribution of LNPs from blood to the organs in apo E KO mice. In sharp contrast, anti-PECAM/LNPs that have affinity to endothelium, do accumulate in the lungs of KO mice. No significantly reduced firefly luciferase activity in the lungs of apo-E^{-/-} mice was observed after PECAM-1 targeted Ab/LNP-mRNA delivery compared to wild-type animals (FIG. 27). This further highlights the significance of targeting moieties in mitigating the influence of endogenous serum components like apoE from averting the transfection from desired tissue to liver.

### CONCLUSION

Systemic mRNA delivery using antibody-coupled nucleoside-modified mRNA-LNPs provides highly effective vascular immunotargeting to target organs other than the liver in mice. The presently described targeted delivery platform was designed with one of the most efficient LNP-mRNA systems developed, thus far, that uses modified nucleosides to decrease innate immune activation and increase protein translation from mRNA in vivo (Karikó et al., 2008, Mol. Ther., 16:1833-1840; Pardi et al., 2017, Nat. Commun., 8:14630; Karikó et al., 2005, Immunity., 23:165-175). Specific, rapid, and transient protein expression from firefly luciferase-encoding mRNA was measurable in the lungs at a time window of 4-24 hours, with limited off-target biodistribution. Flow cytometry analysis showed efficient uptake in endothelial cells. Notably, we demonstrated that antibody-targeted LNC-mRNAs is independent of the apo-E mediated uptake pathway(s). These studies provide the basis for development of targeted delivery systems for mRNA therapeutics in pulmonary conditions, including acute lung injury, pulmonary hypertension, and beyond. Taking into consideration the diversity and extension of endothelial phenotypes in the vascular system, and the pivotal roles played by these cells under physiologic and pathologic conditions, targeting strategies for vascular delivery of mRNA will find widespread medical utility.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a delivery vehicle conjugated to a targeting domain, wherein the delivery vehicle comprises at least one agent, and wherein the targeting domain specifically binds to an endothelial marker of the vasculature, wherein the marker is selected from the group consisting of ICAM-1, PECAM-1, VCAM-1, ACE, APP, PV1, P-selectin, E-selectin, and VE-cadherin.

2. The composition of claim 1, wherein the delivery vehicle is selected from the group consisting of a liposome, a lipid nanoparticle, and a micelle.

3. The composition of claim 1, wherein the delivery vehicle is a lipid nanoparticle.

4. The composition of claim 3, wherein the lipid nanoparticle comprises a PEG-lipid conjugated to the targeting domain.

5. The composition of claim 3, wherein the at least one agent is encapsulated in the lipid nanoparticle.

6. The composition of claim 1, wherein the at least one agent is selected from the group consisting of a therapeutic agent, an imaging agent, diagnostic agent, a contrast agent, a labeling agent, a detection agent, and a disinfectant.

7. The composition of claim 1, wherein the at least one agent is a therapeutic agent.

8. The composition of claim 7, wherein the therapeutic agent comprises a nucleic acid molecule.

9. The composition of claim 8, wherein the nucleic acid molecule encodes a therapeutic peptide selected from the group consisting of a thrombomodulin, endothelial protein C receptor, an anti-thrombotic protein, a plasminogen activator, catalase, superoxide dismutase, and an iron-sequestering protein.

10. The composition of claim 1, wherein the targeting domain is selected from the group consisting of a nucleic acid molecule, a peptide, an antibody, and a small molecule.

11. The composition of claim 1, wherein the targeting domain is an antibody.

12. The composition of claim 1, wherein the targeting domain specifically binds to platelet-endothelial cell adhesion molecule-1 (PECAM-1).

13. The composition of claim 1, wherein the targeting domain specifically binds to vascular cell adhesion molecule-1 (VCAM-1).

14. The composition of claim 1, wherein the targeting domain specifically binds to intercellular adhesion molecule-1 (ICAM-1).

15. A method of treating or preventing a neurological condition of a subject, the method comprising administering to the subject the composition of claim 13.

16. The method of claim 15, wherein the neurological condition is selected from the group consisting of stroke, inflammation, infection, meningitis, traumatic brain injury, multiple sclerosis, concussion, cerebral embolism, hemorrhage, brain tumors, neurodegenerative disorders, depression, post-traumatic stress disorder, anxiety, mood disorders, and addiction disorders.

17. A method of treating or preventing a pulmonary condition of a subject, the method comprising administering to the subject a composition selected from the group consisting of: (a) a composition comprising a delivery vehicle conjugated to a targeting domain, wherein the delivery vehicle comprises at least one agent, and wherein the targeting domain specifically binds to PECAM-1; and (b) a composition comprising a delivery vehicle conjugated to a targeting domain, wherein the delivery vehicle comprises at least one agent, and wherein the targeting domain specifically binds to ICAM-1.

18. The method of claim 17, wherein the pulmonary condition is selected from the group consisting of acute lung injury, pulmonary ischemia including organ transplantation, pulmonary embolism, pulmonary edema, pulmonary hypertension, fibrosis, infection, inflammation, emphysema, and cancer.
